(19) United States
(12) Patent Application Publication
(10) Pub. No.: US 2003/0149005 A1
(43) Pub. Date: Aug. 7, 2003

(54) 24-sulfur-substituted analogs of 1alpha, 25-dihydroxy vitamin D3

(76) Inventors: Gary H. Posner, Baltimore, MD (US); Kenneth R. Crawford, Decatur, GA (US); Hong Woon Yang, Baltimore, MD (US); Jay A. White, Newmarket (CA); Glenville Jones, Kingston (CA); Byung-Chul Suh, Cockeysville, MD (US); HeungBae Jeon, Baltimore, MD (US); Mark Hatcher, Baltimore, MD (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/225,475

(22) Filed: Aug. 22, 2002

Related U.S. Application Data
(60) Provisional application No. 60/313,769, filed on Aug. 22, 2001.
(60) Provisional application No. 60/328,429, filed on Oct. 12, 2001.

(51) Int. Cl. A61K 31/59; C07C 401/00; C07D 213/26

(52) U.S. Cl. 514/167; 552/653; 546/329

(57) ABSTRACT

The present invention provides novel C24-aryl sulfone analogs of 1α,25-dihydroxy vitamin D₃, compositions comprising these compounds and methods of using these compounds as selective inhibitors of CYP24. In particular, the compounds of the invention are useful for treating diseases which benefit from a modulation of the levels of 1α,25-dihydroxy vitamin D₃, for example, cell-proliferative disorders.

### CROSS REFERENCE TO RELATED APPLICATIONS

This application claims the benefit under 35 USC §119(e) from U.S. provisional patent application S. No. 60/313,769, filed Aug. 22, 2001; U.S. provisional patent application S. No. 60/328,429, filed Oct. 12, 2001; and U.S. provisional patent application entitled “24-Aryl Sulfone Analogs of 1α,25-Dihydroxy Vitamin D₃” filed Jun. 13, 2002, the contents of which are incorporated herein by reference in their entirety.

This invention was made with government support under NIH Grant Number CA 44530. The government has certain rights in the invention.

### FIELD OF THE INVENTION

The present invention relates to novel analogs of the hormone 1α,25-dihydroxy vitamin D₃ that show selective inhibition of the enzyme CYP24 and which are low-calcemic, to pharmaceutical and diagnostic compositions containing them and to their medical use, particularly in the treatment and/or prevention of cancer, dermatological disorders, bone disorders, thyroid disorders, wound healing and osteoporosis.

### BACKGROUND OF THE INVENTION

The vitamin D metabolic pathway is part of a vital endocrine system that is highly regulated at certain stages and produces metabolites that control the secretion of the parathyroid gland hormones (Beckman, M., and DeLuca, H. (1997) Methods in Enzymol. 282, 200-223; Jones, G., Strugnell, S., and DeLuca, H. (1998) Physiol. Rev. 78, 1193-1231). 1α,25-Dihydroxy vitamin D₃, also known as calcitriol (see below), a hormone produced in the vitamin D pathway, regulates phosphate and calcium levels in the blood which in turn control bone mass, the state of bones, and affects cellular differentiation in the skin and the immune system (Armbrecht, H. J., Okuda, K., Wongsurawat, N., Nemani, R., Chen, M., and Boltz, M. (1992) J. Steroid Biochem. Molec. Biol. 43, 1073-1081). In the vitamin D pathway, cytochrome P450s are enzymes that introduce functional groups by hydroxylation, usually at positions 1, 25, and 24, of vitamin D₃ (Beckman, M., and DeLuca, H. (1997) Methods in Enzymol. 282, 200-223).

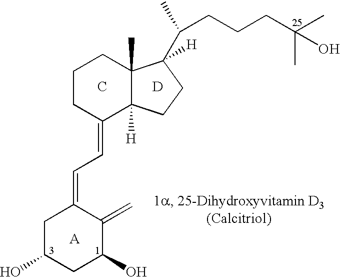

1α,25-Dihydroxy vitamin D₃ is converted to 1α,24,25-trihydroxy-D₃ by a mitochondrial P450 known as CYP 24 (Bell, N. H., (1998) J. Bone Miner. Res. 13, 350-35211). CYP 24 is induced by 1α,25-dihydroxy-D₃ and is found in the kidney as well as other vitamin D target tissues such as the parathyroid cells, keratinocytes, osteoblasts, and enteroctyes (Jones, G., Strugnell, S., and DeLuca, H. (1998) Physiol. Rev. 78, 1193-1231). 1α,25-Dihydroxy vitamin D₃ (1α,25-D3) has an important role in the antiproliferative and growth regulatory effects on normal and neoplastic cells (for e.g. prostate cancer cells). Clinical use of 1α,25-D3 analogs as effective drugs requires separating desirable antiproliferative and pro-differentiating activities from undesirable calcemic activity. There is a continuing need for synthetic analogs of 1α,25-dihydroxy vitamin D₃ that selectively exhibit desirable pharmacological activities but do not exhibit hypercalcemic and other undesirable activity.

### SUMMARY OF THE INVENTION

It has been found that 24-aryl sulfone analogs of 1α,25-dihydroxy vitamin D₃ show selective inhibition of the enzyme CYP24.

The present invention therefore provides compounds of Formula I, and pharmaceutically acceptable salts, hydrates, solvates and prodrugs thereof:

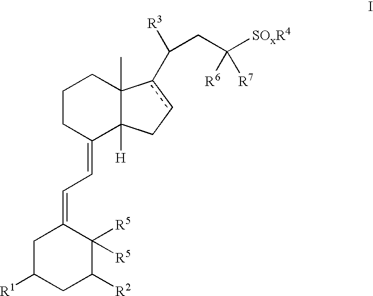

wherein

R¹ and R² are independently selected from the group consisting of OH, OC₁₋₄alkyl, and halo;

R³ is C₁₋₄alkyl;

R⁴ is selected from the group consisting of C₁₋₆alkyl, aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, CH═N—OC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;

R⁵ are either both H or together form ═CH₂;

R⁶ and R⁷ are independently H, C₁₋₄alkyl or are taken together to form a C₃₋₆cyloalkyl ring;

x is 0-2; and

[symbol] represents a single or a double bond.

In an embodiment, the present invention provides compounds of Formula I wherein the stereochemistry is that of natural 1α,25-dihydroxy vitamin D₃. Accordingly, the present invention relates to a compound of Formula I, and pharmaceutically acceptable salts, hydrates, solvates and prodrugs thereof:

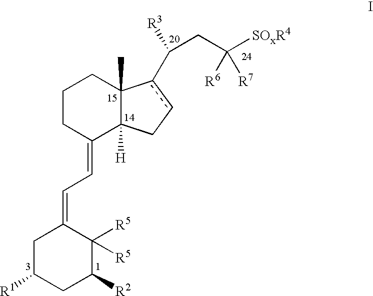

wherein

R¹ and R² are independently selected from the group consisting of OH, OC₁₋₄alkyl, and halo;

R³ is C₁₋₄alkyl;

R⁴ is selected from the group consisting of C₁₋₆alkyl, aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, CH═N—OC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;

R⁵ are either both H or together form ═CH₂;

R⁶ and R⁷ are independently H, C₁₋₄alkyl or are taken together to form a C₃₋₆cyloalkyl ring;

x is 0-2; and

[symbol] represents a single or a double bond.

In a further embodiment of the invention, the compounds of Formula I are those wherein R⁶ and R⁷ are H. Accordingly, the present invention relates to a compound of Formula I, and pharmaceutically acceptable salts, hydrates, solvates and prodrugs thereof:

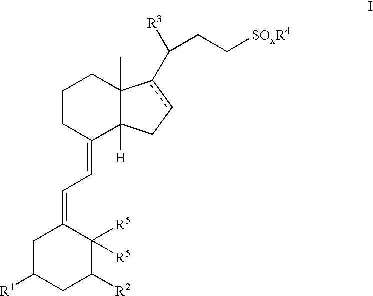

wherein

R¹ and R² are independently selected from the group consisting of OH, OC₁₋₄alkyl, and halo;

R³ is C₁₋₄alkyl;

R⁴ is selected from the group consisting of C₁₋₆alkyl, aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, CH═N—OC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;

R⁵ are either both H or together form ═CH₂;

x is 0-2; and

[symbol] represents a single or a double bond.

According to another aspect of the present invention, there is provided a pharmaceutical composition comprising a compound of the invention and a pharmaceutically acceptable carrier or diluent.

By selectively modulating CYP24, the enzyme that metabolizes 1α,25-dihydroxy vitamin D₃, the levels of 1α,25-dihydroxy vitamin D₃ may also be modulated. Diseases that benefit from a modulation of the levels of 1α,25-dihydroxy vitamin D₃ can therefore be treated using a modulator of CYP24. By acting preferentially on CYP24, side effects caused by interaction with other enzymes and receptors may be reduced. Accordingly, the present invention provides a method for treating diseases which benefit from a modulation of the levels of 1α,25-dihydroxy vitamin D₃ comprising administering an effective amount of a compound of the invention to a cell or animal in need thereof. The invention also includes the use of a compound of the invention to modulate the levels of 1α,25-dihydroxy vitamin D₃. Further, the invention includes a use of a compound of the invention to prepare a medicament to modulate the levels of 1α,25-dihydroxy vitamin D₃.

Inhibition of CYP24, should inhibit the catabolism of 1α,25-dihydroxy vitamin D₃ which is expected to lengthen the biological lifetime of this hormone and thus allow smaller amounts of it to be used for effective disease treatment. Such smaller dosing is expected to avoid, or at least minimize, the hypercalcemic toxicity associated with medicinal use of 1α,25-dihydroxy vitamin D₃ (calcitriol). Therefore, in a preferred embodiment, the present invention provides a method for treating diseases which benefit from inhibiting the catabolism of 1α,25-dihydroxy vitamin D₃ comprising administering an effective amount of a compound of the invention to a cell or animal in need thereof. The invention also includes the use of a compound of the invention to inhibit the catabolism of 1α,25-dihydroxy vitamin D₃. Further, the invention includes a use of a compound of the invention to prepare a medicament to inhibit the catabolism of 1α,25-dihydroxy vitamin D₃.

Diseases which may benefit for a modulation in the levels of 1α,25-dihydroxy vitamin D₃ include, but are not limited to:

(i) in the parathyroid—hyper- and hypo-parathyroidism, Osudohypoparathyroidism, Secondary hyperparathyroidism;

(ii) in the pancreas—diabetes;

(iii) in the thyroid—medullary carcinoma;

(iv) in the skin—psoriasis, wound healing;

(v) in the lung—sarcoidosis and tuberculosis;

(vi) in the kidney—chronic renal disease, hypophosphtatemic VDRR, vitamin D dependent rickets;

(vii) in the bone—anticonvulsant treatment, fibrogenisis imperfecta ossium, osteitits fibrosa cystica, osteomalacia, osteporosis, osteopenia, osteosclerosis, renal osteodytrophy, rickets;

(viii) in the intestine—glucocorticoid antagonism, idopathic hypercalcemia, malabsorption syndrome, steatorrhea, tropical sprue.

In accordance with a further aspect of the present invention, there is provided a method for modulating cell proliferation, preferably inhibiting cell proliferation comprising administering an effective amount of a compound of the invention to a cell or animal in need thereof. The invention also includes a use of a compound the invention to modulate cell proliferation, preferably inhibit cell proliferation. The invention further includes a use of a compound of the invention to prepare a medicament to modulate cell proliferation, preferably inhibit cell proliferation.

In a preferred embodiment, the present invention provides a method of inhibiting the proliferation of a cancer cell comprising administering an effective amount of a compound of the invention to a cell or animal in need thereof. The invention also includes a use of a compound of the invention to modulate cancer cell proliferation, preferably inhibit cancer cell proliferation. The invention further includes a use of a compound of the invention to prepare a medicament to modulate cancer cell proliferation, preferably inhibit cancer cell proliferation.

In another aspect, the invention provides a method of modulating CYP24 activity in a cell by administering an effective amount of a compound of the invention. In a further aspect, the invention provides a method of inhibiting CYP24 activity in a cell by administering an effective amount of a compound of the invention. The present invention also provides a use of a compound of the invention to modulate, preferably inhibit, CYP24 activity. The present invention further provides a use of a compound of the invention to prepare a medicament to modulate CYP24 activity, preferably inhibit CYP24 activity. It is appreciated that the inhibition of cell growth by the compounds of the invention may be effected by other mechanisms.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1A is a graph showing the inhibition of CYP24 activity by compound I(a) (indicated as KRC24SO₂Ph-1) compared to ketoconazole.

FIG. 1B is a graph showing the inhibition of CYP27B 1 activity by compound I(a) (indicated as KRC24SO₂Ph-1) compared to ketoconazole.

FIG. 1C is a graph showing the inhibition of CYP27A1 activity by compound I(a) (indicated as KRC24SO₂Ph-1) compared to ketoconazole.

FIG. 2 is a graph showing the binding of compound I(a) (indicated as KRC24SO₂Ph-1) compared to 1α,25-dihydroxy vitamin D₃ at the vitamin D receptor.

FIG. 3 is a graph showing the activity of compound I(a) (indicated as KRC24SO₂Ph-1) in the vitamin D transcription assay compared to 1α,25-dihydroxy vitamin D₃.

FIG. 4 is a graph showing the activity of compound I(a) (indicated as KRC24SO₂Ph-1) in the DBP binding assay compared to 1α,25-dihydroxy vitamin D₃.

### DETAILED DESCRIPTION OF THE INVENTION

I. Definitions

The term “C₁₋₄alkyl” as used herein means straight and/or branched chain alkyl groups containing from one to four carbon atoms and includes methyl, ethyl, propyl, isopropyl, t-butyl and the like.

The term “hydroxy-substituted C₁₋₄alkyl” as used herein means straight and/or branched chain alkyl groups containing from one to four carbon atoms and substituted with 1-2 hydroxyl groups and includes hydroxymethyl, 1-hydroxyethyl, 2-hydroxyl-2-propyl and the like.

The term “C₁₋₄alkoxy” as used herein means straight and/or branched chain alkoxy groups containing from one to four carbon atoms and includes methoxy, ethoxy, propyoxyl, isopropyloxy, t-butoxy and the like.

The term “C₃₋₆cycloalkyl” as used herein means a 3- to 6-membered saturated carbocyclic ring.

The term “aryl” as used herein means unsubstituted or substituted mono- or bicyclic aromatic groups containing from 6 to 10 carbon atoms and includes phenyl and naphthyl and the like.

The term “heteroaryl” as used herein means unsubstituted or substituted mono- or bicyclic heteroaromatic groups containing from 5 to 10 atoms, of which 1-3 atoms may be a heteroatom selected from the group consisting of S, O and N, and includes furanyl, thienyl, pyrrolo, pyridyl, indolo, benzofuranyl and the like.

The term “halo” as used herein means halogen and includes chloro, flouro, bromo, iodo and the like.

The term “pharmaceutically acceptable salt” means an acid addition salt or a basic addition salt which is suitable for or compatible with the treatment of patients.

The term “pharmaceutically acceptable acid addition salt” as used herein means any non-toxic organic or inorganic salt of any base compound of the invention, or any of its intermediates. Basic compounds of the invention that may form an acid addition salt include those where R⁴ is substituted with a group having a basic nitrogen, for example NH₂ and NHC₁₋₄alkyl. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compounds of the invention are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the invention, for laboratory use, or for subsequent conversion to a pharmaceutically acceptable acid addition salt.

The term “pharmaceutically acceptable basic addition salt” as used herein means any non-toxic organic or inorganic base addition salt of any acid compound of the invention, or any of its intermediates. Acidic compounds of the invention that may form a basic addition salt include those where R⁴ is substituted with a group having acidic hydrogen, for example C(O)OH. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art.

The term “solvate” as used herein means a compound of the invention, or a pharmaceutically acceptable salt of a compound of the invention, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The term “compound(s) of the invention” as used herein means compound(s) of Formula I, and salts, hydrates, solvates and prodrugs thereof.

The term an “effective amount” or a “sufficient amount” of an agent as used herein is that amount sufficient to effect beneficial or desired results, including clinical results, and, as such, an “effective amount” depends upon the context in which it is being applied. For example, in the context of administering an agent that inhibits cancer cell proliferation, an effective amount of an agent is, for example, an amount sufficient to achieve such a reduction in cancer cell proliferation as compared to the response obtained without administration of the agent.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

“Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

The term “modulate” as used herein includes the inhibition or suppression of a function or activity (such as cell proliferation) as well as the enhancement of a function or activity.

To “inhibit” or “suppress” or “reduce” a function or activity, such as cancer cell proliferation, is to reduce the function or activity when compared to otherwise same conditions except for a condition or parameter of interest, or alternatively, as compared to another conditions.

The term “animal” as used herein includes all members of the animal kingdom including human. The animal is preferably a human.

The term “a cell” as used herein includes a plurality of cells. Administering a compound to a cell includes in vivo, ex vivo and in vitro treatment.

The term “cancer cells” as used herein includes all forms of cancer or neoplastic disease.

II. Compounds of the Invention

Novel compounds showing selective inhibition of the enzyme CYP24 have been prepared. As such, the compounds of the invention are useful for treating cell proliferative diseases, such as cancer.

Accordingly, the present invention provides compounds of Formula I, and pharmaceutically acceptable salts, hydrates, solvates and prodrugs thereof:

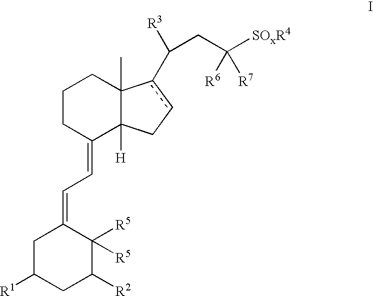

wherein

R¹ and R² are independently selected from the group consisting of OH, OC₁₋₄alkyl, and halo;

R³ is C₁₋₄alkyl;

R⁴ is selected from the group consisting of C₁₋₆alkyl, aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, CH═N—OC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;

R⁵ are either both H or together form ═CH₂;

R⁶ and R⁷ are independently H, C₁₋₄alkyl or are taken together to form a C₃₋₆cyloalkyl ring;

x is 0-2; and

[symbol] represents a single or a double bond.

The compounds of Formula I include those in which R¹ and R² are independently selected from the group consisting of OH, OC₁₋₄alkyl, and halo. In embodiments of the invention, R¹ and R² are independently selected from the group consisting of OH, OCH₃, and fluoro. In a further embodiment, R¹ and R² are both OH.

The present invention includes compounds of Formula I wherein R³ is C₁₋₄alkyl. In embodiments of the invention, R³ is CH₃.

The present invention includes compounds of Formula I wherein R⁴ is selected from the group consisting of C₁₋₆alkyl, aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, CH═N—OC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂. In embodiments of the invention, R⁴ is selected from C₁₋₆alkyl, unsubstituted or substituted phenyl, pyridyl, thienyl, furanyl and pyrrolo. In further embodiments, R⁴ is selected from C₁₋₄alkyl, unsubstituted or substituted phenyl. In still further embodiments of the present invention, both aryl and heteroaryl may be either unsubstituted or substituted with 1-3 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, CH═N—OC₁₋₄alkyl, C(O)NHC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂. Preferably the substituent is located at a position other than that ortho to the SO₂ group. In further embodiments, both aryl and heteroaryl may be either unsubstituted or substituted with 1-2 groups independently selected from methyl, 3-hydroxy-3-pentyl, methoxy, OH, CF₃, OCF₃, halo, NH₂, NMe₂ and CH═N—OMe. In further embodiments, both aryl and heteroaryl may be either unsubstituted or substituted with 1-2 groups independently selected from methyl, 3-hydroxy-3-pentyl, Cl, F and CH═N—OMe. In specific embodiments of the invention, R⁴ is selected from the group consisting of methyl, ethyl, n-propyl, t-butyl, isopropyl, isobutyl, phenyl, 4-chlorophenyl, 3,4-dichloropheny, 4-fluorophenyl, 4-methylphenyl, 3,4-difluorophenyl, 4-(3-hydroxy-3-pentyl)phenyl, 4-(CH═N—OMe)phenyl, 4-methoxyphenyl, 4-trifluormethylpheny and 4-ntirophenyl. In more specific embodiments of the invention, R⁴ is selected from the group consisting of t-butyl, isopropyl, phenyl, 4-chlorophenyl, 3,4-dichloropheny, 4-(3-hydroxy-3-pentyl)phenyl, 4-fluorophenyl and 4-methylphenyl.

The compounds of Formula I include those where R⁵ are either both H or, together, R⁵ form the group ═CH₂.

The compounds of Formula I include those where R⁶ and R⁷ are independently H, C₁₋₄alkyl or are taken together to form a C₃₋₆cyloalkyl ring. In embodiments of the invention, R⁶ and R⁷ are independently H, methyl or are taken together to form a C₃₋₄cyloalkyl ring. In further embodiments of the invention, R⁶ and R⁷ are both H or are taken together to form a C₃₋₄cyloalkyl ring.

The present invention further includes compounds of Formula I wherein x is 0-2. In embodiments of the invention, x is 2.

The present invention also includes compounds of Formula I wherein [symbol] represents a single or a double bond. When [symbol] represents a double bond, it is an embodiment of the invention that R⁴ is C₁₋₆alkyl.

All of the compounds of Formula I have more than one asymmetric centre. Where the compounds according to the invention possess more than one asymmetric centre, they may exist as diastereomers. It is to be understood that all such isomers and mixtures thereof in any proportion are encompassed within the scope of the present invention. The stereochemistry of the compounds of the invention is preferably that of natural 1α,25-dihydroxy vitamin D₃. Therefore, in an embodiment, the present invention provides compounds of Formula I, and pharmaceutically acceptable salts, hydrates, solvates and prodrugs thereof:

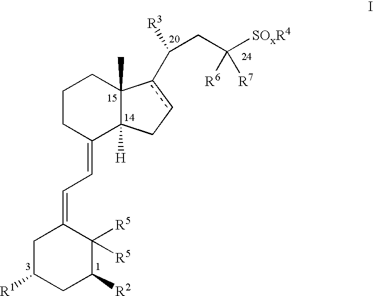

wherein

R¹ and R² are independently selected from the group consisting of OH, OC₁₋₄alkyl, and halo;

R³ is C₁₋₄alkyl;

R⁴ is selected from the group consisting of aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl; OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;

R⁵ are either both H or together form ═CH₂;

R⁶ and R⁷ are independently H. C₁₋₄alkyl or are taken together to form a C₃₋₆cyloalkyl ring;

x is 0-2; and

[symbol] represents a single or a double bond.

When [symbol] is a single bond in the compounds of Formula I, it is an embodiment of the invention that the stereochemistry at carbon 17 is that of natural 1α,25-dihydroxy vitamin D₃ (i.e. R). It is to be understood that, while the relative stereochemistry for compounds of Formula I in an embodiment of the invention, is that of natural 1α,25-dihydroxy vitamin D₃, such compounds may contain certain amounts of the unnatural isomer, for example, less than about 25%, preferably less than about 20%, more preferably, less than about 10%.

In a further embodiment of the invention, the compounds of Formula I are those wherein R⁶ and R⁷ are H. Accordingly, the present invention relates to a compound of Formula I, and pharmaceutically acceptable salts, hydrates, solvates and prodrugs thereof:

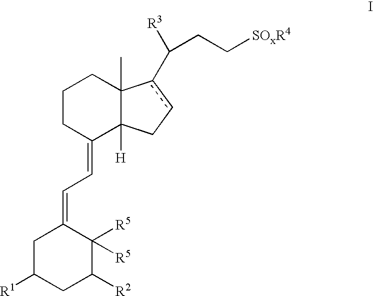

wherein

R¹ and R² are independently selected from the group consisting of OH, OC₁₋₄alkyl, and halo;

R³ is C₁₋₄alkyl;

R⁴ is selected from the group consisting of C₁₋₆alkyl, aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, CH═N—OC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;

R⁵ are either both H or together form ═CH₂;

x is 0-2; and

[symbol] represents a single or a double bond.

In specific embodiments of the present invention, the compounds of the invention include:

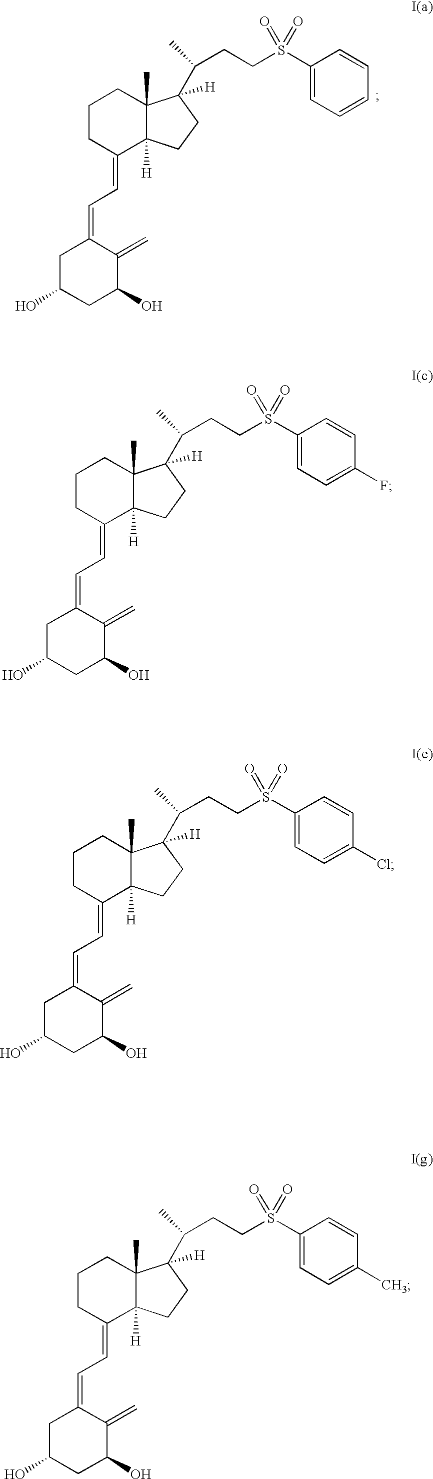

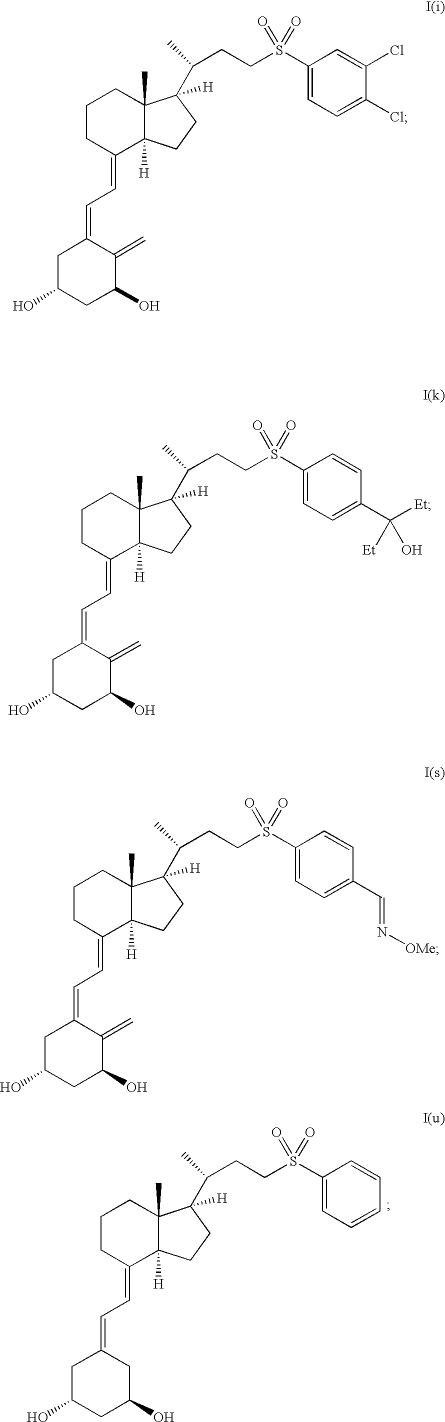

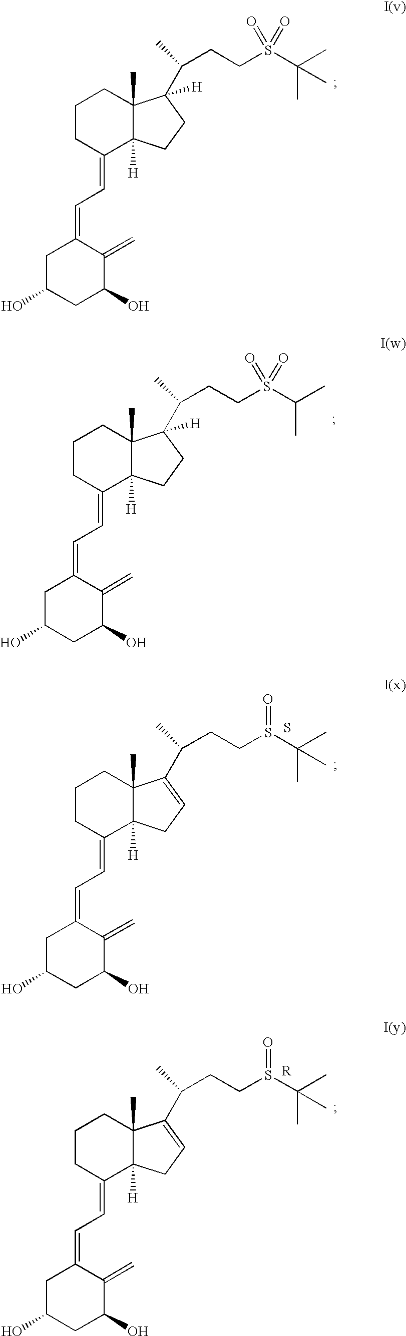

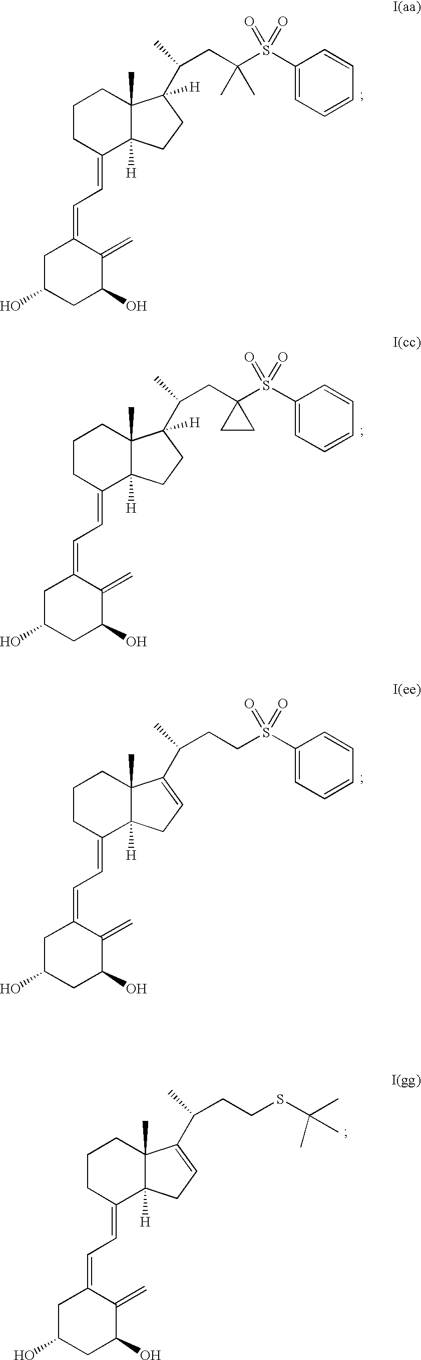

and pharmaceutically acceptable salts, hydrates, solvates and prodrugs thereof.

III. Methods of Preparing Compounds of the Invention

In accordance with another aspect of the present invention, the compounds of the invention can be prepared by processes analogous to those established in the art. Therefore, compounds of this invention may be prepared, for example, by the reaction sequence shown in Scheme 1:

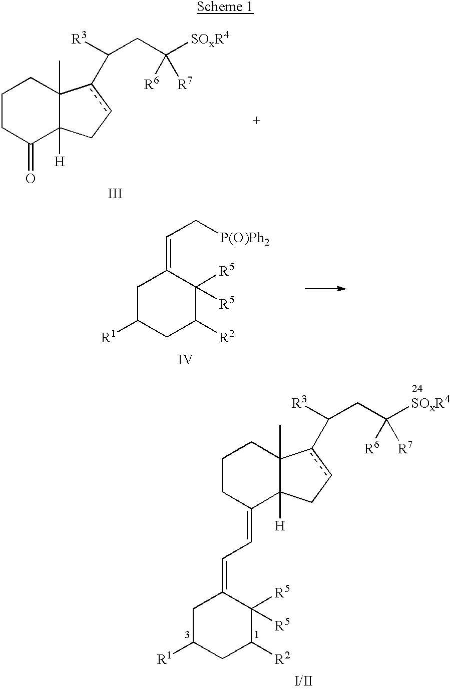

Ketones of Formula III, wherein R³, R⁴, R⁵, R⁶, x and [symbol] are as defined in Formulae I ad II, may be reacted with phosphine oxides of Formula IV, wherein R¹, R² and R⁵ are as defined in Formula I, under standard Horner-Wadsworth-Emmons (HWE) coupling conditions. Therefore phosphine oxides IV, wherein R¹, R² and R⁵ are as defined in Formula I, are treated with a strong base, for example an alkyl lithium such as n-butyl lithum, under anhydrous conditions in an inert atmosphere and solvent, for example tetrahydrofuran (THF), at temperatures in the range of about 60° C. to about −90° C., suitably at about −78° C. To the resulting intermediate ylide is added a cold, preferably at about −78° C., solution of a ketone III in an inert solvent such as THF while maintaining the anhydrous conditions. After removal of any protecting groups using standard chemistries (if needed), compounds of Formula I may be obtained.

Ketones of Formula III, wherein wherein R³, R⁴, R⁵, R⁶, x and [symbol] are as defined in Formula I, may be prepared, for example, as shown in Scheme 2:

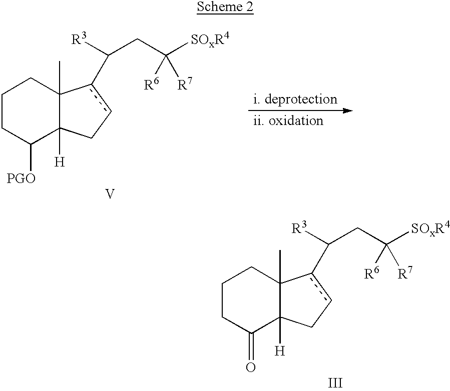

Suitably protected oxysulfones V, wherein R³, R⁴, R⁵, R⁶, x and [symbol] are as defined in Formula I and PG is a suitable protecting group, are first deprotected and then oxidized to provide ketones III, wherein R³, R⁴, R⁵, R⁶, x and [symbol] are as defined in Formula I. For example, when PG is trialkyl silyl, such as triethyl silyl, deprotection may be affected by reacting compounds of Formula V with tetrabutylammonium fluoride (TBAF) in an inert solvent, such as THF, and in an inert atmosphere, suitably at about room temperature. Oxidation of the resulting alcohol may be performed, for example, using pyridinium dichromate (PDC), tetrapropylammonium perruthenate (TPAP)/morpholine N-oxide (NMO), or any other suitable oxidizing agent, in an inert solvent such as methylene chloride, under standard conditions.

Compounds of Formula V, wherein R³, R⁴, R⁶, R⁷, x and [symbol] are as defined in Formula I and PG is a suitable protecting group, may be obtained, for example, as shown in Scheme 3:

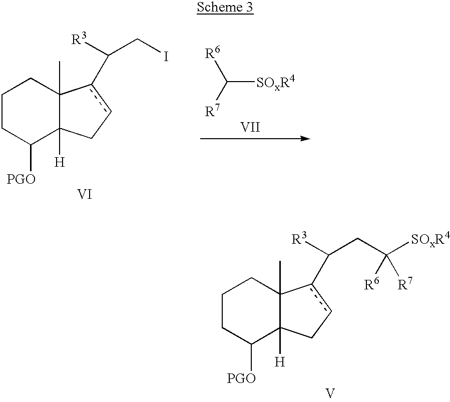

Compounds of Formula VI, wherein R³ and [symbol] are as defined in Formula I and PG is a suitable protecting group, may be reacted with the anion of compounds of Formula VII, wherein R⁴, R⁶, R⁷, x and [symbol] are as defined in Formula I, under anhydrous conditions at temperatures in the range of about −60° C. to about −90° C., suitably at about −78° C. The anions of compounds of Formula VII may be prepared by treating compounds of Formula VII with a strong base, for example an alkyl lithium such as n-butyl lithium, under inert conditions and, in the presence of hexamethyl phosphoramide (HMPA), for example, or N,N,N¹,N¹-tetramethylethylenediamine (TMEDA).

Compounds of Formula VII, wherein R⁴, R⁶ and R⁷ are as defined in Formula I and x is 1 or 2, are either commercially available or may be prepared, for example, by the oxidation of the corresponding compounds of Formula VII, wherein R⁴, R⁶ and R⁷ are as defined in Formula I and x is 0, as shown in Scheme 4. Suitable oxidizing agents include Ozone®, m-chloroperbenzoic acid and RuCl₃.H₂O/periodic acid (H₅IO₆). The use of sterically hindered oxidizing reagents assists in the isolation of the sulfoxide (i.e. compounds of Formula VII, where x=1). An example of such an oxidizing reagent is camphorsulfonyl oxaziridine (available as pure enantiomers which can lead to the formation of enantiomerically enriched sulfoxides).

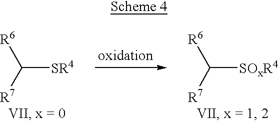

Compounds of Formula VII, wherein R⁴, R⁶ and R⁷ are as defined in Formula I and x is 0, are either commercially available or may be prepared, for example, as shown in Scheme 5. Therefore a reagent of Formula VIII, wherein R⁶ and R⁷ are as defined in Formula I and LG is a suitable leaving group, such as halogen, may be reacted with a compound of Formula IX, wherein R⁴ is as defined in Formula I, in the presence of a base, for example sodium methoxide and an inert solvent, to provide compounds of Formula VII, wherein R⁴, R⁶ and R⁷ are as defined in Formula I and x is 0.

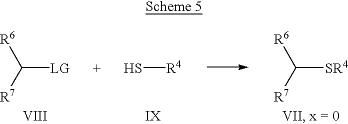

An alternate route to the compounds of Formula V, wherein R³, R⁴, x and [symbol] are as defined in Formula I, R⁶ and R⁷ are H and PG is a suitable protecting group, is shown in Scheme 6. Accordingly, a compound of Formula X wherein R³ and [symbol] are as defined in Formula I and PG is a suitable protecting group, may be reacted with a compound of Formula IX, wherein R⁴ is as defined in Formula I, in the presence of a base such as 1,8-diazabicyclo[5.4.0]undec-7-ene (DBU), at elevated temperatures, such as about 110-150° C., suitably at about 130° C., in an inert, high-boiling solvent, such as benzene, to provide a compound of Formula V, wherein R³, R⁴, and [symbol] are as defined in Formula I, R⁶ and R⁷ are H, x is 0 and PG is a suitable protecting group. Oxidation of a compound of Formula V, wherein R³, R⁴, and [symbol] are as defined in Formula I, R⁶ and R⁷ are H, x is 0 and PG is a suitable protecting group, with suitable oxidizing agents, provides compounds of Formula V, wherein R³, R⁴, and [symbol] are as defined in Formula I, R⁶ and R⁷ are H, x is 1 or 2 and PG is a suitable protecting group. Suitable oxidizing agents include, for example Ozone®, m-chloroperbenzoic acid and RuCl₃.H₂O/periodic acid (H₅IO₆). The use of sterically hindered oxidizing reagents assists in the isolation of the sulfoxide (i.e. compounds of Formula V, where x=1). An example of such an oxidizing reagent is camphorsulfonyl oxaziridine (available as pure enantiomers which can lead to the formation of enantiomerically enriched sulfoxides).

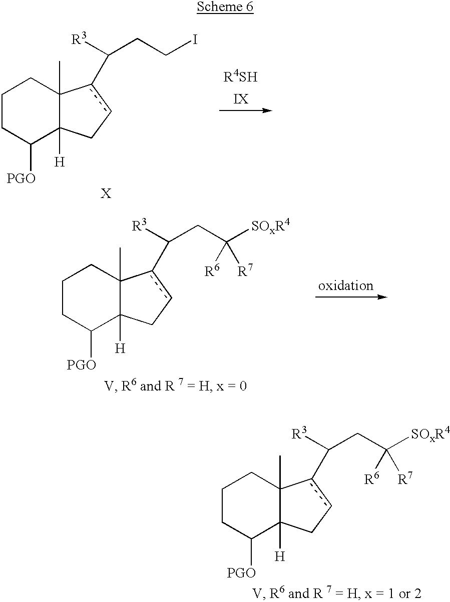

Compounds of Formula V, wherein R³, R⁴ and [symbol] are as defined in Formula I, x is 1 or 2, R⁷ and R⁸ are both H and PG is a suitable protecting group, may alternatively be prepared from aldehyde XI as shown in Scheme 7. Therefore, a compound of Formula VII, wherein R⁴ is as defined in Formula I, R⁶ and R⁷ are both H, and x is 1 or 2, is first treated with a strong base, such as an alkyl lithium, such as n-butyl lithium, under inert conditions and, in the presence of hexamethyl phosphoramide (HMPA), for example, or N,N,N¹,N¹-tetramethylethylenediamine (TMEDA), to generate the corresponding anion, which is then reacted with a compound of Formula XI, wherein R³ and [symbol] are as defined in Formula I and PG is a suitable protecting group, under anhydrous conditions at temperatures in the range of about −60° C. to about −90° C., suitably at about −78° C. The resulting α,β-unsaturated sulfone may then be hydrogenated, for example, in the presence of H₂ over palladium on carbon, to provide compounds of Formula V, wherein R³, R⁴ and [symbol] are as defined in Formula I, x is 1 or 2, R⁷ and R⁸ are both H and PG is a suitable protecting group.

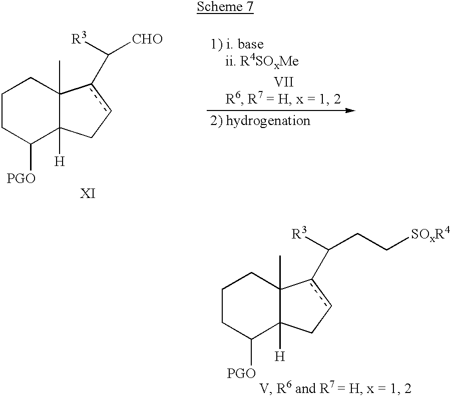

Aldehydes of Formula XI, wherein R³ and [symbol] are as defined in Formula I and PG is a suitable protecting group may be prepared using standard chemistries, for example as shown in Scheme 8. The alcohol groups of compounds of Formula XII, wherein R³ and [symbol] are as defined in Formula I, may be selectively protected to form compounds of Formula XIII, wherein PG and PG are the protecting groups for the primary and secondary alcohol groups respectively, using standard chemistries (see “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 1991.). The primary protected alcohol tosylate of compounds of Formula XII may then be selectively oxidized directly to the corresponding aldehyde XI, for example, in the presence of sodium hydrogen carbonate in a polar aprotic solvent such as dimethlysulfoxide (DMSO) at an elevated temperature, for example 150° C., using a procedure described in Kornblum, et al. J. Am. Chem. Soc. 1959, 81:4113-4116.

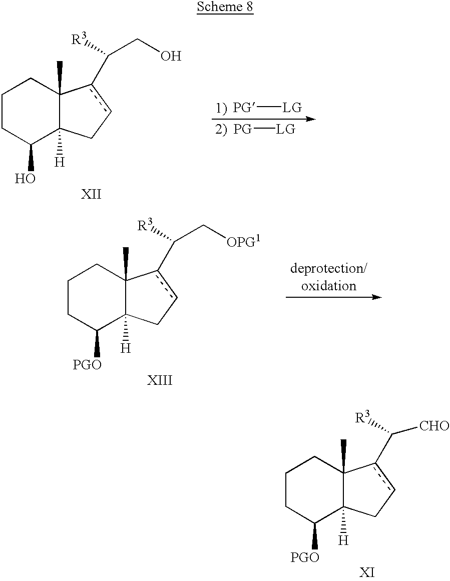

The preparation of compounds of Formula VI, wherein R and [symbol] are as defined in Formula I, and PG is a suitable protecting group, is known in the art. Therefore compounds of Formula VI may be prepared as described in Posner, G. H. et al. J. Med. Chem 0.1992, 42, 3425-3435, the contents of which are incorporated herein by reference.

The preparation of compounds of Formula X, wherein R³ and [symbol] are as defined in Formula I, and PG is a suitable protecting group, is known in the art. Therefore compounds of Formula X may be prepared as described in Posner, G. H. et al. J. Med. Chem 0.1992, 42, 3425-3435; in Jaekyoo Lee, Ph.D. Thesis, 1997, Johns Hopkins University; or in Posner G. H. et al. U.S. Pat. No. 6,380,408, the contents of which are incorporated herein by reference.

The preparation of compounds of Formula IV, wherein R¹, R² and R⁵ are as defined in Formula I is known in the art. Therefore compounds of Formula IV, wherein R¹ and R² are as define in Formula I and both R⁵'s together form ═CH₂, may be prepared as described in Posner, G. H. et al. J. Med. Chem. 1992, 35, 3280-3287, the contents of which are incorporated herein by reference. Compounds of Formula IV, wherein R¹ and R² are as define in Formula I and both R⁵'s are is H, may be prepared as described in Hilpert, H. and Wirz, B. Tetrahedron 2001, 57, 681-694, the contents of which are incorporated herein by reference.

The preparation of compounds of Formula XII, where R³ and [symbol] are as defined in Formula I is known. Therefore compounds of Formula XII, where R³ and [symbol] are as defined in Formula I, may be prepared as described in Posner, G. H. et al. J. Org. Chem. 1997, 62, 3299-3314, the contents of which are incorporated herein by reference.

The preparation of enantiomerically pure compounds of Formula I and or II, may be accomplished by using enantiomerically pure compounds of Formula III and IV in the reaction shown in Scheme I. In this reaction, a mixture of the 1α,3β and 1β,3α diasteromers is typically obtained, with the 1α,3β diastereomer as the major product. These diasteromers may be separated using chromatography, for example using high performance liquid chromatography (HPLC).

In some cases the chemistries outlined above may have to be modified, for instance by use of protective groups, to prevent side reactions due to reactive groups, such as reactive groups attached as substituents. This may be achieved by means of conventional protecting groups, for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 1991.

The formation of a desired compound salt is achieved using standard techniques. For example, the neutral compound is treated with an acid or base in a suitable solvent and the formed salt is isolated by filtration, extraction or any other suitable method.

The formation of solvates of the compounds of the invention will vary depending on the compound and the solvate. In general, solvates are formed by dissolving the compound in the appropriate solvent and isolating the solvate by cooling or using an antisolvent. The solvate is typically dried or azeotroped under ambient conditions.

Prodrugs of the compounds of the invention may be conventional esters formed with available hydroxy, thiol, amino or carboxyl group. For example, when R¹ and/or R² is OH and/or R⁴ is substituted with one or more OH or NH₂ in a compound of the invention, it may be acylated using an activated acid in the presence of a base, and optionally, in inert solvent (e.g. an acid chloride in pyridine). Also, when R¹ is substituted with one or more C(O)OH in a compound of the invention, an ester may be formed by activation of the hydroxyl group of the acid and treatment with the appropriate alcohol in the presence of a base in an inert solvent. Some common esters which have been utilized as prodrugs are phenyl esters, aliphatic (C₈-C₂₄) esters, acyloxymethyl esters, carbamates and amino acid esters.

A radiolabeled compound of the invention may be prepared using standard methods known in the art. For example, tritium may be incorporated into a compound of the invention using standard techniques, for example by hydrogenation of a suitable precursor to a compound of the invention using tritium gas and a catalyst. Alternatively, a compound of the invention containing radioactive iodo may be prepared from the corresponding trialkyltin (suitably trimethyltin) derivative using standard iodination conditions, such as [¹²⁵I] sodium iodide in the presence of chloramine-T in a suitable solvent, such as dimethylformamide. The trialkyltin compound may be prepared from the corresponding non-radioactive halo, suitably iodo, compound using standard palladium-catalyzed stannylation conditions, for example hexamethylditin in the presence of tetrakis(triphenylphosphine) palladium (0) in an inert solvent, such as dioxane, and at elevated temperatures, suitably 50-100° C.

IV. Uses

As hereinbefore mentioned, novel compounds of the Formula I have been prepared. Accordingly, the present invention includes all uses of the compounds of the invention including their use in therapeutic methods and compositions for modulating cell proliferation, their use in diagnostic assays and their use as research tools.

Inhibiting catabolism of calcitriol is expected to lengthen the biological lifetime of this hormone and thus to allow smaller amounts of it to be used for effective human chemotherapy; such smaller dosing is expected to avoid, or at least to minimize, the hypercalcemic toxicity associated with medicinal use of calcitriol. Selectively inhibiting the cytochrome P450 enzymatic pathway, through which calcitriol is catabolized (mainly via C-24 hydroxylation), is one important way to prolong the lifetime of this hormone. Therefore, the compounds of Formula I were tested in vitro, using a standard protocol, for their ability to inhibit specifically CYP24, responsible for 24-hydroxylation of calcitriol. Antimycotic ketoconazole, a drug used clinically for chemotherapy of human prostate cancer (Trachtenberg, J. et al. J. Urol. 1984, J32, 61-63), was used as a control standard for inhibition of CYP24. Compounds of the invention have been shown to selectively inhibit the CYP24 (see Table 1).

In standard hypercalcemia assays, compound Ia did not increase the levels of calcium in the urine at concentrations that were 20-fold higher than the concentration of calcitriol (1α,25-dihydroxy vitamin D₃) that provided an increase in the calcium levels in the urine. Compound 1(u) was also tested and found to be strongly non-calcemic.

The compounds of the invention are CYP24 modulators and are useful in modulating CYP24 activity, including the inhibition of CYP24 activity, for the treatment of various conditions such as all cell proliferative disorders. Accordingly, the invention provides a method of modulating CYP24 activity by administering an effective amount of a compound of the invention to a cell or animal in need thereof. In a further aspect, the invention provides a method of inhibiting CYP24 activity by administering an effective amount of a compound of the invention to a cell or animal in need thereof.

By selectively modulating CYP24, the enzyme that metabolizes 1α,25-dihydroxy vitamin D₃, the levels of 1α,25-dihydroxy vitamin D₃ may also be modulated. Diseases that benefit from a modulation of the levels of 1α,25-dihydroxy vitamin D₃ can therefore be treated using a modulator of CYP24. By acting preferentially on CYP24, side effects caused by interaction with other enzymes and receptors may be reduced. Accordingly, the present invention provides a method for treating diseases which benefit from a modulation of the levels of 1α,25-dihydroxy vitamin D₃ comprising administering an effective amount of a compound of the invention to a cell or animal in need thereof. The invention also includes the use of a compound of the invention to modulate the levels of 1α,25-dihydroxy vitamin D₃. Further, the invention includes a use of a compound of the invention to prepare a medicament to modulate the levels of 1α,25-dihydroxy vitamin D₃.

Inhibition of CYP24, should inhibit the catabolism 1α,25-dihydroxy vitamin D₃. Therefore, in a preferred embodiment, the present invention provides a method for treating diseases which benefit from inhibiting the catabolism of 1α,25-dihydroxy vitamin D₃ comprising administering an effective amount of a compound of the invention to a cell or animal in need thereof. The invention also includes the use of a compound of the invention to inhibit the catabolism of 1α,25-dihydroxy vitamin D₃. Further, the invention includes a use of a compound of the invention to prepare a medicament to inhibit the metabolism of 1α,25-dihydroxy vitamin D₃.

Other diseases which may benefit for a modulation in the levels of 1α,25-dihydroxy vitamin D₃ include, but are not limited to:

i. in the parathyroid—hyper- and hypo-parathyroidism, Osudohypoparathyroidism, Secondary hyperparathyroidism;

ii. in the pancreas—diabetes;

iii. in the thyroid—medullary carcinoma;

iv. in the skin—psoriasis, wound healing;

v. in the lung—sarcoidosis and tuberculosis;

vi. in the kidney—chronic renal disease, hypophosphtatemic VDRR, vitamin D dependent rickets;

vii. in the bone—anticonvulsant treatment, fibrogenisis imperfecta ossium, osteitits fibrosa cystica, osteomalacia, osteporosis, osteopenia, osteosclerosis, renal osteodytrophy, rickets;

viii. in the intestine—glucocorticoid antagonism, idopathic hypercalcemia, malabsorption syndrome, steatorrhea, tropical sprue.

In one aspect, the present invention provides a method for modulating cell proliferation comprising administering an effective amount of a compound of the invention to a cell or animal in need thereof. Preferably, the invention provides a method of inhibiting cell proliferation comprising administering an effective amount of a compound of the invention to a cell or animal in need thereof. The present invention also includes a use of a compound or composition of the invention in order to inhibit cell proliferation, preferably cancer cell proliferation. The present invention further includes a use of a compound or a composition of the invention to prepare a medicament to inhibit cell proliferation, preferably cancer cell proliferation. In particular, the method of the invention is useful in inhibiting the proliferation of abnormal but not normal cells. Abnormal cells include any type of cell that is causative of or involved in a disease or condition and wherein it is desirable to modulate or inhibit the proliferation of the abnormal cell to treat the disease or condition. Examples of abnormal cells include malignant or cancerous cells as well as cell that over-proliferate in inflammatory conditions such as psoriasis. Preferably, the cell proliferative disorder is cancer, in particular cancer of the breast, prostate and lung.

While the compounds of the invention may act by modulating CYP24 activity, one of skill in the art will appreciate that other modes or mechanisms of action for the compounds of the invention are possible.

One skilled in the art can determine which compounds of the invention would have therapeutic utility, for example, in inhibiting cell proliferation in any type of cancer or cell proliferative disorder. Compounds may be examined for their efficacy in inhibiting cell growth in cell proliferation assays such as inhibition of growth of murine keratinocyte cells (cell line PE) and for the inhibition of TPA-induced ornithine decarboxylase (ODC) activity as described in U.S. Pat. No. 5,830,885, the contents of which are incorporated herein by reference.

In addition to cancer, the compounds of the invention are useful in treating other conditions involving aberrant or abnormal cell proliferation. Other cell proliferative disorders that may be treated by the present invention include inflammatory diseases, allergies, autoimmune disease, graft rejection, psoriasis, restenosis, artherosclerosis, and any other disorder wherein it is desirable to inhibit, prevent or suppress cell growth. Compounds of the invention may be tested for their efficacy in a particular cell proliferation disorder using assays and techniques known to those of skill in the art. For example, the following references provide assays for various conditions: Rheumatoid Arthritis: “Regulation of IL-15—Simulated TNF-alpha Production by Rolipram”, Journal of Immunology (1999) volume 163 page 8236 by C. S. Kasyapa et al.; Allergy: “A novel Lyn-Binding Peptide Inhibitor Blocks Eosinophil Differentiation, Survival, and Airway eosinophilic inflammation”. Journal of Immunology (1999) volume 163 page 939 by T. Adachi et al.; Psoriasis: Journal of Immunology (2000) volume 165 page 224 “Inhibition of Keratinocyte apoptosis by IL-15: a new parameter in the pathegenosis of psoriasis” by R. Ochert; and Psoriasis: International Archives of allergy and Immunology (2000) Volume 123 page 275. “T-cell receptor mimic peptides and their potential application in T-cell mediated disease” by A. H. Enk.

The compounds of the invention are preferably formulated into pharmaceutical compositions for administration to human subjects in a biologically compatible form suitable for administration in vivo. Accordingly, in another aspect, the present invention provides a pharmaceutical composition comprising a compound of the invention in admixture with a suitable diluent or carrier.

The compositions containing the compounds of the invention can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of the invention may be used in the form of the free base, in the form of salts, solvates and as hydrates. All forms are within the scope of the invention. Acid addition salts may be formed and provide a more convenient form for use; in practice, use of the salt form inherently amounts to use of the base form. The acids which can be used to prepare the acid addition salts include preferably those which produce, when combined with the free base, form pharmaceutically acceptable salts, that is, salts whose anions are non-toxic to the animal organism in pharmaceutical doses of the salts, so that the beneficial properties inherent in the free base are not vitiated by side effects ascribable to the anions. Although pharmaceutically acceptable salts of the basic compounds are preferred, all acid addition salts are useful as sources of the free base form even if the particular salt per se is desired only as an intermediate product as, for example, when the salt is formed only for the purposes of purification and identification, or when it is used as an intermediate in preparing a pharmaceutically acceptable salt by ion exchange procedures.

In accordance with the methods of the invention, the described compounds or salts or solvates thereof may be administered to a patient in a variety of forms depending on the selected route of administration, as will be understood by those skilled in the art. The compositions of the invention may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A compound of the invention thereof may be orally administered, for example, with an inert diluent or with an assimilable edible carder, or it may be enclosed in hard or soft shell gelatin capsules, or it may be compressed into tablets, or it may be incorporated directly with the food of the diet. For oral therapeutic administration, the compound of the invention may be incorporated with excipient and used in the form of ingestible tablets, buccal tablets, troches, capsules, elixirs, suspensions, syrups, wafers, and the like.

A compound of the invention may also be administered parenterally. Solutions of a compound of the invention can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (1990-18th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The compounds of the invention may be administered to an animal alone or in combination with pharmaceutically acceptable carriers, as noted above, the proportion of which is determined by the solubility and chemical nature of the compound, chosen route of administration and standard pharmaceutical practice.

The dosage of the compounds and/or compositions of the invention can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The compounds of the invention may be administered initially in a suitable dosage that may be adjusted as required, depending on the clinical response. For ex vivo treatment of cells over a short period, for example for 30 minutes to 1 hour or longer, higher doses of compound may be used than for long term in vivo therapy.

The compounds of the invention can be used alone or in combination with other agents that modulate CYP24 activity or in combination with other types of treatment (which may or may not modulate CYP24) for cell proliferative disorders or other disorders that benefit from a modulation in the levels of 1α,25-dihydroxy vitamin D₃ and/or an inhibition of the catabolism of 1α,25-dihydroxy vitamin D₃. Preferably the compounds of the invention may be administered in combination with 1α,25-dihydroxy vitamin D₃ (calcitriol) or other vitamin D receptor agonists. Inhibiting catabolism of vitamin D receptor agonists is expected to lengthen the biological lifetime or efficacy of these therapies and thus to allow smaller amounts of the drug to be used for effective human chemotherapy; such smaller dosing is expected to avoid, or at least to minimize, the hypercalcemic toxicity associated with medicinal use of calcitriol or other vitamin D receptor agonists. The present invention therefore provides a method of increasing the efficacy of a vitamin D receptor agonist, preferably 1α,25-dihydroxy vitamin D₃ (calcitriol), comprising co-administering an effective amount of a compound of the invention and an effective amount of the vitamin D receptor agonist, preferably 1α,25-dihydroxy vitamin D₃ (calcitriol). Further the invention includes the a of a compound of the invention to increase the efficacy of a vitamin D receptor agonist, preferably 1α,25-dihydroxy vitamin D₃ (calcitriol) and a use of a compound of the invention to prepare a medicament to increase the efficacy of a vitamin D receptor agonist, preferably 1α,25-dihydroxy vitamin D₃ (calcitriol).

The general treatments are based on the cancer type and do not specifically target CYP24 activity. In a particular aspect of the present invention, the compounds of the invention may be used in combination with other therapies and therapeutics to treat dermatological disorders, bone disorders, cancer and osteoporosis.

In addition to the above-mentioned therapeutic uses, the compounds of the invention are also useful in diagnostic assays, screening assays and as research tools.

In diagnostic assays the compounds of the invention may be useful in identifying or detecting a cell proliferative disorder. In such an embodiment, the compounds of the invention may be radiolabelled (as hereinbefore described) and contacted with a population of cells. The presence of the radiolabelled on the cells may indicate a cell proliferative disorder.

In screening assays, the compounds of the invention may be used to identify other compounds that modulate cell proliferation or CYP24 activity. As research tools, the compounds of the invention may be used in receptor binding assays and assays to study the localization of CYP24. In such assays, the compounds may also be radiolabelled.

The following non-limiting examples are illustrative of the present invention:

### EXAMPLES

Materials and Methods

Unless otherwise noted, all reactions were performed in oven-dried glassware stirred under an atmosphere of ultra-high-purity argon. THF was distilled from Nalbenzophenone ketyl and CH₂Cl₂ distilled from CaH₂ immediately prior to use. Organolithiums were titrated prior to use following known methods (Suffert, J. J. Org. Chem. 1989, 54, 509-510). All other reagents were used as received from commercial suppliers. Analytical TLC analysis was conducted on precoated glass-backed silica gel plates (Merck Kieselgel 60 F₂₅₄, 250 mm thickness) and visualized with p-anisaldehyde or KMnO₄ stains. Column chromatography was performed using short path silica gel (particle size<230 mesh) or flash silica gel (particle size 230-400 mesh). Preparative-plate chromatography was performed using silica-gel-coated glass preparative plates (500-1000 μm) from Analtech and analyzed by UV. HPLC was carried out using a Rainin HPLX™ system equipped with two 25-mL/min preparative pump heads using (1) a Chiral Technologies CHIRALCEL® OJ 10-mm×250-mm (semipreparative) column packed with cellulose tris(4-methylbenzoate) on a 10 μm silica-gel substrate or (2) a Phenomenex LUNA™ 10-mm×250-mm (semipreparative) column packed with 110 Å silica gel (5 μm pore size) as C-18-bonded silica and a Rainin Dynamax™ UV-C dual-beam variable-wavelength detector set at 254 nm. Yields are reported for pure products (>95% based on their chromatographic and spectroscopic homogeneity) and are unoptimized. Melting points were determined in open capillaries using a MeI-Temp metal-block apparatus and are uncorrected. Optical rotations were measured at the Na line using a Perkin-Elmer 141 Polarimeter. NMR spectra were obtained on a Varian XL-400 spectrometer operating at 400 MHz for ¹H, 376 MHz for ¹⁹F, and 100 MHz for ¹³C and a Bruker 300 AMX spectrometer operating at 300 MHz for ¹H. Chemical shifts are reported in ppm (δ) and are referenced to CDCl₃ (7.26 ppm for ¹H and 77.0 ppm for ¹³C), tetramethylsilane (TMS, 0.00 ppm for ¹H), and CFCl₃ (0.00 ppm for ¹⁹F). IR spectra were obtained using a Perkin Elmer 1600 Series FT-IR instrument. HRMS were obtained at the mass spectrometry facility at the Ohio State University on a Micromass QTOF Electrospray mass spectrometer. Elemental analyses were performed by Atlantic Microlab Inc., Norcross, Ga.

### Example 1

### General Procedure for the Preparation of Aryl Methyl Sulfones VII

To an ice-cold solution containing the appropriate aryl methyl sulfide (6.00 mmol) in MeOH (24.0 mL) was added oxone® (9.00 mmol) as a solution in H₂O (20.0 mL) dropwise via addition funnel. The resulting cloudy slurry was stirred at room temperature overnight, diluted with water, and extracted with CHCl₃ (3×). The combined organics were washed with water and brine, dried over Na₂SO₄, and concentrated under reduced pressure to give essentially quantitative recovery of the aryl methyl sulfones VII (a-c) as crystalline solids.

a) Methyl-(4-methoxyphenyl) sulfone. According to the general procedure for the preparation of aryl methyl sulfones described above, 1-methanesulfanyl-4-methoxy-benzene (1.00 g, 6.48 mmol) gave 1.20 g (99%) of the title compound as a white solid: mp 114-115° C. (lit. mp 115° C., Helv. Chim. Acta. 1999, 82, 372-388); ¹H NMR (400 MHz, CDCl₃) δ 7.89-7.83 (dt, J=9.5, 2.8, 2.2 Hz, 2H), 7.04-6.99 (dt, J=9.5, 2.8, 2.2 Hz, 2H), 3.88 (s, 3H), 3.02 (s, 3H); ¹³C NMR (100 MHz, CDCl₃) δ 163.6, 132.3, 129.5, 114.5, 55.7, 44.8; IR (neat) 3020, 3010, 2982, 1575, 1412, 1323, 1293, 1142, 1092, 1023, 835, 766, 544, 528 cm⁻¹; Anal. Calcd for C₈H₁₀O₃S: C, 51.60; H, 5.41. Found: C, 51.64; H, 5.43.

b) Methyl-(4-nitrophenyl) sulfone. According to the general procedure for the preparation of aryl methyl sulfones described above, 1-methanesulfanyl-4-nitrobenzene (1.00 g, 5.41 mmol) gave 1.19 g (100%) of the title compound as a yellow solid: mp 137-139° C.; ¹H NMR (400 MHz, CDCl₃) δ 8.45-8.40 (dt, J=9.0, 2.5 Hz, 2H), 8.19-8.14 (dt, J=9.0, 2.5 Hz, 2H), 3.12 (s, 3H).

c) Methyl-(4-trifluoromethylphenyl) sulfone. According to the general procedure for the preparation of aryl methyl sulfones described above, 1-methanesulfanyl-4-trifluoromethylbenzene, synthesized from 4-chloro-1-trifluoromethyl benzene and sodium methanethiolate as described in Cabiddu, M. G. et al., J. Organometallic Chem. 1997, 531, 125-140. (1.07 g, 5.57 mmol) gave 1.21 g (97%) of the title compound as a white solid: mp 100-101° C.; ¹H NMR (300 MHz, CDCl₃) δ 8.13-8.07 (d, J=11.1 Hz, 2H), 7.89-7.83 (d, J=11.1 Hz, 2H), 3.09 (s, 3H); ¹³C NMR (100 MHz, CDCl₃) δ 143.9, 135.4 (d, J=32.6 Hz), 128.1, 126.5 (d, J=0.8 Hz), 123.0 (d, J=123.0 Hz), 44.3; ¹⁹F NMR (375 MHz, CDCl₃, CFCl₃) δ-69.4 (m).

### Example 2

### Preparation of Methyl (p-acetalphenyl) Sulfone

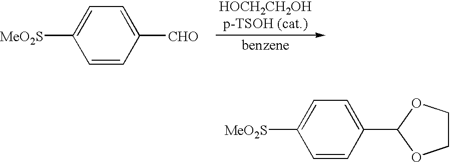

A mixture of 4-methylsulphonyl benzaldehyde (750 mg, 3.87 mmol, 95% purity) and ethylene glycol (0.9 mL, 16.0 mmol) in benzene (10 mL) in the presence of catalytic amount of p-TsOH was refluxed for 6.5 h. After benzene was distilled off, the residue was dissolved into EtOAc. The organic layer was washed with brine, saturated aq. NaHCO₃, and brine again, dried over MgSO₄, filtered, concentrated to afford 781.9 mg (88%) of a crude product which was directly used for the next reaction without further purification. R_{f}0.37 (1:1-EtOAc:Hex); ¹H NMR (400 MHz, CDCl₃) δ 7.95-7.98 (m, 2H), 7.68-7.71 (m, 2H), 5.89 (s, 1H), 4.05-4.15 (m, 4H), 3.05 (s, 3H).

### Example 3

### (Triethylsilyl)-oxy-aryl Sulfones V

(a) (Triethylsilyl)-oxy-phenyl Sulfone

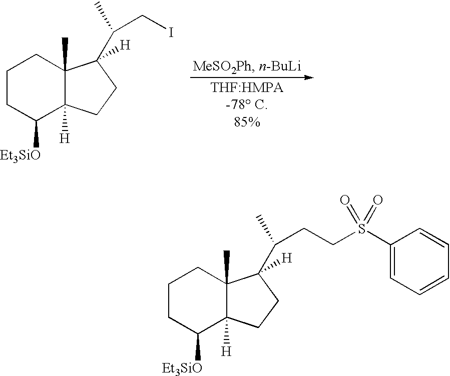

To a cold (−78° C.) solution of methyl phenyl sulfone (125 mg, 0.802 mmol) in THF (2.25 mL) was added a solution of n-BuLi (556 μL, 0.802 mmol, 1.44 M in hexanes) dropwise via syringe. After 15 min, HMPA (0.1-0.2 mL) was added and the solution was stirred for an additional 15 min at −78° C. A precooled (−78° C.) solution of the (+)-triethylsilyl iodide (Posner, G. H.; Crawford, K. R. unpublished results, 100 mg, 0.229 mmol) in THF (0.75 mL) was added slowly via cannula. The reaction mixture was then warmed to room temperature. The reaction was quenched with H₂O, extracted with Et₂O (3×), washed with brine, dried over MgSO₄, and concentrated to a crude solid that was purified by chromatography (5→20% EtOAc/hexanes) to give 91 mg (85%) of the title compound as a colorless oil: [α]_{D}²⁵+36.7 (c 4.3, CHCl₃); ¹H NMR (400 MHz, CDCl₃) δ 7.92-7.85 (m, 2H), 7.67-7.59 (tt, J=7.4, 1.5 Hz, 1H), 7.59-7.50 (m, 2H), 4.043.96 (m, 1H), 3.16-3.04 (m, 1H), 3.02-2.91 (m, 1H), 0.92 (t, J=8.0 Hz, 9H), 0.84 (s, 3H), 0.83 (d, J=6.8 Hz, 3H), 0.52 (q, J=8.0 Hz, 6H); ¹³C NMR (100 MHz, CDCl₃) δ 139.2, 133.5, 129.1, 128.0, 69.2, 55.9, 53.6, 52.9, 42.1, 40.6, 34.4, 34.2, 28.2, 26.9, 22.8, 18.2, 17.5, 13.4, 6.9, 4.9; IR (neat) 2949, 2912, 2873, 1446, 1317, 1306, 1234, 1148, 1087, 1021, 803, 740, 724, 689 cm⁻¹; HRMS: calcd for C₂₆H₄₄O₅SSi+Na, 487.2678, found 487.2672.

In a like manner, the following additional compounds were prepared:

(b) (+)-(Triethylsilyl)-oxy-(4-fluorophenyl) Sulfone: By replacing methyl phenyl sulfone with methyl (4-fluorophenyl) sulfone. The crude mixture was purified by flash chromatography (EtOAc:Hex=1:15 to 1:13) to afford 78 mg (85%) of C24-p-fluorophenyl sulfone. R_{f}0.37 (1:9-EtOAc:Hex); [α]_{D}²⁶+36.0 (c 1.11, CHCl₃); ¹H NMR (400 MHz, CDCl₃) δ 7.90-7.94 (m, 2H), 7.23-7.27 (m, 2H), 4.01 (d, J=2.4 Hz, 1H), 3.12 (ddd, J=4.0, 12.0, 13.6 Hz, 1H), 2.98 (ddd, J=4.8, 11.6, 14.0 Hz, 1H), 1.70-1.88 (m, 2H), 1.61-1.68 (m, 2H), 1.40-1.57 (m, 3H), 1.02-1.38 (m, 8H), 0.93 (t, J=7.6 Hz, 9H), 0.86 (s, 3H), 0.85 (d, J=7.2 Hz, 3H), 0.54 (q, J=8.0 Hz, 6H); ¹³C NMR (100 MHz, CDCl₃) δ 165.7 (d, J=255.1), 135.2 (d, J=3.2), 130.9 (d, J=9.6), 116.5 (d, J=22.3), 69.2, 55.9, 53.8, 52.9, 42.1, 40.6, 34.5, 34.2, 28.3, 27.0, 22.8, 18.3, 17.6, 13.5, 6.9, 4.9; IR (thin film) 2950, 2876, 1592, 1494, 1321, 1289, 1236, 1148, 1087 cm⁻¹; HRMS calc'd for [M+Na]: 505.2578 for C₂₆H₄₃FO₃SSiNa. found: 505.2561.

(c) (+)-Triethylsilyl)-oxy-(4-chlorophenyl) Sulfone: By replacing methyl phenyl sulfone with methyl (4-chlorophenyl) sulfone. The crude mixture was purified by flash chromatography (EtOAc:Hex=1:12 to 1:7) to afford 87.5 mg (96%) of C24-p-chlorophenyl sulfone. R_{f}0.35 (1:9-EtOAc:Hex); [α]_{D}²⁶+37.8 (c 0.91, CHCl₃); ¹H NMR (400 MHz, CDCl₃) δ 7.82-7.86 (m, 2H), 7.53-7.56 (m, 2H), 4.01 (m, 1H), 3.11 (ddd, J=4.8, 12.0, 13.6 Hz, 1H), 2.97 (ddd, J=4.8, 11.6, 14.0 Hz, 1H), 1.76-1.90 (m, 2H), 1.60-1.69 (m, 2H), 1.40-1.58 (m, 3H), 1.02-1.36 (m, 8H), 0.93 (t, J=8.0 Hz, 9H), 0.86 (s, 3H), 0.85 (d, J=6.4 Hz, 3H), 0.54 (q, J=8.0 Hz, 6H); ¹³C NMR (100 MHz, CDCl₃) δ 140.3, 137.6, 129.5, 69.1, 55.9, 53.7, 52.9, 42.1, 40.6, 34.4, 34.2, 28.2, 27.0, 22.8, 18.2, 17.6, 13.4, 6.9, 4.9; IR (thin film) 2950, 2875, 1312, 1150, 1088 cm⁻¹; HRMS calc'd for [M+Na]: 521.2288 for C₂₆H₄₃ClO₃SSiNa. found: 521.2275.

(d) (+)-(Triethylsilyl)-oxy-(4-methylphenyl) Sulfone: By replacing methyl phenyl sulfone with methyl (4-methyphenyl) sulfone. The crude mixture was purified by flash chromatography (EtOAc:Hex=1:12 to 1:10) to afford 84.6 mg (93%) of C24-p-tolyl sulfone. R_{f}0.23 (1:9-EtOAc:Hex); [α]_{D}²⁶+36.9 (c 0.96, CHCl₃); ¹H NMR (400 MHz, CDCl₃) δ 7.76-7.78 (m, 2H), 7.33-7.36 (m, 2H), 3.99 (d, J=2.4 Hz, 1H), 3.09 (ddd, J=4.0, 12.0, 13.6 Hz, 1H), 2.95 (ddd, J=4.8, 11.6, 14.0 Hz, 1H), 2.44 (s, 3H), 1.72-1.87 (m, 3H), 1.62-1.68 (m, 2H), 1.38-1.60 (m, 3H), 1.00-1.35 (m, 7H), 0.92 (t, J=7.6 Hz, 9H), 0.84 (s, 3H), 0.82 (d, J=6.4 Hz, 3H), 0.52 (q, J=7.6 Hz, 6H); ¹³C NMR (100 MHz, CDCl₃) δ 144.4, 136.2, 129.8, 128.0, 69.2, 55.9, 53.7, 52.9, 42.1, 40.6, 34.5, 34.2, 28.3, 27.0, 22.8, 21.6, 18.2, 17.6, 13.4, 6.9, 4.9; IR (thin film) 2950, 2875, 1598, 1456, 1316, 1148, 1088 cm⁻¹; HRMS [M+Na] calc'd 501.2829 for C₂₇H₄₆O₃SSiNa. found: 501.2810.

(e) (+)-(Triethylsilyl)-oxy-(3,4-dichlorophenyl) Sulfone: By replacing methyl phenyl sulfone with methyl (3,4-dichlorophenyl) sulfone. The crude mixture was purified by flash chromatography (EtOAc:Hex=1:12) to afford 65.6 mg (66%) of C24-3,4-dichlorophenyl sulfone. R_{f}0.38 (1:9-EtOAc:Hex); [α]_{D}²⁶+32.2 (c 1.03, CHCl₃); ¹H NMR (400 MHz, CDCl₃) δ 8.00 (d, J=2.0 Hz, 1H), 7.73 (dd, J=2.0, 8.4 Hz, 1H), 7.66 (d, J=8.4 Hz, 1H), 4.02 (d, J=2.4 Hz, 1H), 3.13 (ddd, J=4.8, 12.0, 14.0 Hz, 1H), 2.99 (ddd, J=4.8, 11.6, 14.0 Hz, 1H), 1.76-1.90 (m, 3H), 1.62-1.73 (m, 2H), 1.42-1.59 (m, 4H), 1.28-1.38 (m, 3H), 1.14-1.26 (m, 2H), 1.06 (dt, J=3.2, 13.2 Hz, 1H), 0.94 (t, J=8.0 Hz, 9H), 0.87 (s, 3H), 0.86 (d, J=6.0 Hz, 3H), 0.54 (q, J=8.0 Hz, 6H); ¹³C NMR (100 MHz, CDCl₃) δ 139.0, 138.6, 134.0, 131.3, 130.0, 127.1, 69.1, 55.8, 53.7, 52.9, 42.1, 40.6, 34.4, 34.2, 28.1, 27.0, 22.8, 18.2, 17.5, 13.4, 6.9, 4.8; IR (thin film) 2950, 2875, 1455, 1370, 1322, 1156, 1091 cm⁻¹; HRMS [M+Na] calc'd 555.1893 for C₂₆H₄₂Cl₂O₃SSiNa. found: 555.1886.

(f) (+)-(Triethylsilyl)-oxy-p-[3-(tert-Butyldimethylsiloxy)isopentyl]phenyl sulfone: By replacing methyl phenyl sulfone with p-(3-(tert-Butyldimethylsiloxy)isopentyl)phenyl methyl sulfone. The crude mixture was purified by flash chromatography (EtOAc:Hex=1:19 to 1:15) to afford 107.7 mg (94%) of C24-p-[3-(tert-Butyldimethylsiloxy)isopentyl]phenyl sulfone. R_{f}0.39 (1:9-EtOAc:Hex); [α]_{D}²⁶+21.3 (c 0.97, CHCl₃); ¹H NMR (400 MHz, CDCl₃) δ 7.82-7.85 (m, 2H), 7.54-7.57 (m, 2H), 4.00 (m, 1H), 3.11 (ddd, J=4.0, 12.0, 13.6 Hz, 11), 2.98 (ddd, J=4.8, 11.2, 13.6 Hz, 1H), 1.75-1.96 (m, 7H), 1.43-1.66 (m, 5H), 1.24-1.35 (m, 5H), 1.00-1.20 (m, 2H), 1.00 (s, 9H), 0.93 (s, 9H), 0.84 (d, J=6.0 Hz, 3H), 0.84 (s, 3H), 0.63 (t, J=7.2 Hz, 6H), 0.53 (q, J=8.0 Hz, 6H), 0.16 (s, 6H); ¹³C NMR (100 MHz, CDCl₃) δ 152.6, 136.5, 127.6, 126.8, 81.5, 69.2, 55.8, 53.6, 52.9, 42.1, 40.6, 35.65, 35.62, 34.5, 34.2, 28.3, 26.9, 26.2, 22.8, 18.9, 18.3, 17.6, 13.5, 8.2, 6.9, 4.9, −2.1; IR (thin film) 2952, 2877, 1462, 1318, 1256, 1151, 1025, 836, 800, 771 cm⁻¹; HRMS [M+Na] calc'd 687.4269 for C₃₇H₆₈O₄SSi₂Na. found: 687.4293.

(g) (+)-(Triethylsilyl)-oxy-p-Acetalphenyl sulfone: By replacing methyl phenyl sulfone with p-acetalphenyl methyl sulfone (Example 2). The crude mixture was purified by flash chromatography (EtOAc:Hex=1:4) to afford 83.5 mg (74%) of C24-p-acetalphenyl sulfone. R_{f}0.26 (1:4-EtOAc:Hex); [α]_{D}²⁶+33.2 (c 1.10, CHCl₃); ¹H NMR (400 MHz, CDCl₃) δ 7.91-7.93 (m, 2H), 7.67-7.69 (m, 2H), 5.89 (s, 1H), 4.05-4.15 (m, 4H), 4.01 (m, 1H), 3.14 (ddd, J=4.0, 12.0, 13.6 Hz, 1H), 2.97 (ddd, J=4.8, 11.6, 13.6 Hz, 1H), 1.75-1.89 (m, 3H), 1.60-1.70 (m, 2H), 1.40-1.58 (m, 3H), 1.28-1.36 (m, 3H), 1.02-1.21 (m, 4H), 0.93 (t, J=8.4 Hz, 9H), 0.86 (s, 3H), 0.83 (d, J=6.0 Hz, 3H), 0.54 (q, J=8.4 Hz, 6H); ¹³C NMR (100 MHz, CDCl₃) δ 143.8, 139.7, 128.1, 127.2, 102.4, 69.1, 65.4, 55.9, 53.6, 52.9, 42.1, 40.6, 34.4, 34.2, 28.1, 26.9, 22.8, 18.2, 17.5, 13.4, 6.8, 4.8; IR (thin film) 2950, 2876, 1316, 1149, 1085, 1018, 973, 948, 744, 725, 547 cm⁻¹; HRMS [M+Na] calc'd 559.2884 for C₂₉H₄₈O₅SSiNa. found: 559.2930.

In a like manner, the following additional compounds can be prepared:

(h) (Triethylsilyl)-oxy-(4-methoxyphenyl) sulfone: By replacing methyl phenyl sulfone with methyl (4-methoxyphenyl) sulfone (Example 1a);

(i) (Triethylsilyl)-oxy-(4-nitrophenyl) sulfone: By replacing methyl phenyl sulfone with methyl (4-nitrophenyl) sulfone (Example 1b); and

(j) (Triethylsilyl)-oxy-(4-trifluoromethyl phenyl) sulfone: By replacing methylpheny sulfone with methyl (4-trifluoromethyl phenyl) sulfone (Example 1c).

### Example 4

### C,D-Ring Ketones III

(a) (+)-Ketophenyl Sulfone

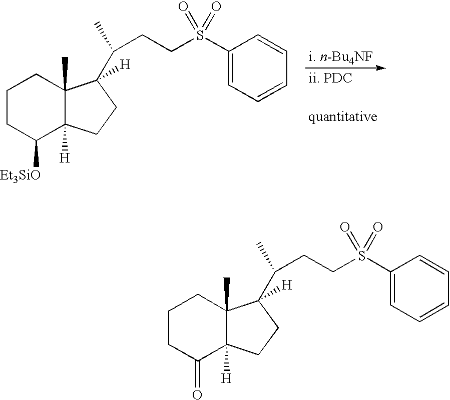

To a solution of (triethylsilyl)-oxyphenyl sulfone (Example 3a, 86 mg, 0.185 mmol) in THF (˜0.7 M) was added a solution of tetrabutylanimonium fluoride (TBAF, 740 μL, 0.740 mmol, 1.0 M in THF). The reaction mixture was stirred for 18 h and concentrated under reduced pressure to a brown syrup. This brown syrup was then dissolved in CH₂Cl₂ and treated with pyridinium dichromate (PDC, 290 mg, 0.555 mmol) and celite® (109 mg) for 12 h. The contents of the flask were then passed through a 1″ plug of silica gel, rinsed with EtOAc (3×), concentrated, and purified by flash chromatography (35→40% EtOAc/hexanes) or preparative-plate chromatography (50% EtOAc/hexanes) to afford pure C,D-ring ketone (67 mg) in quantitative yield as a colorless oil: [α]_{D}²⁵+17.7 (c 4.3, CHCl₃); ¹H NMR (400 MHz, CDCl₃) δ 7.92-7.84 (m, 2H), 7.67-7.60 (tt, J=7.6, 1.7 Hz, 1H), 7.59-7.51 (m, 2H), 3.16-3.04 (m, 1H), 3.03-2.91 (m, 1H), 2.45-2.33 (dd, J=11.4, 7.4 Hz, 1H), 2.29-2.11 (m, 2H), 2.07-1.91 (m, 2H), 0.89 (d, J=6.4 Hz, 3H), 0.52 (s, 3H); ¹³C NMR (100 MHz, CDCl₃) δ 139.0, 133.6, 129.2, 127.9, 61.6, 55.7, 53.4, 49.6, 40.7, 38.7, 34.3, 28.1, 27.1, 23.8, 18.9, 18.3, 12.4; IR (neat) 2956, 2875, 1709, 1446, 1306, 1145, 1086, 747, 690 cm⁻¹; HRMS: calcd for C₂₀H₂₈O₅S+Na, 371.1657, found 371.1664.

In a like manner, the following additional compounds were prepared:

(b) (+)-Keto-(4-fluorophenyl) Sulfone: By replacing (triethylsilyl)-oxyphenyl sulfone with (triethylsilyl)-oxy-(4-fluorophenyl) sulfone (Example 3b). The reaction mixture was directly purified by short path flash chromatography (CH₂Cl₂ then EtOAc:Hex=1:2) to give 50.5 mg (85% for 2 steps) of keto-p-fluorophenyl sulfone. R_{f}0.30 (1:2-EtOAc:Hex); [α]_{D}²⁶+11.5 (c 0.96, CHCl₃); ¹H NMR (400 MHz, CDCl₃) δ 7.90-7.95 (m, 2H), 7.24-7.28 (m, 2H), 3.13 (ddd, J=4.4, 12.0, 13.6 Hz, 1H), 3.00 (ddd, J=4.8, 11.2, 14.0 Hz, 1H), 2.43 (dd, J=7.6, 11.6 Hz, 1H), 2.17-2.32 (m, 2H), 1.97-2.08 (m, 2H), 1.67-1.94 (m, 4H), 1.45-1.60 (m, 4H), 1.39 (q, J=9.2 Hz, 1H), 1.27 (m, 1H), 0.93 (d, J=6.0 Hz, 3H), 0.61 (s, 3H); ¹³C NMR (100 MHz, CDCl₃) δ 211.4, 165.7 (d, J=255.1), 135.1 (d, J=3.0), 130.8 (d, J=9.9), 116.6 (d, J=22.8), 61.6, 55.8, 53.6, 49.7, 40.8, 38.7, 34.4, 28.2, 27.2, 23.8, 18.9, 18.3, 12.4; IR (thin film) 2957, 2876, 1710, 1591, 1494, 1316, 1289, 1232, 1144, 1086 cm⁻¹; HRMS [M+Na] calc'd 389.1557 for C₂₀H₂₇FO₃SNa. found: 389.1547.

(c) (+)-Keto-(4-chlorophenyl) Sulfone: By replacing (triethylsilyl)-oxyphenyl sulfone with (triethylsilyl)-oxy-(4-chlorophenyl) sulfone (Example 3c). The reaction mixture was directly purified by short path flash chromatography (CH₂Cl₂ then EtOAc:Hex=1:2) to give 56.2 mg (82% for 2 steps) of keto-p-chlorophenyl sulfone. R_{f}0.34 (1:2-EtOAc:Hex); [α]_{D}²⁶+12.5 (c 0.97, CHCl₃); ¹H NMR (400 MHz, CDCl₃) δ 7.83-7.86 (m, 2H), 7.54-7.58 (m, 2H), 3.13 (ddd, J=4.8, 12.0, 14.0 Hz, 1H), 3.00 (ddd, J=4.8, 11.2, 14.0 Hz, 1H), 2.43 (m, 1H), 2.17-2.31 (m, 2H), 1.97-2.08 (m, 2H), 1.68-1.94 (m, 4H), 1.22-1.59 (m, 4H), 0.93 (d, J=6.4 Hz, 3H), 0.61 (s, 3H); ¹³C NMR (100 MHz, CDCl₃) δ 211.4, 140.4, 137.5, 129.6, 129.5, 61.7, 55.8, 53.6, 49.7, 40.8, 38.8, 34.4, 28.2, 27.2, 23.8, 18.9, 18.3, 12.4; IR (thin film) 2957, 2876, 1710, 1315, 1149, 1088 cm⁻¹; HRMS [M+Na] calc'd 405.1267 for C₂₀H₂₇ClO₃SNa. found: 405.1263.

(d) (+)-Keto-(4-methylphenyl) Sulfone: By replacing (triethylsilyl)-oxyphenyl sulfone with (triethylsilyl)-oxy-(4-methylphenyl) sulfone (Example 3d). The reaction mixture was directly purified by short path flash chromatography (CH₂Cl₂ then EtOAc:Hex=1:2) to give 57.4 mg (89% for 2 steps) of keto-p-fluorophenyl. R_{f}0.26 (1:2-EtOAc:Hex); [α]_{D}²⁶+9.4 (c 1.15, CHCl₃); ¹H NMR (400 MHz, CDCl₃) δ 7.77-7.79 (m, 2H), 7.36-7.38 (m, 2H), 3.11 (ddd, J=4.4, 12.0, 13.6 Hz, 1H), 2.98 (ddd, J=4.8, 11.2, 13.6 Hz, 1H), 2.46 (s, 3H), 2.42 (dd, J=8.0, 12.0 Hz, 1H), 2.16-2.31 (m, 2H), 1.96-2.08 (m, 2H), 1.66-1.94 (m, 4H), 1.44-1.58 (m, 4H), 1.38 (q, J=9.2 Hz, 1H), 1.25 (m, 1H), 0.92 (d, J=6.4 Hz, 3H), 0.60 (s, 3H); ¹³C NMR (100 MHz, CDCl₃) δ 211.5, 144.6, 136.0, 129.8, 127.9, 61.6, 55.8, 53.5, 49.6, 40.7, 38.7, 34.3, 28.2, 27.1, 23.8, 21.6, 18.9, 18.3, 12.4; IR (thin film) 2956, 2875, 1710, 1314, 1143, 1087 cm⁻¹; HRMS [M+Na] calc'd 385.1808 for C₂₁H₃₀O₃SNa. found: 385.1825.

(e) (+)-Keto-(3,4-dichlorophenyl) Sulfone: By replacing (triethylsilyl)-oxyphenyl sulfone with (triethylsilyl)-oxy-(3,4-dichlorophenyl) sulfone (Example 3e). The reaction mixture was directly purified by short path flash chromatography (CH₂Cl₂ then EtOAc:Hex=1:2) to give 47.3 mg (94% for 2 steps) of keto-3,4-dichlororophenyl sulfone. R_{f}0.39 (1:2-EtOAc:Hex); [α]_{D}²⁶+10.9 (c 0.99, CHCl₃); ¹H NMR (400 MHz, CDCl₃) δ 8.00 (dd, J=0.4, 2.0 Hz, 1H), 7.73 (dd, J=2.0, 8.4 Hz, 1H), 7.67 (dd, J=0.4, 8.4 Hz, 1H), 3.14 (ddd, J=4.8, 11.6, 14.0 Hz, 1H), 3.01 (ddd, J=4.8, 11.2, 14.0 Hz, 1H), 2.44 (dd, J=7.6, 12.0 Hz, 1H), 2.17-2.32 (m, 2H), 1.98-2.09 (m, 2H), 1.69-1.95 (m, 4H), 1.46-1.60 (m, 4H), 1.40 (q, J=9.2 Hz, 1H), 1.29 (m, 1H), 0.94 (d, J=6.4 Hz, 3H), 0.62 (s, 3H); ¹³C NMR (100 MHz, CDCl₃) δ 211.4, 138.85, 138.81, 134.0, 131.4, 130.0, 127.0, 61.7, 55.8, 53.6, 49.7, 40.8, 38.8, 34.5, 28.0, 27.3, 23.8, 18.9, 18.3, 12.4; IR (thin film) 3086, 2957, 2876, 1710, 1455, 1370, 1317, 1150, 1094, 1034, 824, 753, 676, 634 cm⁻¹; HRMS [M+Na] calc'd 439.0872 for C₂₀H₂₆Cl₂O₃SNa. found: 439.0832.

(f) (+)-Keto-p-[3-(tert-Butyldimethylsiloxy)isopentyl]phenyl Sulfone: By replacing (triethylsilyl)-oxyphenyl sulfone with p-[3-(tert-butyldimethylsiloxy)isopentyl]phenyl sulfone (Example 3f). The reaction mixture was directly purified by short path flash chromatography (CH₂Cl₂ then EtOAc:Hex=1:2) to give 75.8 mg (97% for 2 steps) of keto-p-(3-(tert-Butyldimethylsiloxy)isopentyl)phenyl sulfone. R_{f}0.53 (1:2-EtOAc:Hex); [α]_{D}²⁶+3.6 (c 1.64, CHCl₃); ¹H NMR (400 MHz, CDCl₃) δ 7.83-7.85 (m, 2H), 7.55-7.58 (m, 2H), 3.12 (ddd, J=4.0, 12.0, 14.0 Hz, 1H), 3.00 (ddd, J=4.8, 10.8, 13.6 Hz, 1H), 2.41 (dd, J=7.6, 11.2, 1H), 2.16-2.30 (m, 2H), 1.79-2.07 (m, 8H), 1.63-1.74 (m, 2H), 1.45-1.57 (m, 3H), 1.34-1.42 (m, 1H), 1.14-1.28 (m, 2H), 1.00 (s, 9H), 0.92 (d, J=6.8 Hz, 3H), 0.62 (t, J=7.2 Hz, 6H), 0.58 (s, 3H), 0.17 (s, 6H); ¹³C NMR (100 MHz, CDCl₃) δ 211.4, 152.8, 136.3, 127.5, 126.8, 81.4, 61.6, 55.7, 53.4, 49.6, 40.8, 38.7, 35.59, 35.56, 27.1, 26.2, 23.8, 18.9, 18.8, 18.3, 12.4, 8.2, −2.2; IR (thin film) 2956, 1713, 1458, 1315, 1256, 1147, 1062, 836, 798, 771 cm⁻¹; HRMS [M+Na] calc'd 571.3248 for C₃₁H₅₂O₄SSiNa. found: 571.3284

(g) (+)-Keto-p-Acetalphenyl Sulfone: By replacing (triethylsilyl)-oxyphenyl sulfone with (+)-(triethylsilyl)-oxy-p-Acetalphenyl sulfone (Example 3g). The reaction mixture was directly purified by short path flash chromatography (CH₂Cl₂ then EtOAc:Hex=1:1) to give 66 mg (100% for 2 steps) of keto-p-acetalphenyl sulfone. R_{f}0.38 (1:1-EtOAc:Hex); [α]_{D}²⁶+11.1 (c 0.97, CHCl₃); ¹H NMR (400 MHz, CDCl₃) δ 7.91-7.93 (m, 2H), 7.68-7.70 (m, 2H), 5.88 (s, 1H), 4.06-4.15 (m, 4H), 3.12 (ddd, J=4.4, 11.6, 14.0 Hz, 1H), 2.99 (ddd, J=4.8, 11.2, 14.0 Hz, 1H), 2.42 (dd, J=7.6, 12.0 Hz, 1H), 2.17-2.31 (m, 2H), 1.97-2.06 (m, 2H), 1.66-1.94 (m, 4H), 1.44-1.58 (m, 3H), 1.37 (q, J=9.6 Hz, 1H), 0.92 (d, J=6.4 Hz, 3H), 0.60 (s, 3H); ¹³C NMR (100 MHz, CDCl₃) δ 211.4, 143.9, 139.6, 128.0, 127.3, 102.3, 65.4, 61.6, 55.8, 53.5, 49.6, 40.7, 38.7, 34.4, 28.1, 27.1, 23.8, 18.9, 18.2, 12.4; IR (thin film) 2957, 2879, 1709, 1381, 1145, 1086, 943, 754, 549 cm⁻¹; HRMS [M+Na] calc'd 443.1863 for C₂₃H₃₂O₅SNa. found: 443.1843.

In a like manner, the following additional compounds can be prepared:

(h) Keto-(4-methoxyphenyl) sulfone: By replacing (triethylsilyl)-oxyphenyl sulfone with (triethylsilyl)-oxy-(4-methoxyphenyl) sulfone (Example 3h);

(i) Keto-(4-nitrophenyl) sulfone: By replacing (triethylsilyl)-oxyphenyl sulfone with (triethylsilyl)-oxy-(4-nitrophenyl) sulfone (Example 3i); and

(j) Keto-(4-trifluoromethylphenyl) sulfone: By replacing (triethylsilyl)-oxyphenyl sulfone with (triethylsilyl)-oxy-(4-trifluoromethylphenyl) sulfone (Example 3j).

### Example 5

### 24-Phenyl Sulfone Vitamin-D₃ Analogs (1)

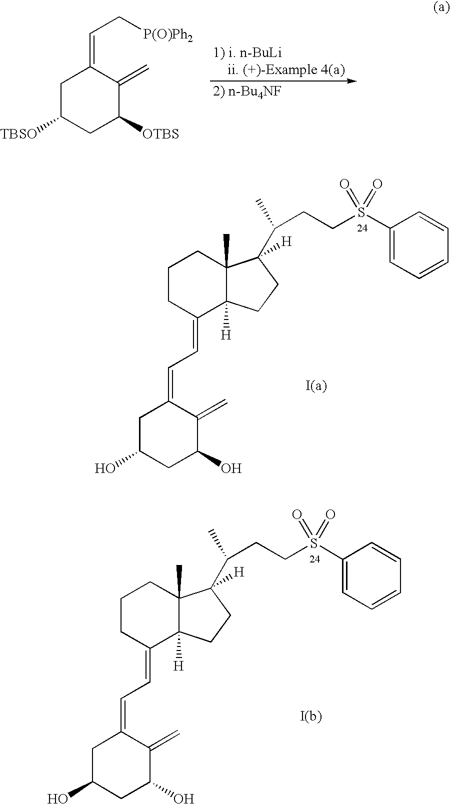

Prior to reaction, the phosphine oxide (Posner, G. H. et al. J. Med. Chem. 1992, 35, 3280-3287) and C,D-ring ketone of Example 3a were azeotrophically dried with benzene and left under vacuum for 48 h. A solution of n-BuLi in hexanes (58 μL, 0.086 mmol, 1.48 M in hexanes) was added dropwise to a cold (−78° C.) solution of phosphine oxide (50 mg, 0.086 mmol) in THF (1.30 mL) under dry argon. The resulting deep red solution was stirred for 1 h, at which time a cold (−78° C.) solution of C,D-ring ketone (Example 3a, 15 mg, 0.043 mmol) in THF (1.2 mL) was added dropwise via cannula. The resulting solution was stirred at −78° C. in the dark for approximately 3 h, then slowly warmed to 40° C. over 2 h. The reaction mixture was quenched with H₂O (1 mL), warmed to rt, extracted with Et₂O (3×10 mL), washed with brine, dried over MgSO₄, filtered, concentrated, and purified by silica gel column chromatography (2050% EtOAc/hexanes) to afford the coupled products as a clear oil. This oil was immediately dissolved in THF (5.0 mL) and treated with TBAF (215 μL, 0.215 mmol, 1.0 M in THF) in the dark for 16 h. Concentration of the reaction mixture and column chromatography (EtOAc) yielded a mixture of diastereomers. This diastereomeric mixture was separated by HPLC (CHIRALCEL® OJ semipreparative column, 15% EtOH/hexanes, 3 mL/min) giving enantiomerically pure, hybrid vitamin-D₃ analogs I(a) (9 mg, 43%, 1α,3β, R_{f}37.2 min) and I(b) (4 mg, 19%, 1β,3α, R_{f}31.7 min). I(a) (1α,3β): [α]_{D}²⁵+31.8 (c 8.3, CHCl₃); ¹H NMR (400 MHz, CDCl₃) δ 7.95-7.88 (m, 2H), 7.70-7.62 (tt, J=7.6, 1.7 Hz, 1H), 7.62-7.53 (m, 2H), 6.35 (d, J=11.2 Hz, 1H), 5.99 (d, J=11.2 Hz, 1H), 5.32 (m, 1H), 4.98 (m, 1H), 4.47-4.38 (m, 1H), 4.27-4.17 (m, 1H), 3.18-3.06 (m, 1H), 3.06-2.92 (m, 1H), 2.86-2.75 (dd, J=12.6, 4.2 Hz, 1H), 2.64-2.53 (dd, J=13.6, 3.2 Hz, 1H), 2.36-2.25 (dd, J=13.4, 6.6 Hz, 1H), 0.88 (d, J=6.0 Hz, 3H), 0.49 (s, 3H); ¹³C NMR (100 MHz, CDCl₃) δ 147.6, 142.5, 139.2, 133.6, 133.2, 129.2, 128.0, 124.8, 117.2, 111.8, 70.8, 66.8, 56.1, 55.7, 53.6, 45.8, 45.2, 42.8, 40.3, 35.0, 28.9, 28.2, 27.3, 23.4, 22.1, 18.5, 12.0; IR (neat) 3647-3119, 3020, 2943, 2871, 1446, 1304, 1216, 1143, 1086, 1055, 753, 688, 534 cm⁻¹, HRMS: calcd for C₂₉H₄₀O₄+Na, 507.2545, found 507.2507; UV pending. I(b) (1β,3α): [α]_{D}²⁵+11.4 (c 2.7, CHCl₃); ¹H NMR (400 MHz, CDCl₃) δ 7.95-7.87 (m, 2H), 7.70-7.62 (tt, J=7.6, 1.7 Hz, 1H), 7.61-7.53 (m, 2H), 6.37 (d, J=11.2 Hz, 1H), 5.99 (d, J=11.2 Hz, 1H), 5.31 (m, 1H), 4.98 (m, 1H), 4.47-4.38 (m, 1H), 4.27-4.16 (m, 1H), 3.18-3.06 (m, 1H), 3.06-2.92 (m, 1H), 2.86-2.75 (dd, J=12.6, 4.2 Hz, 1H), 2.65-2.54 (dd, J=13.6, 3.2 Hz, 1H), 2.35-2.24 (dd, J=13.4, 6.6 Hz, 1H), 0.88 (d, J=6.4 Hz, 3H), 0.50 (s, 3H); ¹³C NMR (100 MHz, CDCl₃) δ 147.2, 142.6, 139.2, 133.6, 133.0, 129.2, 128.0, 124.8, 117.2, 112.5, 71.3, 66.8, 56.1, 55.7, 53.6, 45.8, 45.4, 42.8, 40.3, 35.0, 28.9, 28.2, 27.3, 23.4, 22.1, 18.5, 12.0; IR (neat) 3636-3125, 3066, 3019, 2936, 2866, 1447, 1379, 1306, 1215, 1144, 1085, 1053, 956, 917, 800, 753, 689, 667, 601, 534 cm⁻¹; HRMS: calcd for C₂₉H₄₀O₄+Na, 507.2545, found 507.2533.

In a like manner, the following additional compounds were prepared:

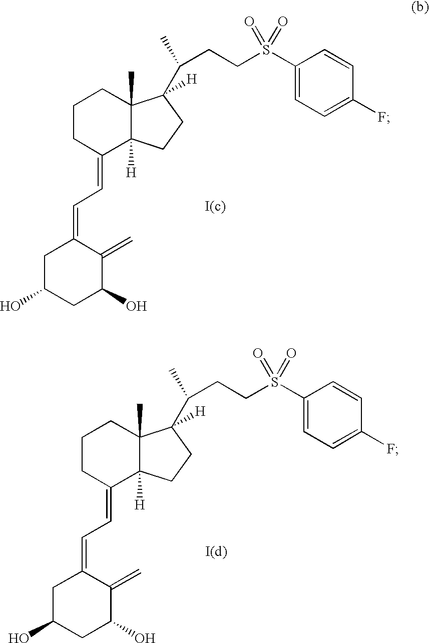

by replacing the compound from Example 4a with the compound of Example 4b. The diastereomers were purified by HPLC (Chiralcel OJ column, 25% EtOH in Hexanes, 2.5 mL/min, 254 nm) to afford 14 mg (55%) of (+)-I(c) (1α,3β, t_{R} 34.2 min) as a viscous oil and 5.3 mg (21%) of (+)-I(d) (1β,3α, t_{R} 27.0 min) as a viscous oil. (+)-I(c): R_{f}0.61 (EtOAc); [α]_{D}²⁶+32.3 (c 1.68, CHCl₃); ¹H NMR (400 MHz, CDCl₃) δ 7.91-7.94 (m, 2H), 7.23-7.27 (m, 2H), 6.36 (d, J=11.2 Hz, 1H), 6.00 (d, J=11.2 Hz, 1H), 5.32 (m, 1H), 4.98 (m, 1H), 4.43 (m, 1H), 4.23 (m, 1H), 3.13 (ddd, J=4.8, 11.6, 14.0 Hz, 1H), 2.99 (ddd, J=4.8, 11.2, 14.0 Hz, 1H), 2.81 (dd, J=4.012.4 Hz, 1H), 2.59 (dd, J=2.8, 13.2 Hz, 1H), 2.31 (dd, J=6.4, 13.2 Hz, 1H), 1.89-2.04 (m, 4H), 1.74-1.87 (m, 2H), 1.62-1.73 (m, 4H), 1.44-1.57 (m, 5H), 1.15-1.30 (m, 3H), 0.89 (d, J=6.4 Hz, 3H), 0.51 (s, 3H); ¹³C NMR (100 MHz, CDCl₃) δ 165.8 (d, 255.1 Hz), 147.6, 142.4, 135.2 (d, 3.0 Hz), 133.2, 130.9 (d, 9.1 Hz), 124.8, 117.3, 116.6 (d, 22.0 Hz), 111.8, 70.8, 66.8, 56.2, 55.7, 53.8, 45.8, 45.2, 42.8, 40.3, 35.0, 28.9, 28.3, 27.3, 23.4, 22.1, 18.5, 12.0; IR (thin film) 3380, 2946, 2874, 1591, 1494, 1315, 1288, 1231, 1143, 1086, 1054, 840, 754, 668 cm⁻¹; HRMS [M+Na] calc'd 525.2445 for C₂₉H₃₉FO₄SNa. found: 525.2462; UV (MeOH) λₘₐₓ 264 nm (ε 14000). (+)-I(d): R_{f}0.61 (EtOAc); [α]_{D}²⁶+21.5 (c 0.57, CHCl₃); ¹H NMR (400 MHz, CDCl₃) δ 7.90-7.95 (m, 2H), 7.25-7.28 (m, 2H). 6.37 (d, J=11.6 Hz, 1H), 6.00 (d, J=11.2 Hz, 1H), 5.31 (dd, J=1.2, 2.0 Hz, 1H), 4.99 (d, J=1.2 Hz, 1H), 4.44 (m, 1H), 4.22 (m, 1H), 3.13 (ddd, J=4.4, 12.0, 14.0 Hz, 1H), 2.99 (ddd, J=4.8, 11.6, 14.0 Hz, 1H), 2.82 (dd, J=4.412.8 Hz, 1H), 2.61 (dd, J=4.0, 13.6 Hz, 1H), 2.29 (dd, J=7.6, 13.2 Hz, 1H), 1.90-2.03 (m, 4H), 1.74-1.87 (m, 2H), 1.44-1.73 (m, 9H), 1.15-1.30 (m, 3H), 0.89 (d, J=6.4 Hz, 3H), 0.51 (s, 3H); ¹³C NMR (100 MHz, CDCl₃) δ 165.8 (d, 255.9 Hz), 147.3, 142.5, 135.3 (d, 3.0 Hz), 133.1, 130.9 (d, 9.9 Hz), 124.8, 117.3, 116.6 (d, 22.8 Hz), 112.5, 71.3, 66.8, 56.1, 55.7, 53.8, 45.8, 45.4, 42.8, 40.3, 35.0, 28.9, 28.3, 27.3, 23.4, 22.2, 18.5, 12.0; IR (thin film) 3382, 2929, 2873, 1591, 1494, 1315, 1288, 1232, 1143, 1086, 1053, 840, 754, 569 cm⁻¹; HRMS [M+Na] calc'd 525.2445 for C₂₉H₃₉FO₄SNa. found: 525.2474; UV (MeOH) lₘₐₓ 258 nm (e 12000).

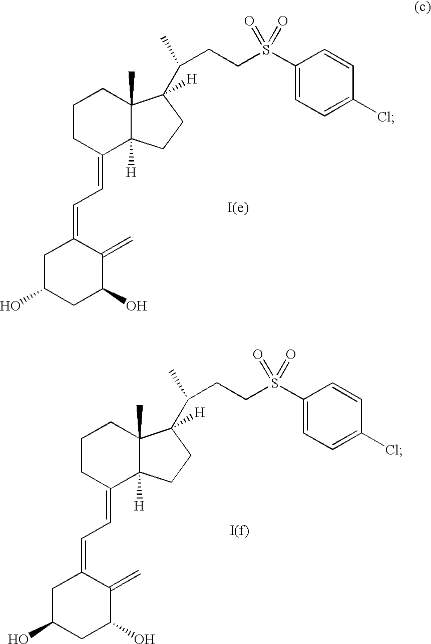

by replacing the compound of Example 4a with the compound of Example 4c; The diastereomers were purified by HPLC (Chiralcel OJ column, 20% EtOH in Hexanes, 2.5 mL/min, 254 nm) to afford 12.3 mg (44%) of (+)-I(e) (1α,3β, t_{R} 29.6 min) as a viscous oil and 3.9 mg (14%) of (+)-I(f) (1β,3α, t_{R} 25.8 min) as a viscous oil. (+)-I(e): R_{f}0.58 (EtOAc); [α]_{D}²⁶+33.5 (c 0.88, CHCl₃); ¹H NMR (400 MHz, CDCl₃) δ 7.83-7.86 (m, 2H), 7.54-7.57 (m, 2H), 6.36 (d, J=11.2, 1H), 6.00 (d, J=11.2 Hz, 1H), 5.33 (s, 1H), 4.99 (s, 1H), 4.43 (m, 1H), 4.23 (m, 1H), 3.13 (ddd, J=4.4, 12.0, 14.0 Hz, 1H), 2.99 (ddd, J=4.8, 11.6, 14.0 Hz, 1H), 2.82 (dd, J=4.0 12.4, 1H), 2.59 (dd, J=3.6, 13.6 Hz, 1H), 2.31 (dd, J=6.8, 13.6 Hz, 1H), 1.44-2.05 (m, 15H), 1.16-1.30 (m, 3H), 0.89 (d, J=6.4 Hz, 3H), 0.51 (s, 3H); ¹³C NMR (100 MHz, CDCl₃) δ 147.6, 142.5, 140.4, 137.7, 133.2, 129.6, 129.5, 124.8, 117.3, 111.8, 70.8, 66.8, 56.2, 55.7, 53.7, 45.8, 45.2, 42.8, 40.3, 35.0, 28.9, 28.3, 27.4, 23.4, 22.2, 18.5, 12.0; IR (thin film) 3382, 2926, 1583, 1313, 1148, 1088, 756 cm⁻¹; HRMS [M+Na] calc'd 541.2150 for C₂₉H₃₉ClO₄SNa. found: 541.2139; UV (MeOH) λₘₐₓ 264 nm (ε 14000). (+)-I(f): R_{f}0.58 (EtOAc); [α]_{D}²⁶+18.4 (c 0.42, CHCl₃); ¹H NMR (400 MHz, CDCl₃) δ 7.83-7.86 (m, 2H), 7.54-7.57 (m, 2H), 6.37 (d, J=11.2, 1H), 6.00 (d, J=11.2 Hz, 1H), 5.32 (m, 1H), 4.99 (m, 1H), 4.44 (m, 1H), 4.22 (m, 1H), 3.13 (ddd, J=4.4, 11.6, 14.0 Hz, 1H), 2.99 (ddd, J=4.8, 11.2, 14.0 Hz, 1H), 2.82 (dd, J=4.0, 12.4, 1H), 2.61 (dd, J=4.0, 12.8 Hz, 1H), 2.30 (dd, J=7.2, 13.2 Hz, 1H), 1.90-2.04 (m, 3H), 1.72-1.88 (m, 3H), 1.44-1.67 (m, 9H), 1.20-1.30 (m, 3H), 0.89 (d, J=6.4 Hz, 3H), 0.51 (s, 3H); ¹³C NMR (100 MHz, CDCl₃) δ 147.3, 142.5, 140.4, 137.7, 133.1, 129.6, 129.5, 124.8, 117.3, 112.5, 71.3, 66.8, 56.1, 55.7, 53.7, 45.8, 45.4, 42.8, 40.3, 35.0, 28.9, 28.3, 27.3, 23.4, 22.2, 18.5, 12.0; IR (thin film) 3366, 2926, 1583, 1475, 1314, 1148, 1089, 758, 668, 630 cm⁻¹; HRMS [M+Na] calc'd 541.2150 for C₂₉H₃₉ClO₄SNa. found: 541.2112; UV (MeOH) λₘₐₓ 253 nm (ε 8700).

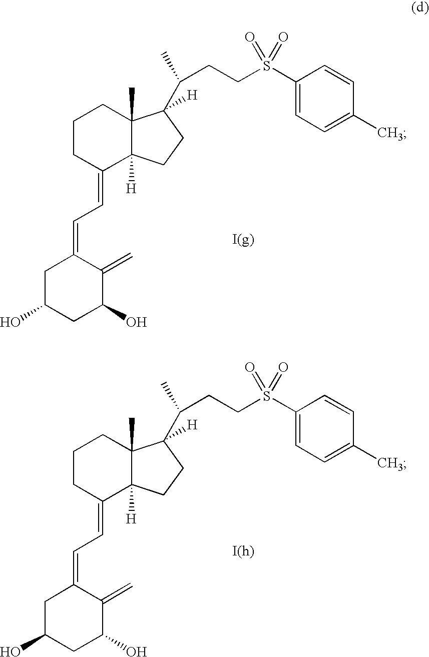

by replacing the compound of Example 4a with the compound of Example 4d.

The diastereomers were purified by HPLC (Chiralcel OJ column, 17% EtOH in Hexanes, 2.5 mL/min, 254 nm) to afford 6.2 mg (53%) of (+)-I(g) (1α,3β, t_{R} 37.7 min) as a viscous oil and 2.0 mg (17%) of (+)-I(h) (1β,3α, t_{R} 31.4 min) as a viscous oil. (+)-I(g): R_{f}0.61 (EtOAc); [α]_{D}²⁶+38.6 (c 0.70, CHCl₃); ¹H NMR (400 MHz, CDCl₃) δ 7.77-7.80 (m, 2H), 7.35-7.37 (m, 2H), 6.36 (d, J=11.2 Hz, 1H), 6.00 (d, J=11.6 Hz, 1H), 5.32 (dd, J=1.6, 1.6 Hz, 1H), 4.99 (dd, J=1.2, 1.2 Hz, 1H), 4.43 (m, 1H), 4.23 (m, 1H), 3.11 (ddd, J=4.8, 12.0, 14.0 Hz, 1H), 2.97 (ddd, J=4.8, 11.2, 13.6 Hz, 1H), 2.81 (dd, J=4.4, 12.4 Hz, 1H), 2.59 (dd, J=4.0, 13.2 Hz, 1H), 2.46 (s, 3H), 2.31 (dd, J=6.4, 13.2 Hz, 1H), 1.89-2.03 (m, 4H), 1.74-1.86 (m, 2H), 1.43-1.72 (m, 8H), 1.17-1.30 (m, 4H), 0.88 (d, J=6.0 Hz, 3H), 0.50 (s, 3H); ¹³C NMR (100 MHz, CDCl₃) δ 147.6, 144.5, 142.6, 136.3, 133.2, 129.8, 128.0, 124.8, 117.2, 111.8, 70.8, 66.8, 56.2, 55.7, 53.7, 45.8, 45.2, 42.8, 40.3, 35.0, 28.9, 28.3, 27.3, 23.4, 22.1, 21.6, 18.5, 12.0; IR (thin film) 3392, 2926, 2873, 1597, 1448, 1313, 1302, 1285, 1142, 1087, 1054, 754, 668 cm⁻¹; HRMS [M+Na] calc'd 521.2696 for C₃₀H₄₂O₄SNa. found: 521.2662; UV (MeOH) λₘₐₓ 262 nm (ε 18000). (+)-I(h): R_{f}0.61 (EtOAc); [α]_{D}²⁶+22.8 (c 0.20, CHCl₃); ¹H NMR (400 MHz, CDCl₃) δ 7.77-7.79 (m, 2H), 7.35-7.37 (m, 2H), 6.37 (d, J=11.6 Hz, 1H), 5.99 (d, J=10.8 Hz, 1H), 5.31 (dd, J=1.2, 1.2 Hz, 1H), 4.99 (m, 1H), 4.44 (m, 1H), 4.22 (m, 1H), 3.11 (ddd, J=4.0, 12.0, 14.0 Hz, 1H), 2.97 (ddd, J=4.8, 11.2, 14.0 Hz, 1H), 2.81 (dd, J=4.8, 12.8 Hz, 1H), 2.61 (dd, J=4.0, 13.6 Hz, 1H), 2.46 (s, 3H), 2.29 (dd, J=7.6, 13.2 Hz, 1H), 1.90-2.04 (m, 3H), 1.74-1.87 (m, 2H), 1.43-1.70 (m, 9H), 1.17-1.29 (m, 4H), 0.88 (d, J=6.4 Hz, 3H), 0.50 (s, 3H); ¹³C NMR (100 MHz, CDCl₃) δ 147.3, 144.5, 142.6, 136.3, 133.0, 129.8, 128.0, 124.8, 117.2, 112.5, 71.3, 66.8, 56.2, 55.7, 53.7, 45.8, 45.4, 42.8, 40.3, 35.0, 28.9, 28.4, 27.3, 23.4, 22.2, 21.6, 18.5, 12.0; IR (thin film) 3400, 2926, 2872, 1597, 1449, 1313, 1302, 1286, 1142, 1087, 1053, 754, 668 cm⁻¹; HRMS [M+Na] calc'd 521.2696 for C₃₀H₄₂O₄SNa. found: 521.2707; UV (MeOH) λₘₐₓ 264 nm (ε 8700).

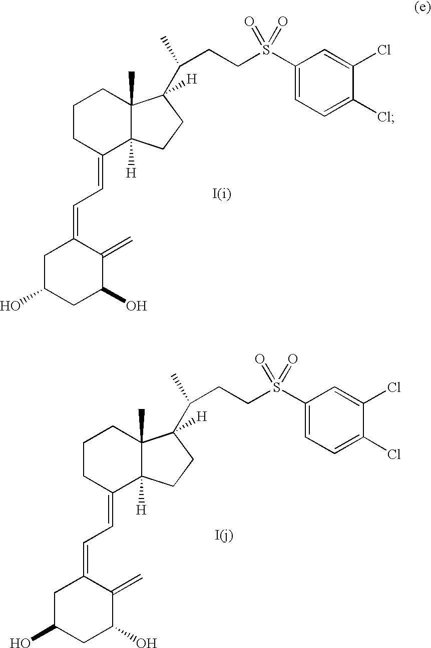

by replacing the compound of Example 4a with the compound of example 4e;

The diastereomers were purified by HPLC (Chiralcel OJ column, 22% EtOH in Hexanes, 2.5 mL/min, 254 nm) to afford 17.7 mg (52%) of (+)-I(i) (1α,3β, t_{R} 36.4 min) as a viscous oil and 5.3 mg (16%) of (+)-I(j) (1β,3α, t_{R} 29.0 min) as a viscous oil. (+)-I(i): R_{f}0.73 (EtOAc); [α]_{D}²⁶+28.3 (c 2.10, CHCl₃); ¹H NMR (400 MHz, CDCl₃) δ 8.00 (d, J=2.0 Hz, 1H), 7.73 (dd, J=2.4, 8.4 Hz, 2H), 7.66 (d, J=8.4 Hz, 1H), 6.36 (d, J=11.6 Hz, 1H), 6.01 (d, J=11.2 Hz, 1H), 5.33 (dd, J=1.2, 1.6 Hz, 1H), 4.99 (m, 1H), 4.43 (dd, J=4.4, 7.6 Hz, 1H), 4.23 (m, 1H), 3.14 (ddd, J=4.4, 12.0, 14.0 Hz, 1H), 3.00 (ddd, J=4.8, 11.2, 14.0 Hz, 1H), 2.82 (dd, J=4.4, 12.0 Hz, 1H), 2.59 (dd, J=3.6, 13.6 Hz, 1H), 2.31 (dd, J=6.4, 13.6 Hz, 1H), 1.92-2.04 (m, 4H), 1.76-1.90 (m, 2H), 1.64-1.71 (m, 4H), 1.45-1.58 (m, 5H), 1.20-1.31 (m, 3H), 0.90 (d, J=6.0 Hz, 3H), 0.52 (s, 3H); ¹³C NMR (100 MHz, CDCl₃) δ 147.6, 142.4, 139.0, 138.8, 134.1, 133.3, 131.4, 130.0, 127.1, 124.8, 117.3, 111.8, 70.8, 66.8, 56.1, 55.7, 53.7, 45.8, 45.2, 42.8, 40.3, 35.0, 28.9, 28.2, 27.4, 23.4, 22.1, 18.4, 12.0; IR (thin film) 3375, 2945, 1454, 1370, 1316, 1149, 1093, 1053, 1034, 824, 754, 676, 633 cm⁻¹; HRMS [M+Na] calc'd 575.1760 for C₂₉H₃₈Cl₂O₄SNa. found: 575.1764; UV (MeOH) λₘₐₓ 262 nm (ε 12000). (+)-I(j): R_{f}0.73 (EtOAc); [α]_{D}²⁶+22.6 (c 0.54, CHCl₃); ¹H NMR (400 MHz, CDCl₃) δ 8.00 (d, J=2.0 Hz, 1H), 7.73 (dd, J=2.0, 8.4 Hz, 2H), 7.66 (d, J=8.4 Hz, 1H), 6.37 (d, J=11.2 Hz, 1H), 6.00 (d, J=11.2 Hz, 1H), 5.32 (m, 1H), 4.99 (m, 1H), 4.44 (m, 1H), 4.22 (m, 1H), 3.14 (ddd, J=4.4, 12.0, 14.0 Hz, 1H), 3.00 (ddd, J=4.4, 11.6, 14.0 Hz, 1H), 2.82 (dd, J=4.4, 12.4 Hz, 1H), 2.61 (dd, J=4.0, 13.2 Hz, 1H), 2.30 (dd, J=7.6, 13.2 Hz, 1H), 1.90-2.04 (m, 4H), 1.76-1.89 (m, 2H), 1.46-1.72 (m, 9H), 1.20-1.31 (m, 3H), 0.91 (d, J=6.0 Hz, 3H), 0.52 (s, 3H); ¹³C NMR (100 MHz, CDCl₃) δ 147.2, 142.4, 139.0, 138.8, 134.1, 133.1, 131.4, 130.1, 127.1, 124.8, 117.3, 112.6, 71.3, 66.8, 56.1, 55.7, 53.8, 45.8, 45.4, 42.8, 40.3, 35.0, 28.9, 28.2, 27.4, 23.4, 22.2, 18.5, 12.0; IR (thin film) 3371, 2945, 2872, 1454, 1370, 1316, 1149, 1093, 1053, 1034, 824, 754, 676 cm⁻¹; HRMS [M+Na] calc'd 575.1760 for C₂₉H₃₈Cl₂₀₄SNa. found: 575.1764; UV (MeOH) λₘₐₓ 264 nm (ε 11000).

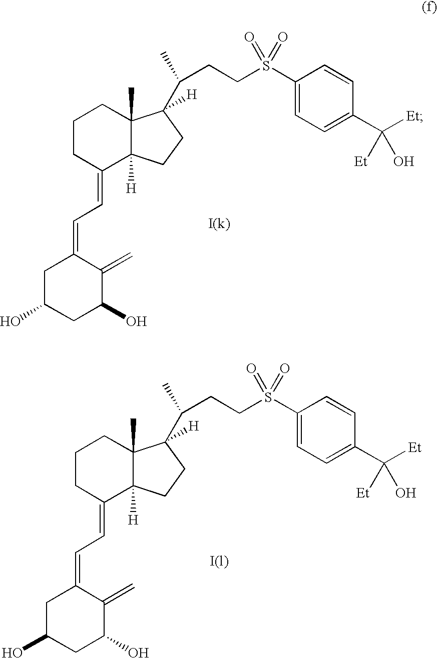

by replacing the compound of Example 4a with the compound of example 4f.

The diastereomers were then purified by HPLC (Chiralcel OJ column, 15% EtOH in Hexanes, 2.5 mL/min, 254 nm) to afford 8.2 mg (47%) of (+)-I(k) (1α,3β, t_{R} 36.3 min) as a viscous oil and 5.1 mg (29%) of I(l)) (1β,3α, t_{R} 30.5 min) as a viscous oil. (+)-I(k): R_{f}0.56 (EtOAc); [α]_{D}²⁶+25.1 (c 2.10, CHCl₃); ¹H NMR (400 MHz, CDCl₃) δ 7.85-7.88 (m, 2H), 7.58-7.60 (m, 2H), 6.36 (d, J=10.8 Hz, 1H), 5.99 (d, J=11.2 Hz, 1H), 5.32 (dd, J=1.2, 2.0 Hz, 1H), 4.98 (m, 1H), 4.43 (dd, J=4.4, 7.6 Hz, 1H), 4.22 (tt, J=3.6, 6.4 Hz, 1H), 3.13 (ddd, J=4.4, 12.0, 14.0 Hz, 1H), 3.00 (ddd, J=4.8, 11.2, 13.6 Hz, 1H), 2.81 (dd, J=4.4, 12.4 Hz, 1H), 2.59 (dd, J=3.2, 13.6 Hz, 1H), 2.31 (dd, J=6.4, 13.6 Hz, 1H), 1.79-2.05 (m, 10H), 1.40-1.75 (m, 9H), 1.11-1.29 (m, 4H), 0.89 (d, J=6.4 Hz, 3H), 0.75 (t, J=7.2 Hz, 6H), 0.49 (s, 3H); ¹³C NMR (100 MHz, CDCl₃) δ 152.2, 147.6, 142.5, 137.0, 133.2, 127.8, 126.5, 124.8, 117.2, 111.8, 77.4, 70.8, 66.8, 56.2, 55.6, 53.6, 45.8, 45.2, 42.8, 40.3, 35.29, 35.26, 35.0, 29.0, 28.3, 27.2, 23.4, 22.1, 18.5, 12.0, 7.6; IR (thin film) 3456, 2937, 1458, 1311, 1144, 1086, 1054, 967, 755 cm⁻¹; HRMS [M+Na] calc'd 593.3271 for C₃₄H₅₀O₅SNa. found: 593.3237; UV (MeOH) λₘₐₓ 261 nm (ε 8600). I(l): R_{f}0.73 (EtOAc); ¹H NMR (400 MHz, CDCl₃) δ 7.86-7.88 (m, 2H), 7.58-7.61 (m, 2H), 6.37 (d, J=11.2 Hz, 1H), 5.98 (d, J=11.2 Hz, 1H), 5.31 (m, 1H), 4.98 (m, 1H), 4.43 (dd, J=4.4, 6.4 Hz, 1H), 4.21 (tt, J=3.6, 7.2 Hz, 1H), 3.13 (ddd, J=4.4, 12.0, 14.0 Hz, 1H), 3.00 (ddd, J=4.8, 11.2, 14.0 Hz, 1H), 2.81 (dd, J=4.4, 12.8 Hz, 1H), 2.61 (dd, J=4.0, 13.2 Hz, 1H), 2.29 (dd, J=7.2, 13.2 Hz, 1H), 1.80-2.03 (m, 10H), 1.40-1.75 (m, 9H), 1.13-1.29 (m, 4H), 0.89 (d, J=6.4 Hz, 3H), 0.75 (t, J=7.2 Hz, 6H), 0.49 (s, 3H); The title compound I(l) was decomposed during overnight ¹³C NMR.

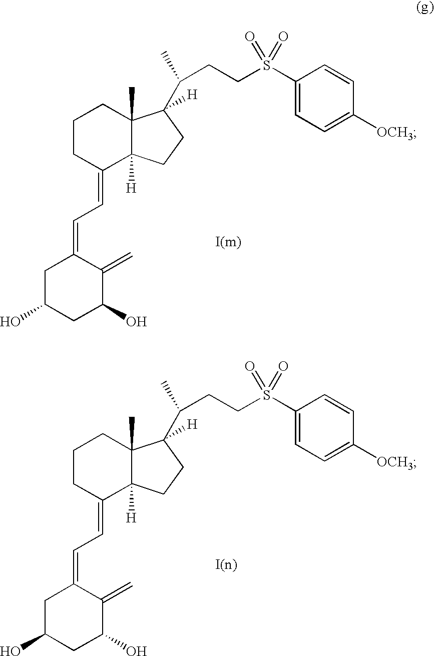

by replacing the compound of Example 4a with the compound of example 4h;

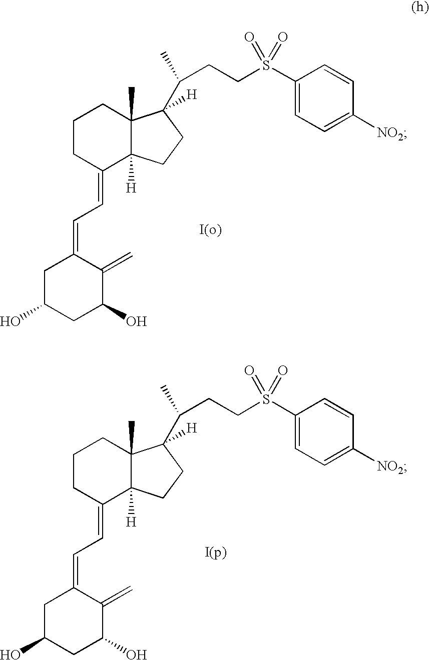

by replacing the compound of Example 4a with the compound of example 4i; and

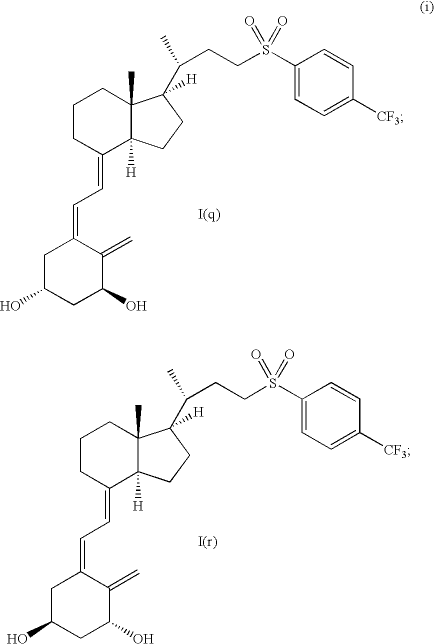

by replacing the compound of Example 3a with the compound of example 4j.

### Example 6

### Prepararion of 24-SO₂—PhCH(NOMe) I(s) and I(t)

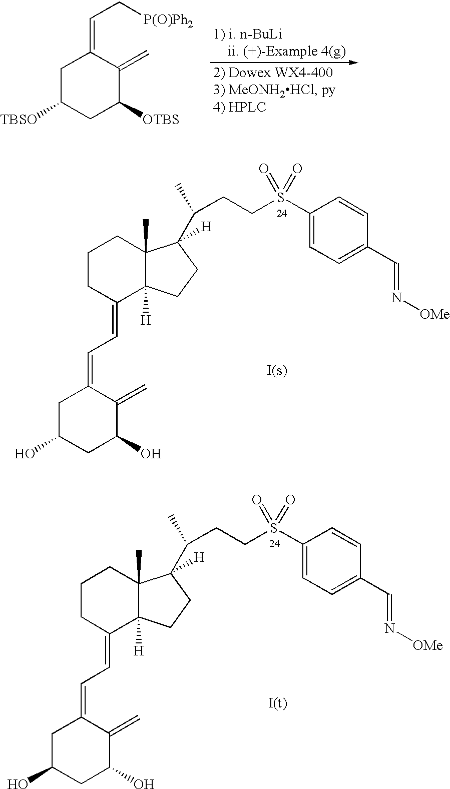

To a solution of (±)-A-ring phosphine oxide (Posner, G. H. et al. J. Med. Chem. 1992, 35, 3280-3287) (72.8 mg, 0.125 mmol) in THF (2 mL) was added 0.047 mL of n-BuLi (2.67 M in Hexane, 0.125 mmol) at −78° C., then the reddish solution was stirred for 10 min at the same temperature. A precooled (−78° C.) solution of C24-p-acetalphenyl sulfone C/D ring ketone from Example 4g (32.4 mg, 0.0770 mmol) in THF (2 mL) was added to the above solution at −78° C. via cannula. The resulting reddish orange solution was stirred for 6 hrs at −78° C. The reaction was quenched with 2 mL of pH 7 buffer, then warmed to room temperature, extracted with EtOAc, washed with brine, dried over MgSO₄, filtered, concentrated in vacuo, and purified by flash chromatography (EtOAc:Hex=1:4) to give 31.5 mg (52%) of a diastereomeric mixture of bis TBS protected p-acetalphenyl sulfone coupled products. A solution of this latter product (20 mg, 0.0255 mmol) and Dowex 50WX4-400 (794 mg) in CH₂Cl₂-acetone (2 mL-2 mL) was stirred for 24 h. The reaction mixture was filtered and purified by flash chromatography (EtOAc:Hex=1:1 to 2:1) to afford 5.4 mg of a diastereomeric mixture of the corresponding bishydroxy benzaldehydes along with some unreacted starting material. The latter mixture was treated with MeONH₂.HCl (8.9 mg, 0.104 mmol), several beads of molecular sieves 4A, and pyridine (1.2 mL). The reaction mixture was diluted with EtOAc, washed with 1N aq. HCl and brine, dried over MgSO₄, filtered, and concentrated in vacuo to give a crude mixture which was then purified by flash chromatography (EtOAc:Hex=3:1) to afford 3.7 mg (65%) of a diastereomeric mixture of I(s) and I(t). The diastereomers were then purified by HPLC (Chiralcel OJ column, 30% EtOH in Hexanes, 2.5 mL/min, 254 nm) to afford I(s) (1α,3β, t_{R} 32.7 min) as a viscous oil and I(t) (1β,3α, t_{R} 25.1 min) as a viscous oil.

### Example 7

### Preparation of Compound I(u)

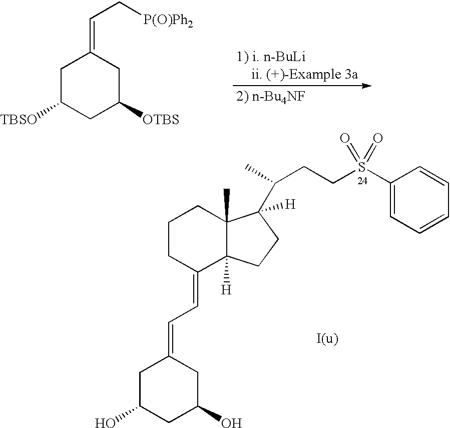

A solution of 58 mg (0.10 mmol) of 19-nor-phosphine oxide (Hilpert, H. and Wirz, B. Tetrahedron 2001, 57, 681-694) in 2.0 mL of anhydrous THF was cooled to −78° C. and treated with 64 μL (0.10 mmol, 1.6 M in hexanes) of n-BuLi under argon atmosphere. The mixture turned deep reddish and was stirred for 15 min at −78° C. To the solution was added dropwise a precooled (−78° C.) solution of 12 mg (0.034 mmol) of the C,D-ring ketone from Example 3a in 1.5 mL of anhydrous THF via cannula. The reaction kept going until the reddish orange color faded to yellow (about 4 hr). The reaction was quenched by adding 1.0 mL of pH 7 buffer at −78° C., then warmed to room temperature, extracted with EtOAc (20 mL×2), washed with brine, dried over MgSO₄, concentrated. The residue was subjected to column chromatography with EtOAc/hexanes (1/3) as eluent to afford 19 mg (80%) of the coupled product as a colorless oil.

The coupled product (19 mg, 0.027 mmol) was dissolved in 3 mL of anhydrous THF, and to the solution was added 0.40 mL (0.40 mmol) of a 1.0 M solution of TBAF in THF. The resulting mixture was stirred overnight at room temperature, then quenched with 2 mL of water. The solution was extracted with EtOAc (20 mL×3), washed with brine, dried over MgSO₄, concentrated. The residue was subjected to column chromatography with EtOAc as eluent to give 12 mg (94%) of the crude product of (+)-I(u) as a colorless oil. The crude product was purified by HPLC (Chiralcel OJ column, 20% EtOH in Hexanes, 2.5 mL/min, 254 nm) to afford 10.5 mg of (+)-I(u) (1a,3b, t_{R}=29.1 min).: [α]²⁴_{D}=+91.2 (c=0.19, MeOH). ¹H NMR (400 MHz, CDCl₃) δ 7.90-7.93 (m, 2H), 7.67 (m, 1H), 7.56-7.60 (m, 2H), 6.29 (d, J=11.2 Hz, 1H), 5.83 (d, J=11.2 Hz, 1H), 4.11 (m, 1H), 4.05 (m, 1H), 3.14 (ddd, J=13.6, 12.0, 4.0 Hz, 1H), 3.00 (ddd, J=13.6, 11.2, 4.8 Hz, 1H), 2.78 (dd, J=12.4, 4.0 Hz, 1H), 2.72 (dd, J=13.2, 4.0 Hz, 1H), 2.47 (dd, J=13.2, 3.6 Hz, 1H), 2.17-2.43 (m, 2H), 1.74-1.99 (m, 6H), 1.44-1.68 (m, 9H), 1.17-1.30 (m, 3H), 0.89 (d, J=6.0 Hz, 3H), 0.50 (s, 3H). ¹³C NMR (100 MHz, CDCl₃) δ 142.4, 139.2, 133.6, 131.4, 129.2, 128.0, 123.7, 115.5, 67.4, 67.2, 56.1, 55.7, 53.6, 45.7, 44.6, 42.1, 40.3, 37.1, 35.0, 28.8, 28.3, 27.3, 23.3, 22.1, 18.5, 12.0. IR (neat, cm⁻) 3362, 2943, 1447, 1306, 1145, 1086, 1048, 753, 689, 537. HRMS ([M+Na]⁺) calcd. 495.2539, found 495.2526.

### Example 8

### Preparation of Aldehyde (+)-XI

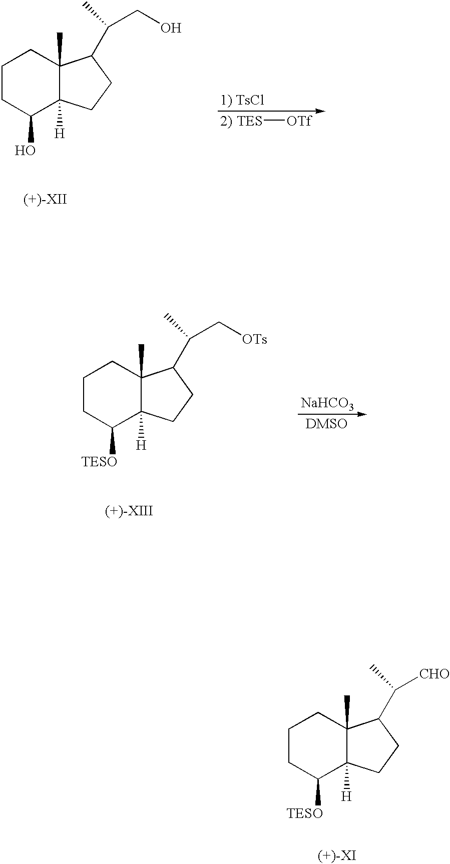

(a) Preparation of Lythgoe diol (+)-XII: As described in Posner G. H. et al. J. Org. Chem. 1997, 62, 3299-3314.

(b) Preparation of TES Tosylate (+)-XIII: To a solution of the diol (+)-XII (364 mg, 1.64 mmol eq) and DMAP (341 mg, 1.7 eq) in 15 mL of CH₂Cl₂ was slowly added the soluion of p-toluenesulfonyl chloride (360 mg, 1.2 eq) in 5 mL of CH₂Cl₂ at 0° C. After being stirred for 16 h at 0° C., the reaction mixture was cooled to −78° C. To this was added 2,6-lutidine (0.95 mL) and TESOTf (1.1 mL) successively with monitoring by TLC. Upon the completion of reaction, the mixture was diluted with ether, successively washed with diluted HCl to remove 2,6-lutidine followed by brine. The organic extract was dried over MgSO₄, concentrated in vacuo, and then purified by chromatography (25% EtOAc/hexanes) to give 708 mg (90%) of the desired TES tosylate (+)-XIII as a colorless oil. [α]²⁵_{D}−12° (c 2.3, EtOAc); ¹H NMR (CDCl₃) δ 7.79 (d, J=8.0 Hz, 2H), 7.33 (d, J=8.0 Hz, 2H), 4.17 (m, 2H), 3.84 (dd, J=8.0, 4.8 Hz, 1H), 2.43 (s, 3H), 1.80 (m, 2H), 1.52 (m, 4H), 1.33 (s, 3H), 1.22 (s, 3H), 0.87 (t, J=7.6 Hz, 3H), 0.83 (t, J=7.6 Hz, 3H); ¹³C NMR (CDCl₃) δ 144.69, 132.94, 129.78, 127.82, 106.81, 83.80, 76.72, 68.33, 29.39, 28.36, 27.15, 26.82, 25.36, 21.6, 8.19, 7.23; IR (CDCl₃, cm⁻¹) 2941, 2860, 1732, 1592, 1458, 1354; HRMS (Cl) m/z (M+H+) calcd. 357.1736 for C₁₈H₂₈O₅S, found 357.1741.

(c) Preparation of Aldehyde (+)-XI: According to the method of Komblum, et al. J. Am. Chem. Soc. 1959, 81, 4113-3116, to a solution of primary tosylate (+)-XIII (708 mg, 0.147 mmol) in DMSO (10 mL) was added NaHCO₃ (495 mg, 5.9 mmol) and heated to 150° C. When the evolution of gas had ceased (10-15 min) the reaction mixture was cooled rapidly to rt (water bath), diluted with water (50 mL), and extracted (×2) with ether. The organic fractions were combined, washed repeatedly with brine, dried with Na₂SO₄, and concentrated to a light oil. Purification by flash silica gel chromatography (2% EtOAc/hexanes) provided 120 mg (80%) of aldehyde (+)-XI as a colorless oil: [α]²⁵_{D}+40.7° (c 2.3, CHCl₃); ¹H NMR (400 MHz, CDCl₃) δ 9.54 (d, J=3.2 Hz, 1H), 4.03 (m, 1H), 2.32 (ddq, J=10.0, 6.8, 3.2 Hz, 1H), 1.73-1.92 (m, 3H), 1.58-1.71 (m, 2H), 1.28-1.44 (m, 5H), 1.10-1.26 (m, 2H), 1.06 (d, J=6.8 Hz, 3H) 0.93 (s, 3H), 0.91 (t, J=8.0 Hz, 9H) 0.52 (q, J=8.0 Hz, 6H); ¹³C NMR (100 MHz, CDCl₃) δ 205.2, 69.0, 52.3, 51.6, 49.1, 42.6, 40.4, 34.5, 26.2, 23.3, 17.6, 13.9, 13.3, 6.9, 4.9; IR (thin film cm⁻¹) 2948, 2872, 1724, 1456, 1164.

### Example 9

### Preparation of Ketones (+)-V (R⁴=t-butyl) and (+)-V (R⁴=isopropyl)

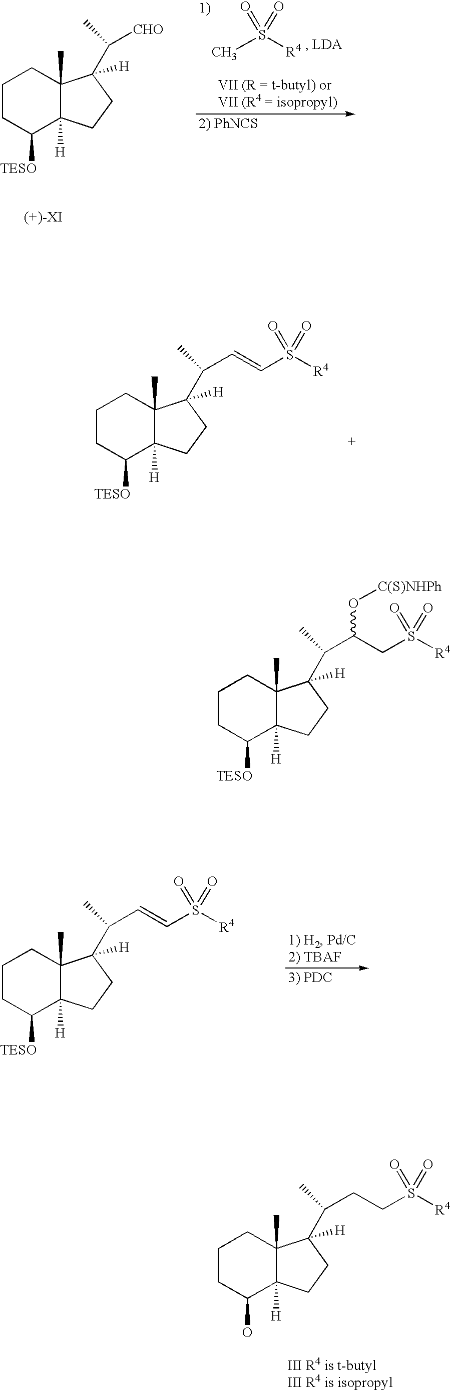

(a) Preparation of tert-Butyl methyl sulfone VII (R⁴=t-butyl) and isopropyl methyl sulfone VII (R⁴=isopropyl): To a solution of tert-butyl methyl sulfide (5.0 g, 0.048 mol) in methanol (125 ml) was added oxone (21.9 g, 0.144 mol) in H₂O (125 ml) at 0° C. The mixture was warmed to ambient temperature and allowed to stir overnight. The mixture was concentrated to constant volume, diluted with water (150 mL), extracted with CH₂Cl₂ (6×50 mL), dried over MgSO₄ and concentrated in vacuo to provide sulfone VII, where R⁴ is t-butyl (6.20 g, 95%) as a white solid. ¹H NMR (400 MHz, CDCl₃) δ 2.82 (s, 3H), 1.44 (s, 9H); ¹³C NMR (100 MHz, CDCl₃) δ 59.62, 35.10, 24.37. Isopropyl methyl sulfone VII, where R⁴ is isopropyl, can be prepared in the same manner by oxidizing isopropyl methyl sulfide, instead of tert-butyl methyl sulfide.

(b) Preparation of α,β-unsaturated sulfone wherein R⁴ is t-butyl: To a solution of diisopropylamine (91 mL, 1.5 eq) in THF (3 mL) was added 1.6 M solution of n-BuLi hexanes (0.4 mL, 1.5 eq) at −78° C., and then it was stirred for an additional 30 min at −78° C. and another 30 min at −35° C. A solution of t-butylmethyl sulfone VII (R⁴=t-butyl) (143 mg, 1.5 eq) in THF (1 mL) was added to the LDA solution at −78° C. After being stirred for 1 h, the solution was treated with a solution of the aldehyde (+)-XI (130 mg, 0.44 mmol) in THF (0.5 mL) by dropwise addition. The reaction mixture was stirred for 15 min at the same temperature, quenched with a solution of phenylisothiocyanate (PhNCS) (0.15 mL, 1.6 eq) in THF (1 mL), and then warmed to rt. After being stirred for 30 min at rt, the reaction mixture was extracted with ether (50 mL×2), washed with saturated NaHCO₃ solution, brine, dried over MgSO₄, concentrated in vacuo, and then purified by chromatography (10% EtOAc/hexanes) to give 95 mg (49%) of the α,β-unsaturated sulfone and 73 mg (31%) of corresponding phenylthianocarbamate as diasteromeric mixtures. [α]²⁵_{D}+56° (c 9.4, CHCl₃); ¹H NMR (400 MHz, CDCl₃) δ 6.73 (dd, J=15.2 Hz, 9.2, 1H), 6.14 (d, J=15.2, 1H), 4.03 (br d, J=2.4, 1H), 1.90-1.94 (dm, J=12.4 Hz, 1H), 1.54-1.84 (m, 4H), 1.34 (s, 9H), 1.12-1.27 (m, 5H), 1.09 (d, J=6.4 Hz, 3H), 0.94 (s, 3H), 0.93 (t, J=8.0 Hz, 9H), 0.54 (q, J=8.0 Hz, 6H); ¹³C NMR (100 MHz, CDCl₃) δ 156.55, 121.30, 69.09, 58.29, 55.24, 52.78, 42.53, 41.90, 40.57, 39.57, 34.45, 27.66, 23.35, 23.03, 18.96, 17, 13.79, 6.91, 4.90; MS m/z (70 e V, Cl) 460 (M+NH₄⁺); HRMS m/z (M⁺) Calcd. 460.3281 for C₂₄H₄₆O₃SSi found 460.3292; IR (neat, cm⁻¹) 2951, 2875, 1631, 1457, 1304.

(c) Preparation of α,β-unsaturated sulfone wherein R⁴ is isopropyl: A solution of aldehyde (+)XI (232 mg, 0.78 mmol) in THF (2 mL) was reacted with the anion of isopropyl methyl sulfone VII (R⁴=isopropyl) (143 mg, 1.5 eq) in THF (3.0 mL) as decribed in part (b) to give 54 mg (18%) of the α,β-unsaturated isopropyl sulfone and 351 mg (81%) of the corresponding phenylthianocarbamate as diasteromeric mixtures. ¹H NMR (400 MHz, CDCl₃) δ 6.75 (dd, J=9.2, 15.2 Hz, 1H), 6.11 (d, J=15.2, 1H), 4.03 (d, J=2.4, 1H), 3.17 (septet, J=6.8 Hz, 1H), 2.33-2.39 (m, 1H), 2.06-2.17 (m, 2H), 1.76-1.81 (m, 1H), 1.53-1.71 (m, 3H), 1.32-1.39 (m, 4H), 1.16-1.27 (m, 3H), 1.33 (d, J=7.2 Hz, 6H), 1.10 (d, J=6.8 Hz, 3H), 0.95 (s, 3H), 0.94 (t, J=8.0 Hz, 9H), 0.55 (q, J=8.0 Hz, 6H).

(d) Preparation of C/D ring ketone (+)-III, wherein R⁴ is t-butyl: A solution of α,β unsaturated sulfone from part (b) (94 mg, 0.21 mmol) in benzene (10 mL) was hydrogenated (50 psi) for 2 days in the presence of 10 mg of 10% Pd/C until the absence of starting material was indicated by TLC. The reaction mixture was filtered through a bed of Celite™ with several benzene washes and the filtrate was concentrated to a light oil. The resulting mixture was treated with TBAF in THF followed by normal aqueous work-up, then purified by chromatography (40% EtOAc/hexanes) to give 70 mg (98%) of alcohol as a white solid: mp. 129-131° C.; [α]²⁵_{D}+37° (c 4.3, CHCl₃); ¹H NMR (400 MHz, CDCl₃) δ ¹H NMR (400 MHz, CDCl₃) δ 4.05 (br d, J=2.4, 1H), 2.92 (td, J=12.8, 4.4 Hz, 1H), 2.72-2.79 (m, 1H), 1.73-2.02 (m, 5H), 1.26-1.61 (m, 8H), 1.7 (d, J=7.2 Hz, 6H), 1.27-1.60 (m, 3H), 1.37 (s, 9H), 1.02-1.18 (m, 2H), 0.91 (d, J=6.8 Hz, 3H), 0.90 (s, 3H); ¹³C NMR (100 MHz, CDCl₃) δ 69.07, 58.89, 56.17, 52.45, 42.96, 41.90, 40.28, 34.72, 33.46, 27.02, 26.00, 23.48, 22.39, 18.23, 17.36, 13.50; MS m/z (70 eV, Cl) 348 (M+NH₄⁺); HRMS m/z (M⁺) Cald. 330.2229 for C₁₈H₃₄O₃S, found 330.2236; IR (CHCl₃, cm⁻¹) 3519, 2942, 2872, 1464, 1299, 1281, 1116. To a solution of the alcohol (71 mg, 0.21 mmol) in CH₂Cl₂ (5 mL), were added 0.24 g of oven dried Celite™ and PDC (0.24 g, 3.0 eq) at rt. After stirring at rt for 16 h, the mixture was passed through 2 cm of flash silica gel pad, washed with EtOAc. The filtrate was concentrated in vacuo, and then chromatographed with 30% EtOAc in hexanes to give 61 mg (86%) of the ketone (+)-III, where R⁴ is t-butyl, as a white solid: mp. 123-125° C.; [α]²⁵_{D}+14° (c 4.9, CHCl₃); ¹H NMR (400 MHz, CDCl₃) δ 2.90-2.98 (m, 1H), 2.74-2.82 (m, 1H), 2.44 (dd, J=11.6, 7.2 Hz, 1H), 2.15-2.28 (m, 2H), 1.80-2.10 (m, 4H), 1.66-1.77 (m, 1H), 1.35-1.64 (m, 7H), 1.39 (s, 9H), 0.97 (d, J=6.4 Hz, 3H), 0.62 (s, 3H); ¹³C NMR (100 MHz, CDCl₃) δ 211.47, 61.67, 58.91, 49.75, 42.83, 40.78, 38.78, 34.91, 27.33, 26.09, 23.88, 23.42, 18.96, 18.37, 12.45; MS m/z (70 eV, Cl) 346 (M+NH₄); HRMS m/z (M⁺) Calcd. 328.2072 for C₁₈H₃₂O₃S, found 328.2076; IR (CHCl₃, cm⁻¹) 3020, 2964, 2877, 1707, 1464, 1298, 1280, 1116.

(e) Preparation of C/D ring ketone (+)-III, wherein R⁴ is isopropyl: A solution of α,β unsaturated sulfone from part (c) (54 mg, 0.13 mmol) in benzene (5 mL) was hydrogenated (50 psi) for 2 days in the presence of 10 mg of 10% Pd/C until the absence of starting material was indicated by TLC. The reaction mixture was filtered through a bed of Celite™ with several benzene washes and the filtrate was concentrated to a light oil. The resulting mixture was treated with TBAF in THF followed by normal aqueous work-up, then purified by chromatography (40% EtOAc/hexanes) to give 33 mg (78%) of alcohol as a colorless oil. [α]²⁵_{D}+37° (c 3.3, CHCl₃); ¹H NMR (400 MHz, CDCl₃) δ 4.05 (br, d J=2.4, 1H), 3.17 (septet, J=6.8 Hz, 1H) 2.92-3.00 (m, 1H), 2.76-2.83 (m, 1H), 1.73-1.97 (m, 5H), 1.26-1.61 (m, 8H), 1.37 (d, J=6.8 Hz, 6H), 1.03-1.17 (m, 2H), 0.93 (d, J=6.0 Hz, 3H) 0.92 (s, 3H); ¹³C NMR (100 MHz, CDCl₃) δ 69.04, 55.93, 52.49, 52.46, 46.56, 41.90, 40.28, 34.56, 33.47, 27.06, 26.93, 22.40, 18.24, 17.36, 15.36, 15.20, 13.51; MS m/z (70 eV, CT) 348 (M+NH₄⁺); HRMS m/z (M⁺) Calcd. 330.2229 for C₁₈H₃₄O₃S, found 330.2236; IR (neat cm⁻¹) 3519, 2942, 2872, 1464, 1299, 1281, 1116. The alcohol was oxidized with PDC in the same manner as described in part (d) to give 28 mg (86%) of the desired ketone III, where R⁴ is isopropyl, as a colorless oil. [α]²⁵_{D}+17° (c 2.8, CHCl₃); ¹H NMR (400 MHz, CDCl₃) δ; 3.08 (septet, J=6.8 Hz, 1H), 2.93-3.00 (m, 1H), 2.78-2.85 (m, 1H), 2.45 (dd, J=11.6, 7.6 Hz, 1H), 2.16-2.30 (m, 2H), 1.68-2.10 (m, 6H), 1.52-1.63 (m, 5H), 1.37 (d, J=6.8 Hz, 6H), 1.32-1.45 (m, 2H), 0.99 (d, J=6.0 Hz, 3H), 0.63 (s, 3H); ¹³C NMR (100 MHz, CDCl₃) δ 210.40, 61.70, 56.00, 52.66, 49.75, 46.44, 40.81, 38.81, 34.79, 27.39, 26.94, 28.90, 18.99, 18.40, 15.34, 15.21, 12.48; MS m/z (70 eV, Cl) 348 (M+NH₄⁺); HRMS m/z (M⁺) Calcd. 330.2229 for C₁₈H₃₄O₃S, found 330.2236; IR (neat, cm⁻¹) 2957, 2877, 1710, 1467, 1306, 1262, 1130.

### Example 10

### Preparation of Compounds of Formula I(v) and I(w)

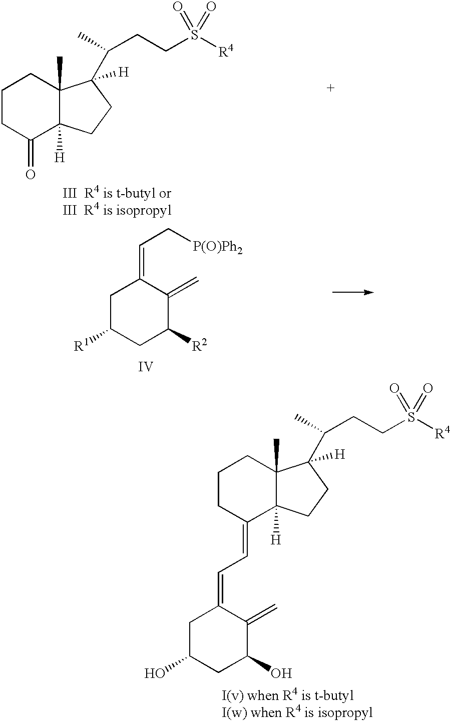

(a) Preparation of a Compound of Formula I(v): A solution of 79 mg (0.13 mmol, 1.0 eq) of phosphine oxide (−)-IV in 1.5 mL of anhydrous THF was cooled to −78° C. and treated with 85 μL (0.15) mmol, 1.0 eq) of 1.7 M solution of phenyllithium in THF. The solution was stirred for 30 min at −78° C. To the solution, was added dropwise a solution of 45 mg (0.13 mmol, 1 eq) of the C,D-ring ketone (+) III (R⁴=t-butyl) in 1 mL of anhydrous THF. After being stirred for 2 hr at the same temperature, the reaction was quenched with 2 mL of a 1:1 mixture of 2N sodium potassium tartrate and 2 NK₂CO₃, extracted with EtOAc (50 mL×2) and washed with brine. The combined organic portions were dried with anhydrous MgSO₄, concentrated in vacuo, and then purified by chromatography (20% Et₂O/hexanes) to afford 30 mg of the coupled product as a colorless oil. The silyl ether was dissolved in 3 mL of anhydrous THF. To the solution, were added 0.17 mL (0.17 mmol, 4 eq) 1 M solution of TBAF in THF, and 23 μL (4 eq of triethylamine). After being stirred for 16 h at rt, the mixture was extracted with EtOAc (50 mL×2) and washed with brine. The combined organic proportions were dried with anhydrous MgSO₄, concentrated in vacuo, and then purified by chromatography (90% EtOAc/hexanes) to afford 20 mg (32%) of enantiomerically rich I(v) as a white solid. The solid was purified by the reverse phase HPLC (C-18 semipreparative column, 50% MeCN/H₂O), 3 ml/min, 262 nm) to afford 11.2 mg of (+)-IIa (1α,3β, ret. time 36 min):

(+)-I(v) (1α,3β): mp. 89-93° C.; [α]²⁵_{D}+63° (c 1.2, EtOH); ¹H NMR (400 Mhz, CDCl₃) δ 6.36 (d, J=11.2 Hz, 1H), 6.01 (d, J=11.2 Hz, 1H), 5.32 (br s, 1H), 4.99 (br s, 1H), 4.41-4.44-4.05 (m, 1H), 4.22 (septet, J=3.2 Hz, 1H), 2.97 (tb, J=12.0, 4.4 Hz, 1H), 2.76-2.85 (m, 2H), 2.56-2.61 (m, 1H), 2.31 (dd, J=13.6, 6.8 Hz, 1H), 1.90-2.06 (m, 7H), 1.47-1.71 (m, 5H), 1.42 (s, 9H), 1.25-1.36 (m, 4H), 0.97 (d, J=6.0, 3H), 0.55 (s, 3H); ¹³C NMR (100 MHz, CDCl₃ δ 147.49, 142.54, 133.12, 124.76, 117.21, 111.86, 70.79, 66.79, 58.98, 56.18, 56.07, 45.91, 45.23, 42.96, 42.78, 40.38, 35.52, 28.98, 27.49, 26.18, 23.55, 23.48, 22.20, 18.54, 12.04; UV (MeOH) λₘₐₓ 264 nm (ε 17,000); MS m/z (70 eV, Cl) 482 (M+NH₄⁺); HRMS m/z (M+) Calcd. 464.2960 for C₂₇H₄₄O₄S, found 464.2971; IR (neat, cm⁻¹) 3391, 2944, 2874, 1275, 1113.

(b) Preparation of a Compound of Formula I(w): The C/D-ring ketone (+)-III (R⁴=isopropyl) in 1 mL of anhydrous THF was reacted with a solution of 56 mg (0.10 mmol, 1.1 eq) of phosphine oxide (−)-IV in 1.0 mL of anhydrous THF followed by desilylation as described for I(v) above to afford 7.4 mg (19%) of enantiomerically rich (+)-I(w) as a white solid. The solid was purified by reverse phase HPLC (C-18 semipreparative column, 45% MeCN/H₂O, 3 ml/min, 262 nm) to afford 11.2 mg of (+)-I(w) (1α,3β, ret. time 28 min):

(+)-I(w) (1α,3β): mp.54-56° C.; [α]²⁵_{D}+59°) (c 0.5, EtOH); ¹H NMR (400 MHz, CDCl₃) δ 6.37 (d, J=11.2 Hz, 1H), 6.01 (d, J=11.2 Hz, 1H), 5.33 (ts, J=1.6 Hz, 1H), 4.99 (br s, 1H), 4.41-4.44 (m, 1H), 4.23 (septet, J=3.2 Hz, 1H), 3.11 (septet, J=6.6 Hz, 1H), 2.97 (tb, J=12.0, 4.4 Hz, 1H), 2.79-2.86 (m, 2H), 2.60 (dd, J=13.6, 3.2 Hz, 1H), 2.31 (dd, J=13.6, 6.8 Hz, 1H), 1.90-2.05 (m, 6H), 1.48-1.72 (m, 6H), 1.39 (d, J=6.8, 3H), 1.24-1.36 (m, 4H), 0.97 (d, J=6.4, 3H), 0.56 (s, 3H); ¹³C NMR (100 MHz, CDCl₃) δ 147.53, 142.52, 133.15, 124.80, 117.25, 111.86, 70.83, 66.84, 56.20, 55.88, 52.60, 46.60, 45.91, 45.26, 42.83, 40.39, 35.39, 28.99, 27.53, 27.12, 23.49, 22.22, 18.57, 15.46, 15.26, 12.05; UV (MeOH) λₘₐₓ 263 nm (ε 16,700); MS m/z (70 eV, Cl) 470 (M+NH₄⁺); HRMS m/z (M⁺) Calcd. 450 for C₂₆H₄₂O₄S, found 450.; IR (neat, cm⁻¹) 3432, 2943, 2862, 1467, 1304, 1121.

### Example 11

### Preparation of Compounds I(x), I(y) ad I(z)

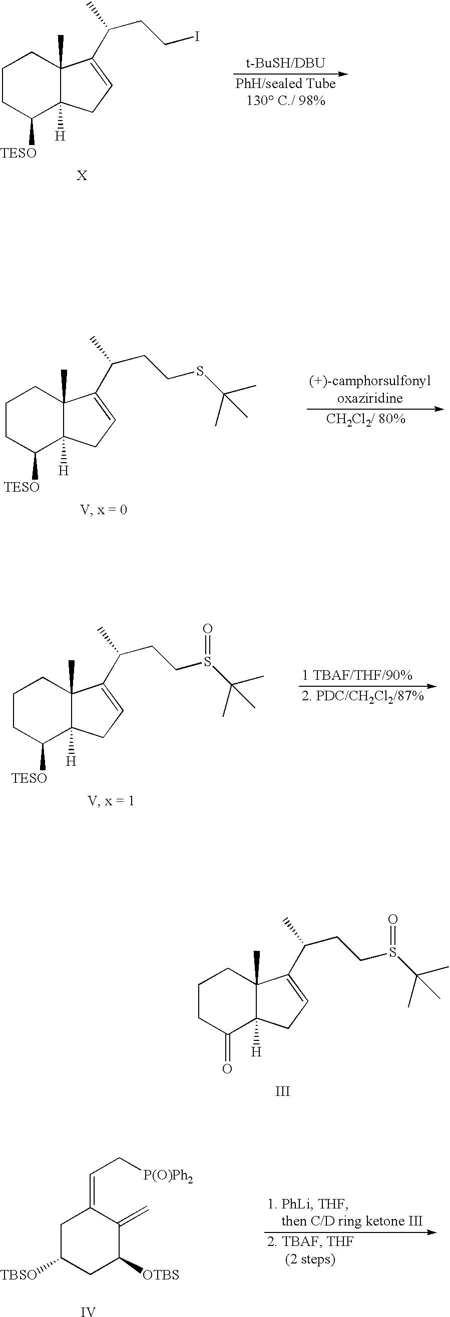

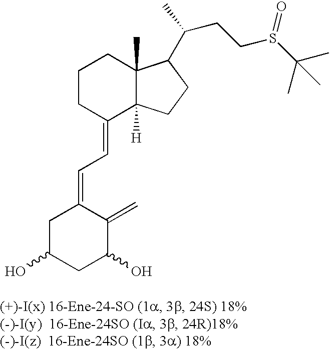

(a) 16-Ene-24-Sulfide (+)-V, x=0. To a solution of the known iodide (Jaekyoo, PhD Thesis, 1997, Johns Hopkins University) X (50 mg, 0.11 mmol) in 1.5 mL of benzene were added 0.025 mL of t-butanethiol (0.19 mmol) and 1,8-diazabicyclo[5.4.0]undec-7-ene (0.025 mL, 0.17 mmol) in hydrolysis tube. The reaction mixture was degased by freeze/thaw cycles (3 times). After 20 h at 130° C., the reaction mixture was cooled to rt, quenched with 3% HCl solution (10 mL) and extracted with ethyl acetate (50 mL×3). The combined organic extract was washed with brine (30 mL), dried over MgSO₄ and concentrated. The crude product was purified by flash column chromatography (6% ethyl acetate/hexanes) to give sulfide (+)-V (x=0) as a colorless oil (44 mg, 98%): [α]²⁵_{D}+18.0 (c 2.0, CHCl₃); ¹H NMR (400 MHz, CDCl₃) δ 5.29 (t, J=1.6 Hz, 1H), 4.12 (d, J=2.4 Hz, 1H), 2.41-2.54 (m, 2H), 2.26 (ddt, J=14.4, 12.0, 1.2 Hz, 1H), 2.13-2.20 (m, 1H), 1.85-1.93 (m, 2H), 1.57-1.81 (m, 5H), 1.26-1.51 (m, 3H), 1.31 (s, 9H), 1.02 (s, 3H), 1.00 (d, J=6.8 Hz, 3H), 0.96 (t, J=8.0 Hz, 9H), 0.57 (q, J=8.0 Hz, 6H); ¹³C NMR (100 MHz, CDCl₃) δ 159.66, 120.00, 68.96, 55.11, 46.70, 41.86, 36.58, 35.75, 34.96, 31.06, 30.78, 26.50, 22.28, 18.76, 18.09, 6.98, 4.65; IR (neat, cm⁻¹) 2956, 2928, 2875, 1457, 1029; HRMS m/z (M+H⁺) calcd 411.3117 for C₂₄H₄₆OSSi, found 411.3109.

(b) 16-Ene-24-Sulfoxdes V (x=1). To a solution of sulfide (+)-V (x=0) (15 mg, 0.036 mmol) in 5.0 mL of CH₂Cl₂ was added (1S)-(+)-camphorsulfonyl oxaziridine (12 mg, 0.052 mmol) at room temperature. The reaction mixture was stirred for 6 h and concentrated. The crude product was purified by flash column chromatography (50% ethyl acetate/hexanes) to give diastereomeric sulfoxides V (x=1) as colorless oil (12 mg, 80%): ¹H NMR (400 MHz, CDCl₃) δ 5.30 (s, 1H), 4.10 (s, 1H), 2.45-1.60 (m, 13H), 1.48-1.33 (m, 3H), 1.21 and 1.20 (two s, 9H), 1.03 and 1.02 (two d, J=6.8 Hz, 3H), 0.97 and 0.96 (two s, 3H), 0.93 and 0.92 (two t, J=8.0 Hz, 9H), 0.54 (q, J=8.0 Hz, 6H); IR (neat, cm⁻¹) 2930, 2875, 1459, 1030.

(c) 16-Ene-8-Keto-24-Sulfoxides III (x=1). To a solution of triethylsilyl-ethers V (x=1) (45 mg, 0.111 mmol) in 5 mL of THF was added tetrabutylammonium fluoride (1 M in THF, 0.13 mL, 0.13 mmol). After 6 h at rt, the reaction mixture was concentrated in reduced pressure. The residue was purified by flashy chromatography (ethyl acetate) to give the corresponding alcohols as coloress oil (31 mg, 90%): ¹H NMR (400 MHz/CDCl₃) δ 5.36 (s, 1H), 4.17 (s, 1H), 2.49-2.16 (m, 4H), 2.04-1.72 (m, 6H), 1.56-1.37 (m, 4H), 1.203 and 1.196 (two s, 9H), 1.05 and 1.04 (two d, J=7.0 Hz, 3H), 1.02 and 1.01 (s, 3H); IR (neat, cm⁻¹) 3404, 2927, 2867, 1455, 1126; HRMS m/z (M⁺) Calcd for C₁₈H₃₂O₂S 313.2201, found 313.2209. To a solution of these alcohols (32 mg, 0.10 mmol) in 7 mL of dry CH₂Cl₂ was added 60 mg of oven dried celite and pyridinium dichlomate (65 mg, 0.17 mmol) at rt. After 4 h, the reaction mixture filtered through flashy silica pad, and then eluted with ethyl acetate. The filtrate was concentrated and purified by flash chromatography (ethyl acetate) to give ketones III as colorless oil (27 mg, 87%): ¹H NMR (400 MHz/CDCl₃) δ 5.33 (s, 1H), 2.85 (m, 1H), 2.47-2.25 (m, 7H), 2.11-1.75 (m, 8H), 1.20 and 1.19 (two s, 9H), 1.11 and 1.10 (two d, J=6.8 Hz, 3H), 0.80 and 0.77 (two s, 3H); IR (neat, cm⁻¹) 2959, 1720, 1458, 1363; HRMS m/z (M⁺) Calcd for C₁₈H₃₀O₂S 311.2045, found 311.2050.

(d) 16-Ene-24-Sulfoxides I(x), I(y) and I(z). To a solution of phosphine oxide (±)-IV (105 mg, 0.18 mmol) in 1 mL of anhydrous THF was treated dropwise with phenyl lithium (1.46 M in cyclohexane-ether, 0.12 mL, 0.18 mmol) at −78° C. The resulting reddish orange solution was stirred at −78° C. for 30 min and then a solution of ketones (+)-IV (x=1) (27 mg, 0.087 mmol) in 1 mL of anhydrous THF was added dropwise. The reaction mixture was stirred until reddish color turned to pale yellow, and then quenched with 3 mL of a 1/1 mixture of 2 N sodium potassium tartrate solution and 2 N K₂CO₃ solution. The aqueous layer was extracted with ethyl acetate (50 mL×3). The combined organic extract was with brine (50 mL), dried over MgSO₄, and concentrated. The residue was purified by preparative TLC (ethyl acetate) to give coupled protected products (38 mg, 64%) and unreacted CD-ring ketones IV (9 mg, 33%). To a solution of the above silyl ethers in 10 mL of THF was tetrabutylammonium floride (1 M in THF, 0.16 mL, 0.16 mmol) and 25 μL of TEA. The solution was stirred at rt for 16 h in dark. The reaction mixture was concentrated in reduced pressure. The residue was purified by preparative TLC (ethyl acetate) to give a mixture of diastereomeric diols I(x), I(y) and I(z) as colorless oil (21 mg, 76%). The diastereomers were separated by reverse phase HPLC (C-18 semi preparative column, 35% MeCN/65% H₂O, 3 mL/min) to give (−)-I(y) as a colorless oil (7 mg, 18% from III, t_{R} 91.5 min), (+)-I(x) as a colorless oil (7 mg, 18% from III, t_{R} 97.2 min) and (−)-I(z) as a colorless oil (7 mg, 18% from III, t_{R}.84.0 min). (−)-I(y): [α]²⁵_{D}−15.4 (c 0.68, CHCl₃); ¹H NMR (400 MHz/CDCl₃) δ 6.37 (d, J=10.8 Hz, 1H), 6.10 (d, J=11.6 Hz, 1H), 5.36 (s, 1H), 5.32 (s, 1H), 5.01 (s, 1H), 4.45 (m, 1H), 4.23 (m, 1H), 2.83 (d, J=12.0 Hz, 1H), 2.60 (d, J=13.6, 1H), 2.46-2.18 (m, 8H), 2.05-1.54 (m, 16H), 1.22 (s, 9H), 1.10 (d, J=6.8 Hz, 3H), 0.67 (s, 3H); ¹³C NMR (100 MHz, CDCl₃) δ 158.02, 147.56, 142.16, 133.20, 124.82, 121.59, 117.00, 111.83, 70.81, 66.82, 58.30, 52.78, 50.04, 45.22, 43.43, 42.84, 35.20, 29.69, 29.41, 28.71, 23.53, 22.92, 22.00, 17.07; IR (neat, cm⁻¹) 3364, 2926, 1640, 1461, 1367, 1012; UV (EtOH) λₘₐₓ 262 nm (ε 17,206); HRMS m/z (M⁺) calcd for C₂₇H₄₂O₃S 447.2933, found 447.2927. (+)-I(x): [α]²⁵_{D}+0.002 (c 0.80, CHCl₃); ¹H NMR (400 MHz/CDCl₃) δ 6.37 (d, J=10.8 Hz, 1H), 6.11 (d, J=11.6 Hz, 1H), 5.36 (s, 1H), 5.34 (s, 1H), 5.01 (s, 1H), 4.44 (m, 1H), 4.24 (m, 1H), 2.83 (d, J=12.4 Hz, 1H), 2.60 (d, J=13.6, 1H), 2.50-2.18 (m, 8H), 2.05-1.69 (m, 16H), 1.23 (s, 9H), 1.10 (d, J=6.8 Hz, 3H), 0.69 (s, 3H); ¹³C NMR (100 MHz, CDCl₃) δ 158.63, 147.58, 142.20, 133.15, 124.85, 121.25, 116.98, 111.75, 70.77, 66.83, 58.35, 50.18, 45.20, 43.75, 42.87, 35.12, 32.50, 30.14, 29.68, 29.42, 28.73, 23.51, 22.89, 21.6, 16.93; IR (neat, cm⁻¹) 3304, 2926, 1640, 1462, 1368, 1057; UV (EtOH) λₘₐₓ 262 nm (ε 12,550); HRMS m/z (M⁺) calcd for C₂₇H₄₂O₃S 447.2933, found 447.2923. I(z) [α]²⁵_{D}−15.9 (c 0.68, CHCl₃); ¹H NMR (400 MHz/CDCl₃) δ 6.38 (d, J=11.6 Hz, 1H), 6.10 (d, J=11.6 Hz, 1H), 5.36 (s, 1H), 5.32 (s, 1H), 5.01 (s, 1H), 4.45 (m, 1H), 4.22 (m, 1H), 2.82 (m, 1H), 2.61 (dd, J=13.4, 3.8, 1H), 2.45-2.18 (m, 8H), 2.08-1.51 (m, 16H), 1.22 (s, 9H), 1.10 (d, J=7.2 Hz, 3H), 0.67 (s, 3H); ¹³C NMR (100 MHz, CDCl₃) δ 158.02, 147.26, 142.22, 133.05, 124.82, 121.59, 116.97, 112.49, 71.28, 66.77, 58.29, 52.77, 50.06, 45.43, 43.46, 42.84, 35.18, 29.69, 29.44, 28.69, 23.51, 22.92, 21.97, 17.09; IR (neat, cm⁻¹) 3304, 2916, 1640, 1462, 1367, 1265, 1012; UV (EtOH) λₘₐₓ 262 nm (ε 12,131); HRMS m/z (M⁺) calcd for C₂₇H₄₂O₃S 447.2933, found 447.2933.

### Example 12

### Preparation of Isopropyl Phenyl Sulfone (VII, R⁴=Ph, R⁶, R⁷=Me)

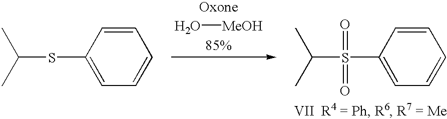

To a solution of isopropyl phenyl sulphide (500 mg, 3.28 mmol) in MeOH (20 mL) was added a solution of potassium peroxymonosulfate (2 KHSO₅.KHSO₄.K₂SO₄, Oxone®) (3.03 g, 9.85 mmol) in water (20 mL) at 0° C. The resulting white suspension was warmed to room temperature and then stirred for 5 h. The mixture was diluted with water (10 mL), extracted with EtOAc (80 mL×2), washed with brine, dried over MgSO₄, concentrated in vacuo, and then purified by column chromatography (25% EtOAc/hexanes) to give 512 mg (85%) of isopropyl phenyl sulfone VII (R⁴=Ph, R⁶, R⁷=Me) as a colorless oil: ¹H NMR (400 MHz, CDCl₃) δ 7.74-7.64 (m, 2H). 7.55-7.51 (m, 1H), 7.46-7.42 (m, 2H), 3.08 (septet, J=6.8 Hz, 1H), 1.50 (d, J=6.8 Hz, 6H); ¹³NMR (100 MHz, CDCl₃) δ 136.54, 133.31, 128.73, 128.55, 55.05, 15.26.

### Example 13

### Preparation of Cyclopropyl Phenyl Sulfone VII (R⁴=Ph, R⁶, R⁷=Cyclopropyl)

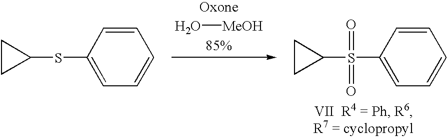

To a solution of cyclopropyl phenyl sulphide (450 mg, 3.00 mmol) in MeOH (15 mL) was added a solution of potassium peroxymonosulfate (2 KHSO₅.KHSO₄.K₂SO₄, Oxone®) (5.52 g, 8.99 mmol) in water (15 mL) at 0° C. The resulting white suspension was warmed to room temperature and then stirred for 5 h. The mixture was diluted with water (10 mL), extracted with EtOAc (60 mL×2), washed with brine, dried over MgSO₄, concentrated in vacuo, and then purified by column chromatography (25% EtOAc/hexanes) to give 494 mg (91%) of cyclopropyl phenyl sulfone VII (R⁴=Ph, R⁶, R⁷=cyclopropyl) as a colorless oil: ¹H NMR (400 MHz, CDCl₃) δ 7.92-7.90 (m, 2H). 7.66-7.62 (m, 1H), 7.58-7.54 (m, 2H), 3.08 (m, 1H), 1.38-1.33 (m, 2H), 1.06-1.00 (m, 2H); ¹³NMR (100 MHz, CDCl₃) δ 140.67, 133.33, 129.19, 127.53, 32.89, 5.94.

### Example 14

### Preparation of 22-Iodo Silyl Ether VI

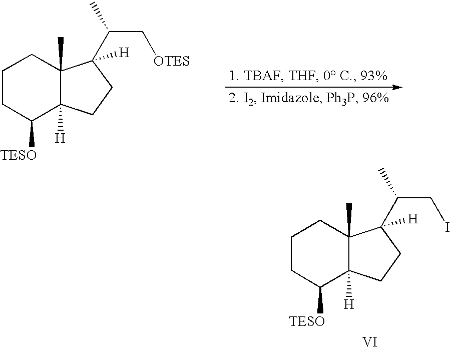

To a solution of bis-silylated diol (508 mg, 1.15 mmol) in 10 mL of anhydrous THF was added 1.15 mL of TBAF (1 M in THF) dropwise at 0° C. After being stirred for 1 h at 0° C., the reaction mixture was extracted with EtOAc (30 mL×2), washed with brine, dried over MgSO₄, concentrated in vacuo, and then purified by column chromatography (20% EtOAc/hexanes) to give 351 mg (93%) of mono-silylated alcohol as a colorless oil.

To a solution of triphenylphosphine (986 mg, 3.76 mmol), imidazole (578 mg, 8.49 mmol) in 20 mL of CH₂Cl₂ was slowly added a solution of iodine (954 mg, 3.76 mmol) in 30 mL of CH₂Cl₂ at 0° C. After 15 min, a solution of mono-silylated alcohol (351 mg, 1.07 mmol) in 10 mL of CH₂Cl₂ was added into the mixture. After being stirred for 6 h at room temperature, the reaction mixture was extracted with EtOAc (100 mL×2), washed with brine, dried over MgSO₄, concentrated in vacuo, and then purified by column chromatography (100% Hexanes) to give 448 mg (96%) of 22-iodo silyl ether VI ( [symbol] =single bond) as a colorless oil: ¹H NMR (400 MHz, CDCl₃) δ 4.03 (m, 1H), 3.33 (dd, J=9.6, 2.8 Hz, 1H), 3.18 (dd, J=9.6, 5.2 Hz, 1H), 1.92-1.75 (m, 3H), 1.70-1.55 (m, 2H), 1.43-1.06 (m, 8H), 0.99 (d, J=6.0 Hz, 3H), 0.94 (t, J=8.0 Hz, 9H), 0.94 (s, 3H), 0.55 (q, J=8.0 Hz, 6H).

### Example 15

### Preparation of Compounds I(aa) and I(bb)

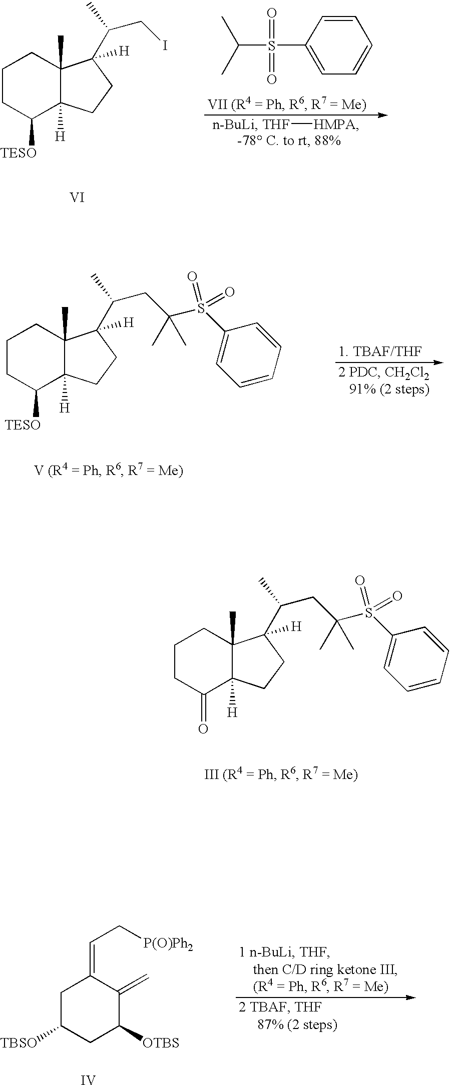

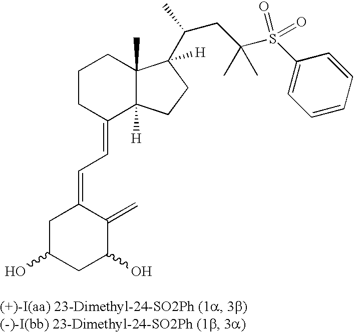

(a) 23-Dimethyl Silyl Ether V (R⁴=Ph, R⁶, R⁷=Me): To a solution of isopropyl phenyl sulfone VII (R⁴=Ph, R⁶, R⁷=Me, Example 12) (38 mg, 0.21 mmol) in THF (3 mL) at −78° C. was added 0.13 mL (0.21 mmol) of n-BuLi (1.6 M in hexanes). After 15 min stirring, 0.3 mL of HMPA was added at −78° C. After another 15 min stirring, a precooled (−78° C.) solution of iodide VI ( [symbol] =single bond, Example 14) (30 mg, 0.069 mmol) in THF (1 mL) was added at −78° C. The reaction mixture was slowly warmed to room temperature and stirred for 2 h, and then quenched with water, extracted with ether (50 mL×2), washed with brine, dried over MgSO₄, concentrated in vacuo, and then purified by column chromatography (20% EtOAc/hexanes) to give 30 mg (88%) of 23-dimethyl silyl ether V (R⁴=Ph, R⁶, R⁷=Me) as a colorless oil: [α]^{24.4}_{D}+27.7 (c 0.57, CHCl₃); ¹H NMR (400 MHz, CDCl₃) δ 7.88-7.85 (m, 2H), 7.66-7.62 (m, 1H), 7.57-7.53 (m, 2H), 4.00 (m, 1H), 1.94-1.84 (m, 2H), 1.81-1.60 (m, 6H), 1.57-1.40 (m, 4H), 0.91(t, J=8.0 Hz, 9H), 0.54 (q, J=8.0 Hz, 6H), 1.34 (s, 3H), 1.30 (s, 3H), 1.26 (s, 3H), 1.14-1.04 (m, 4H), 0.95 (d, J=6.0 Hz, 3H); ¹³NMR (100 MHz, CDCl₃) δ 135.45, 133.39, 130.67, 128.58, 69.32, 64.10, 57.77, 53.08, 42.23, 40.71, 39.40, 34.50, 32.12, 29.70, 27.90, 22.82, 21.10, 17.59, 14.13, 13.28, 6.94, 4.92; IR (neat, cm⁻¹) 2949, 2925, 2872, 1463, 1448, 1378, 1366, 1294, 1282, 1164, 1121, 1075, 1002, 730; HRMS m/z (M⁺+Na⁺) calcd 515.2986 for C₂₈H₄₈O₃SSiNa⁺, found 515.2966.

(b) 23-Dimethyl C,D-ring Ketone III (R⁴=Ph, R⁶, R⁷=Me). To a solution of silyl ether V (R⁴=Ph, R⁶, R⁷=Me) (40 mg, 0.080 mmol) in THF (3 mL) was added 0.24 mL (0.24 mmol) of a 1.0 M solution of TBAF in THF, and then it was stirred at 0° C. for 1 h and stirred overnight at room temperature. The reaction mixture was quenched with water (5 mL), extracted with EtOAc (10 mL×2), washed with brine, dried over MgSO₄, concentrated in vacuo, and then purified by column chromatography (25% EtOAc/hexanes) to give 30 mg (99%) of alcohol as a white solid. To a solution of the C,D-ring alcohol (30 mg, 0.080 mmol) in CH₂Cl₂ (6 mL) was added 80 mg of oven-dried Celite and PDC (84 mg, 0.22 mmol) at room temperature. The reaction mixture was stirred overnight and then passed through a 2 cm pad of flash silica gel and washed with EtOAc. The filtrate was concentrated and purified by column chromatography (33% EtOAc/hexanes) to give 28 mg (91%) of the desired C,D-ring ketone III (R⁴=Ph, R⁶, R⁷=Me) as a white solid: mp 149-151° C.; [α]^{24.7}_{D}+22.3 (c 0.96, CHCl₃); ¹H NMR (400 MHz, CDCl₃) δ 7.85-7.82 (m, 2H), 7.65-7.61 (m, 1H), 7.55-7.51 (m, 2H), 2.41 (dd, J=12.4, 11.2 Hz, 1H), 2.28-2.15 (m, 2H), 2.11-2.06 (m, 1H), 1.96 (m, 1H), 1.91-1.79 (m, 3H), 1.73-1.62 (m, 2H), 1.60-1.43 (m, 6H), 1.32 (s, 3H), 1.27 (s, 3H), 1.00 (d, J=5.6 Hz, 2H), 0.61 (s, 3H); ¹³NMR (100 MHz, CDCl₃) δ 211.67, 135.29, 133.52, 130.63, 128.66, 63.81, 61.92, 57.62, 49.74, 40.87, 39.48, 38.89, 32.31, 28.03, 23.91, 22.34, 21.26, 21.24, 18.90, 12.31; IR (neat, cm⁻¹) 2959, 1715, 1442, 1378, 1305, 1140, 1084, 730, 695; HRMS m/z (M⁺+Na⁺) calcd 399.1964 for C₂₂H₃₂O₃SNa⁺, found 399.1968.

(c) 23-Dimethyl-24-SO₂Ph analogues (+)-I(aa) and (−)-I(bb). A solution of 63 mg (0.11 mmol) of racemic phosphine oxide (±)-IV in 2.0 mL of anhydrous THF was cooled to −78° C. and treated with 67.6 μL (0.11 mmol, 1.6 M in hexanes) of n-BuLi under argon atmosphere. The mixture turned reddish orange and was stirred for 10 min at −78° C. To the solution was added dropwise a solution of 33 mg (0.088 mmol) of the C,D-ring ketone III (R⁴=Ph, R⁶, R⁷=Me) in 1.0 mL of anhydrous THF. The reaction kept going until the reddish orange color faded to yellow (about 4 h). The reaction was quenched by adding 3.0 mL of pH 7 buffer, then warmed to room temperature, extracted with EtOAc (30 mL×2), washed with brine, dried over MgSO₄, concentrated in vacuo, and then purified by column chromatography (10% EtOAc/hexanes) to afford 30 mg (54%) of the coupled product as a colorless oil. The coupled product (30 mg, 0.040 mmol) was dissolved in 3 mL of anhydrous THF, and to this solution was added 0.16 mL (0.16 mmol) of a 1.0 M solution of TBAF in THF. The reaction was run in darkness overnight, then extracted with EtOAc (30 mL×2), washed with brine, dried over MgSO₄, concentrated in vacuo, and then purified by column chromatography (80% EtOAc/hexanes) to give 14 mg (67%) of a mixture of two diastereomers as a white solid. The diastereomers were separated by reverse-phase HPLC (C-18 semipreparative column, 49% MeCN/H₂O, 3.0 mL/min) to afford 2.5 mg (12%) of (+)-I(aa) (1α,3β, t_{R} 116 min) and trace amount of (−)-I(bb) (1β,3α, t_{R} 111 min) as foaming solids. (+)-I(aa): [α]^{24 2}_{D}+25.1 (c 0.12, CHCl₃); ¹H NMR (400 MHz, CDCl₃) δ 7.88-7.85 (m, 2H), 7.67-7.63 (m, 1H), 7.57-7.54(m, 2H), 6.36 (d, J=11.2 Hz, 1H), 6.00 (d, J=11.2 Hz, 1H), 5.32 (s, 1H), 4.98 (s, 1H), 4.43 (m, 1H), 4.23 (m, 1H), 2.82 (m, 1H), 2.60 (m, 1H), 2.31 (m, 1H), 2.03-1.82 (m, 8H), 1.70-1.44 (m, 10H), 1.34 (s, 3H), 1.30 (s, 3H), 1.00 (d, J=5.6 Hz, 3H), 0.54 (s, 3H); ¹³NMR (100 MHz, CDCl₃) δ 147.58, 142.66, 135.38, 133.45, 133.09, 130.66, 128.62, 124.84, 117.21, 111.75, 70.75, 66.81, 63.99, 57.52, 56.31, 45.84, 45.11, 42.79, 40.42, 39.53, 32.85, 28.96, 28.11, 23.46, 22.43, 22.10, 21.30, 21.05, 11.86; IR (neat, cm⁻¹) 3436, 2931, 2861, 1719, 1649, 1443, 1296, 1155, 1126, 1073, 756, 568; UV(MeOH) λₘₐₓ 264 nm (ε 5774); HRMS m/z (M⁺+Na⁺) calcd 535.2853 for C₃₁H₄₄O₄SNa+, found 535.2898.

### Example 16

### Preparation of Compounds I(cc) and I(dd)

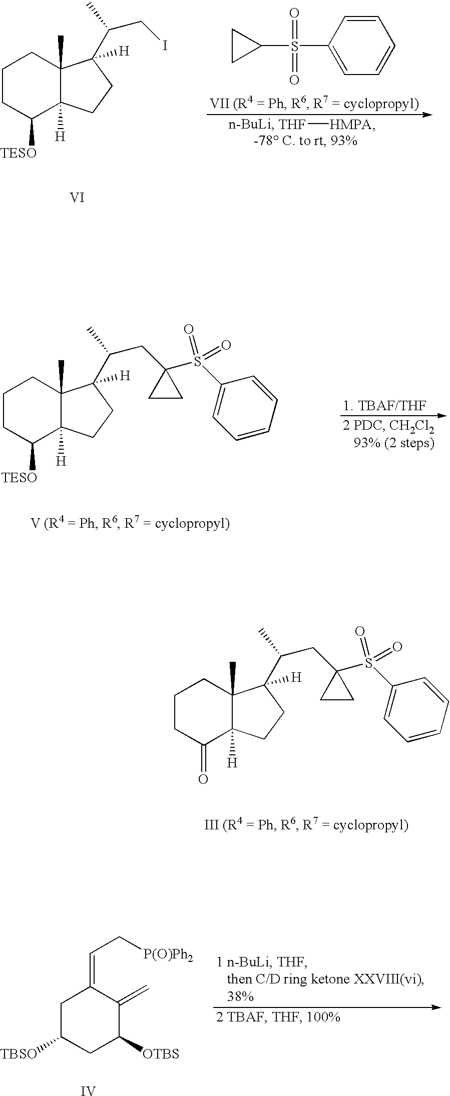

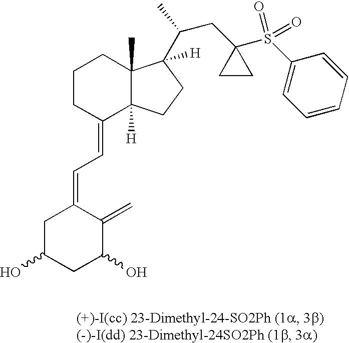

(a) 23-Cyclopropyl Silyl Ether V (R⁴=Ph, R⁶, R⁷=cyclopropyl): To a solution of cyclopropyl phenyl sulfone VII (R⁴=Ph, R⁶, R⁷=cyclopropyl) (Example 13, 50 mg, 0.27 mmol) in THF (3 mL) at −78° C. was added 0.17 mL (0.27 mmol) of nBuLi (1.6 M in hexanes). After 15 min stirring, 0.3 mL of HMPA was added at −78° C. After another 15 min stirring, a precooled (−78° C.) solution of iodide VI (Example 14, 40 mg, 0.091 mmol) in THF (1 mL) was added at −78° C. The reaction mixture was slowly warmed to room temperature and stirred for 3 h, and then quenched with water, extracted with ether (50 mL×2), washed with brine, dried over MgSO₄, concentrated in vacuo, and then purified by column chromatography (15% EtOAc/hexanes) to give 41 mg (93%) of 23-cyclopropyl silyl ether V (R⁴=Ph, R⁶, R⁷=cyclopropyl) as a colorless oil: [α]^{23 8}_{D}+22.2 (c 1.85, CHCl₃); ¹H NMR (400 MHz, CDCl₃) δ 7.90-7.87 (m, 2H), 7.66-7.61 (m, 1H), 7.58-7.53 (m, 2H), 3.98 (m, 1H), 2.10-2.06 (m, 1H), 1.90-1.85 (m, 1H), 1.83-1.71 (m, 2H), 1.66-1.43 (m, 5H), 1.34-1.22 (m, 3H), 1.15-1.01 (m, 2H), 0.94(t, J=8.0 Hz, 9H), 0.96-0.80 (m, 2H), 0.85 (s, 3H), 0.78 (d, J=6.4 Hz, 3H), 0.74-0.69 (m, 3H), 0.54 (q, J=8.0 Hz, 6H); ¹³NMR (100 MHz, CDCl₃) δ 139.18, 133.26, 128.88, 128.73, 69.22, 57.38, 52.93, 42.29, 40.65, 39.29, 37.43, 34.50, 33.56, 27.28, 22.88, 18.81, 17.57, 13.52, 12.57, 12.08, 6.93, 4.90; IR (neat, cm⁻¹) 2949, 2875, 1446, 1304, 1142, 1084, 1021, 974, 807, 727, 690; HRMS m/z (M⁺+Na⁺) calcd 513.2829 for C₂₈H₄₆O₃SSiNa⁺, found 513.2863.

(b) 23-Cycolpropyl C,D-ring Ketone III (R⁴ Ph, R⁶, R⁷ cyclpropyl) To a solution of silyl ether V (R⁴=Ph, R⁶, R⁷=cyclopropyl) (36 mg, 0.073 mmol) in THF (3.0 mL) was added 0.22 mL (0.22 mmol) of a 1.0 M solution of TBAF in THF, and then it was stirred at 0° C. for 1 h and stirred overnight at room temperature. The reaction mixture was quenched with water (4 mL), extracted with EtOAc (10 mL×2), washed with brine, dried over MgSO₄, concentrated in vacuo, and then purified by column chromatography (30% EtOAc/hexanes) to give 27 mg (99%) of alcohol as a colorless oil.

To a solution of the C,D-ring alcohol (27 mg, 0.073 mmol) in CH₂Cl₂ (5 mL) was added 70 mg of oven-dried Celite and PDC (77 mg, 0.21 mmol) at room temperature. The reaction mixture was stirred overnight and then passed through a 2 cm pad of flash silica gel and washed with EtOAc. The filtrate was concentrated and purified by column chromatography (33% EtOAc/hexanes) to give 26 mg (93%) of the desired C,D-ring ketone III (R⁴=Ph, R⁶, R⁷=cyclopropyl) as a white solid: mp 125-127° C.; [α]^{24.5}_{D}+3.62 (c 1.20, CHCl₃); ¹H NMR (400 MHz, CDCl₃) δ 7.88-7.86 (m, 2H), 7.66-7.62 (m, 1H), 7.57-7.53 (m, 2H), 2.35 (dd, J=11.6, 11.2 Hz, 1H), 2.29-2.14 (m, 2H), 2.08-2.04 (m, 2H), 2.00-1.95 (m, 1H), 1.89-1.78 (m, 2H), 1.72-1.41 (m, 6H), 1.23 (m, 1H), 1.00-0.95 (m, 2H), 0.90 (d, J=6.4 Hz, 3H), 0.85 (m, 1H), 0.73-0.69 (m, 1H), 0.59 (s, 3H); ¹³NMR (100 MHz, CDCl₃) δ 211.62, 139.02, 133.43, 128.98, 128.60, 61.74, 57.19, 49.87, 40.85, 39.16, 38.85, 37.85, 33.96, 27.34, 23.90, 18.98, 18.82, 12.76, 12.53, 12.31; IR (neat, cm⁻¹) 2958, 1710, 1446, 1379, 1302, 1141, 1083, 728, 692, 643; HRMS m/z (M⁺+Na⁺) calcd 397.1808 for C₂₂H₃₀O₃SNa⁺, found 397.1807.

(c) 23-Cyclopropyl-24-SO₂Ph analogues (+)-I(cc) and (−)-I(dd). A solution of 57 mg (0.098 mmol) of racemic phosphine oxide (±)-IV in 2.0 mL of anhydrous THF was cooled to −78° C. and treated with 61.1 μL (0.098 mmol, 1.6 M in hexanes) of n-BuLi under argon atmosphere. The mixture turned reddish orange and was stirred for 10 min at −78° C. To the solution was added dropwise a solution of 17 mg (0.046 mmol) of the C,D-ring ketone III (R⁴=Ph, R⁶, R⁷=cyclopropyl) in 1.0 mL of anhydrous THF. The reaction kept going until the reddish orange color faded to yellow (about 2.5 h). The reaction was quenched by adding 2.0 mL of pH 7 buffer, then warmed to room temperature, extracted with EtOAc (20 mL×2), washed with brine, dried over MgSO₄, concentrated in vacuo, and then purified by column chromatography (30% EtOAc/hexanes) to afford 13 mg (38%) of the coupled product as a colorless oil.

The coupled product (13 mg, 0.018 mmol) was dissolved in 3 mL of anhydrous THF, and to this solution was added 0.07 mL (0.07 mmol) of a 1.0 M solution of TBAF in THF. The reaction was run in darkness overnight, then extracted with EtOAc (20 mL×2), washed with brine, dried over MgSO₄, concentrated in vacuo, and then purified by column chromatography (80% EtOAc/hexanes) to give 10 mg (100%) of a mixture of two diastereomers as a white solid. The diastereomers were separated by reverse-phase HPLC (C-18 semipreparative column, 50% MeCN/H₂O, 3.0 mL/min) to afford 2.6 mg (26%) of (+)-I(cc) (1α,3β, t_{R} 74 min) and trace amount of (−)-I(dd) (1β,3α, t_{R} 71 min) as foaming solids. (+)-I(cc): [α]^{24 1}_{D}+18.6 (c 0.22, CHCl₃); ¹H NMR (400 MHz, CDCl₃) δ 7.90-7.87 (m, 2H), 7.67-7.62 (m, 1H), 7.58-7.54(m, 2H), 6.36 (d, J=11.2 Hz, 1H), 5.99 (d, J=11.2 Hz, 1H), 5.33 (s, 1H), 4.99 (s, 1H), 4.43 (m, 1H), 4.23 (m, 1H), 2.79 (m, 1H), 2.59 (m, 1H), 2.30 (m, 1H), 2.10-1.87 (m, 4H), 1.82-1.74 (m, 2H), 1.28-1.19 (m, 2H), 1.11-1.07 (m, 2H), 1.67-1.53 (m, 8H), 1.00-0.93 (m, 2H), 0.86 (d, J=6.4 Hz, 3H), 0.74-0.68 (m, 2H), 0.50 (s, 3H); ¹³NMR (100 MHz, CDCl₃) δ 147.61, 142.71, 139.14, 133.34, 133.06, 128.94, 128.68, 124.88, 117.18, 111.78, 70.83, 57.13, 56.17, 45.96, 45.23, 42.86, 40.36, 39.26, 37.78, 34.45, 28.97, 27.54, 23.45, 22.21, 18.99, 12.64, 12.23, 12.04; IR (neat, cm⁻¹) 3401, 2944, 2861, 1647, 1445, 1303, 1142, 1077, 1053, 721, 691, 573.

### Example 17

### Preparation of Compounds I(ee) and I(ff)

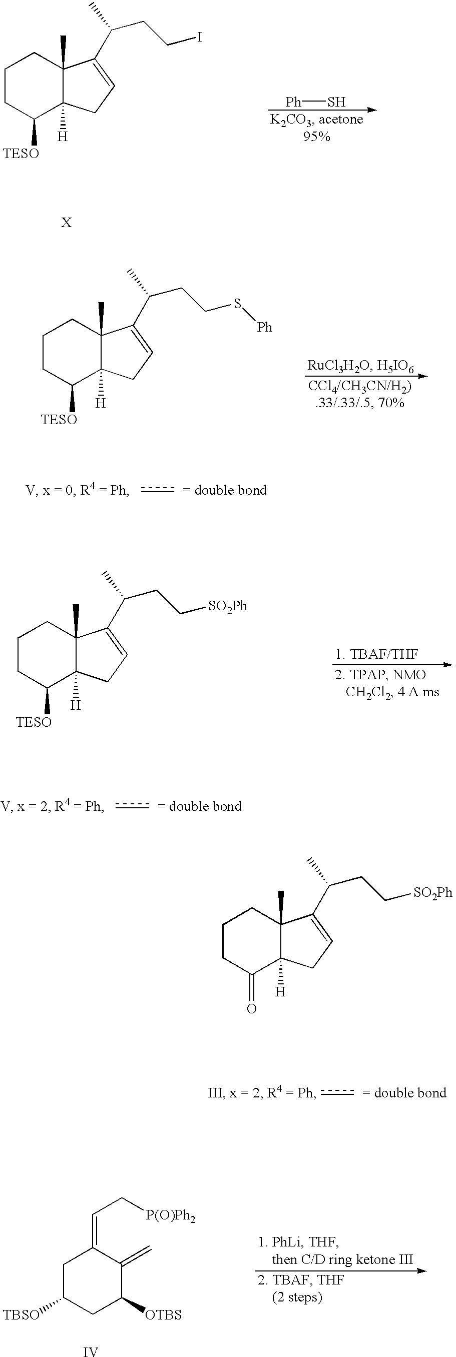

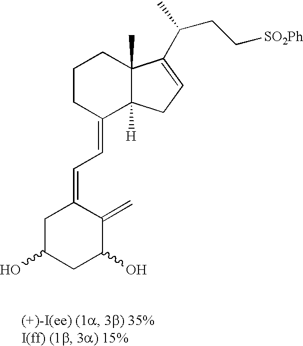

(a) Compound V (x=0, [symbol] =double bond, R⁴=Ph). To a flask, 25 mL, containing iodide X ( [symbol] =double bond) (45 mg, 0.100 mmol) was added acetone (2 mL), K₂CO₃ (70 mg, 0.502 mmol) and finally thiophenol (52 μL, 0.502 mmol) via a syringe. This mixture was stirred at rt. for 1.5 h and quenched with pH 7.0 phosphate buffer (2 mL). The reaction was extracted with Et₂O (3×, 20 mL), dried over MgSO₄, reduced under pressure and purified by silica gel chromatography (100% petroleum ether) to give 45 mg of product as an oil (95%): [α]²⁵_{D}+18.02 (c 0.3925, CHCl₃); ¹H NMR (400 MHz, CDCl₃) δ 7.27 (m, 4H), 7.14 (m, 1H), 5.25 (m, 1H), 4.10 (d, J=2.4 Hz, 1H), 2.91 (ddd, J=12.8, 9.6, 5.6 Hz, 1H), 2.80 (ddd, J=12.8, 9.2, 6.0 Hz, 1H), 2.21 (m, 2H), 1.93-1.77 (m, 3H), 1.72-1.58 (m, 4H), 1.50-1.39 (m, 2H), 1.33 (dt, J=12.8, 3.6 Hz 1H), 0.99 (s, 3H), 0.96 (d, J=6.8 Hz, 3H), 0.94 (t, J=8.0 Hz, 9H), 0.55 (q, J=8.0 Hz, 6H); ¹³C NMR (100 MHz, CDCl₃) δ 159.30, 136.93, 128.84, 128.76, 125.58, 120.22, 68.91, 55.07, 48.77, 46.65, 35.68, 34.91, 31.69, 31.05, 30.74, 22.36, 18.72, 18.04, 6.94, 4.91; IR (CHCl₃, cm⁻¹) 3025, 2954, 1586, 1456, 1028; HRMS m/z (M⁺) calcd 453.261780 for C₂₆H₄₂OSSiNa⁺ found 453.26329.

(b) Compound (+)-V (x=2, [symbol] =double bond, R⁴=Ph. To a flask, 10 mL, was sequentially added sulfide V (x=0, [symbol] =double bond, R⁴=Ph) (40 mg, 0.093 mmol), CCl₄ (0.5 mL), CH₃CN (0.5 mL), H₂O (1 mL) and H₅IO₆ (45 mg, 0.195 mmol). This mixture was stirred vigorously for 5 min at rt., after which was added RuCl₃.H₂O (0.4 mg, 0.0018 mmol) turning the reaction a dark green color. The reaction was stirred until all starting material and intermediate sulfoxide had disappeared by TLC (˜2 h) and then passed over a plug of silica gel. The organics were reduced under pressure and purified by silica gel chromatography (85% petroleum ether, 15% ethyl acetate) to give 30 mg of product as an oil (70%): [α]²⁵_{D}+21.5 (c 0.893, CHCl₃); ¹H NMR (400 MHz, CDCl₃) δ 7.90 (m, 2H), 7.65 (m, 1H), 7.57 (m, 2H), 5.11 (m, 1H), 4.09 (d, J=2.4 Hz, 1H), 3.12 (ddd, J=14.0, 10.8, 4.8 Hz, 1H), 2.97 (ddd, J=14.0, 11.2, 5.6 Hz, 1H), 2.20 (tt, J=12.8, 1.2 Hz, 1H), 2.07 (m, 1H), 1.89-1.72 (m, 4H), 1.69-1.54 (m, 2H), 1.48-1.37 (m, 2H), 1.25 (m, 2H), 0.96 (d, J=6.8 Hz, 3H), 0.94 (t, J=8.0 Hz, 9H), 0.92 (s, 3H), 0.55 (q, J=8.0 Hz, 6H); ¹³C NMR (100 MHz, CDCl₃) δ 157.76, 139.07, 133.54, 129.18, 128.05, 121.13, 68.74, 54.98, 54.69, 46.47, 35.52, 34.75, 30.92, 30.68, 28.66, 22.35, 18.70, 17.93, 6.91, 4.87; IR (CHCl₃, cm⁻¹) 3015, 2933, 1448, 1317, 1149, 1083; HRMS m/z (M⁺) calcd 485.251610 for C₂₆H₄₂O₃SSiNa⁺ found 485.25125.

(c) Compound (+)-III (x=2, [symbol] =double bond, R⁴=Ph). In a flask, 25 mL, was dissolved the sulfone V (x=2, [symbol] =double bond, R⁴=Ph) (28 mg, 0.060 mmol) in THF (1.5 mL). To this was added TBAF (195 μL, 0.195 mmol, 1.0 M in THF) via syringe and the reaction was stirred at rt. for 6 h. The reaction was quenched with water, extracted with Et₂O (3×, 25 mL) and reduced under pressure to give 24 mg of crude product, which was used in the next reaction without further purification. The crude alcohol was dissolved in CH₂Cl₂ (1.5 mL), to which 4 A ms (20 mg), NMO (15 mg, 0.130 mmol) and finally TPAP (1.1 mg, 0.0033 mmol) were added. The reaction was vigorously stirred at rt. for 5 h. The crude reaction mixture was passed over a plug of silica and reduced under pressure. The product was then purified by silica gel chromatography (60% hexanes, 40% ethyl acetate) to give 19.1 mg of product (91%): [α]²⁵_{D}+22.8 (c 0.955, CHCl₃); ¹H NMR (400 MHz, CDCl₃) δ 7.89 (m, 2H), 7.64 (m, 1H), 7.57 (m, 2H), 5.16 (m, 1H), 4.09 (d, J=2.4 Hz, 1H), 3.06 (ddd, J=14.0, 10.4, 5.2 Hz, 1H), 2.98 (ddd, J=14.0, 10.4, 5.6 Hz, 1H), 2.80 (m, 1H), 2.40 (ddt, J=15.6, 10.8, 1.6 Hz, 1H), 2.27-2.19 (m, 3H), 2.10-2.01 (m, 2H), 1.99-1.90 (m, 1H), 1.89-1.78 (m, 3H), 1.69 (m, 1H), 1.04 (d, J=7.2 Hz, 3H), 0.73 (s, 3H); ¹³C NMR (100 MHz, CDCl₃) δ 210.41, 155.62, 139.04, 133.68, 129.28, 127.99, 121.82, 62.94, 54.39, 53.51, 40.37, 34.16, 31.81, 28.41, 2707, 23.87, 21.57, 17.18; IR (CHCl₃, cm⁻¹) 3018, 2935, 1716, 1450, 1337, 1149, 1096; HRMS m/z (M⁺) calcd 369.149483 for C₂₀H₂₆O₃SNa⁺ found 369.14909.

(d) Preparation of Compounds I(ee) and I(ff): Prior to reaction, phosphine oxide (±)-IV and C,D-ring ketone III (x=2, [symbol] =double bond, R⁴=Ph) were azeotrophically dried with benzene and left under vacuum for 24 h. A solution of n-BuLi in hexanes (67 μL, 0.110 mmol) was added dropwise to a cold (−78° C.) solution of phosphine oxide (±)-IV (64 mg, 0.110 mmol) in THF (1.20 mL) under dry argon. The resulting deep red solution was stirred for 40 min, at which time a cold (−78° C.) solution of C,D-ring ketone III (x=2, [symbol] =double bond, R⁴=Ph) (19.1 mg, 0.0551 mmol) in THF (1.0 mL) was added dropwise via cannula. The resulting solution was stirred at −78° C. in the dark for approximately 4 h, after which the dark red color had faded to a light orange color. The reaction mixture was quenched with pH 7.0 phosphate buffer (1 mL), warmed to rt, extracted with Et₂O (3×20 mL), washed with brine, dried over MgSO₄, filtered, concentrated, and purified by silica gel column chromatography (80% hexanes, 20% ethyl acetate) to afford the coupled products as a clear oil (31.5 mg). This oil was immediately dissolved in THF (1.5 mL) and treated with triethylamine (31 mL, 0.221 mmol) and TBAF (221 μL, 0.221 mmol, 1.0 M in THF) and stirred in the dark for 16 h. The reaction mixture was quenched with H₂O (1 mL), extracted with EtOAc (3×15 mL), dried over MgSO₄, filtered, concentrated, and purified by silica gel column chromatography (85% ethyl acetate, 15% hexanes) to afford the diol (21 mg) as a mixture of diastereomers. This diastereomeric mixture was separated by HPLC (CHIRALCEL OJ) giving enantiomerically pure, vitamin-D₃ analogs I(ee) and I(ff) in 35% and 15% yield respectively. I(ce): [α]²⁵_{D}+14.7 (c 0.230, CHCl₃); ¹H NMR (400 MHz, CDCl₃) δ 7.90 (m, 2H), 7.66 (m, 1H), 7.58 (m, 2H), 6.35 (d, J=11.2 Hz, 1H), 6.08 (d, J=11.2 Hz, 1H), 5.34 (m, 1H), 5.18 (m, 1H) 5.00 (m, 1H), 4.44 (m, 1H), 4.24 (m, 1H), 3.08 (ddd, J=14, 10.8, 4.8 Hz, 1H), 2.97 (ddd, J=14.0, 10.8, 4.8 Hz, 1H), 2.79 (m, 1H), 2.59 (dd, J=13.6, 3.2 Hz, 1H), 2.32 (m, 2H), 2.17 (m, 2H), 2.07-2.01 (m, 1H), 1.97 (m, 1H), 1.92-1.86 (m, 1H), 1.74 (m, 2H), 1.67-1.51 (m, 2H), 1.40 (m, 1H), 1.02 (d, J=6.8 Hz, 3H), 0.60 (s, 3H); ¹³C NMR (100 MHz, CDCl₃) δ 157.28, 147.62, 141.86, 139.07, 133.60, 133.31, 129.24, 128.05, 124.73, 121.96, 117.08, 111.66, 70.67, 66.85, 58.26, 54.53, 49.82, 45.15, 42.87, 35.05, 31.95, 29.39, 28.63, 28.50, 23.45, 21.53, 16.82; IR (CHCl₃, cm⁻¹) 3283, 2948, 2874, 1486, 1326, 1163, 1093; HRMS m/z (Mt) calcd 505.238298 for C₂₉H₃₈O₄SNa⁺ found 505.236512.

### Example 18

### Preparation of Compounds I(ga) and I(hh)

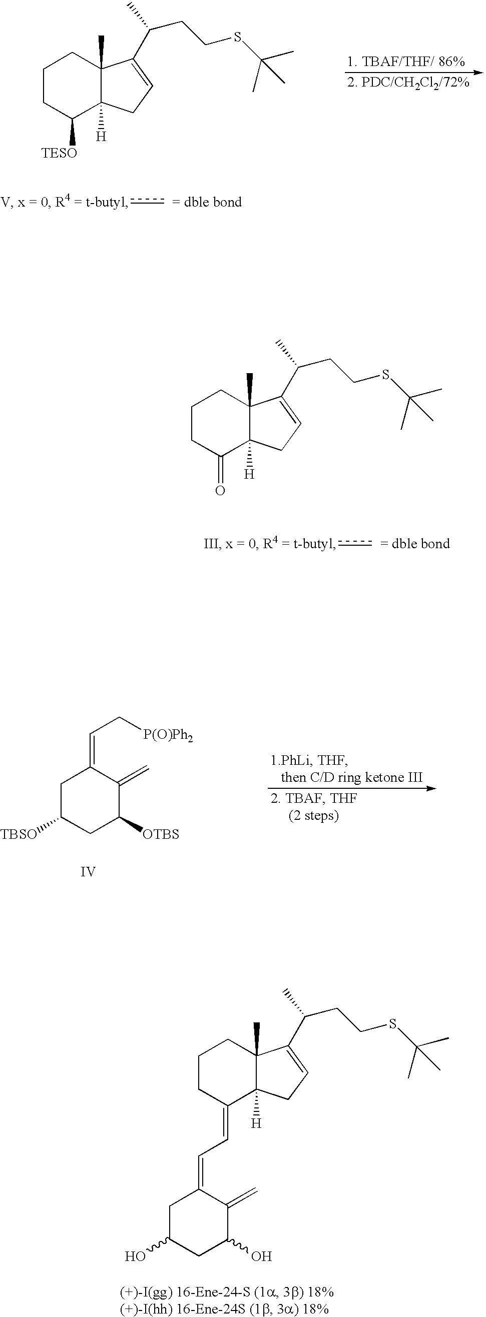

(a) 16-Ene-8-Keto-24-Sulfide (+)-III (x=0, R⁴=t-butyl, [symbol] =dble bond): To a solution of triethylsilyl-ether (+)-V (x=0, R⁴=t-butyl, [symbol] =dble bond, see Example 1 la) (90 mg, 0.22 mmol) in 5 mL of THF was added tetrabutylammonium fluoride (1 M in THF, 0.44 mL, 0.44 mmol). After 5 h at rt, the reaction mixture was concentrated in reduced pressure. The residue was purified by flashy chromatography (20% ethyl acetate/hexanes) to give the corresponding alcohol as a coloress oil (57 mg, 86%): [α]²⁵_{D}+2.6 (c 4.8, CHCl₃); ¹H NMR (400 MHz/CDCl₃) δ 5.31 (s, 1H), 4.16 (s, 1H), 2.52-2.39 (m, 2H), 2.26 (tt, J=13.2, 1.2 Hz, 2H), 2.02-1.70 (m, 6H), 1.65-1.34 (m, 5H), 1.28 (s, 9H), 0.99 (d, J=6.8 Hz, 3H), 1.04 (s, 3H); ¹³C NMR (100 MHz/CDCl₃) δ 159.38, 120.01, 69.06, 54.34, 46.32, 41.80, 36.29, 35.35, 33.87, 31.24, 30.97, 30.20, 26.36, 22.23, 18.32, 17.76; IR (neat, cm⁻¹) 3451, 2926, 1458, 1363; HRMS m/z (M⁺) calcd 296.2174 for C₁₈H₃₂OS, found 296.2178.

To a solution of the alcohol (39 mg, 0.13 mmol) in 7 mL of dry CH₂Cl₂ was added 60 mg of oven dried celite and pyridinium dichlomate (60 mg, 0.16 mmol) at rt. After 16 h, the reaction mixture filtered through flashy silica pad, and then eluted with ethyl acetate. The filtrate was concentrated and purified by flash chromatography (20% ethyl acetate/hexanes) to give ketone (+)-V (x=0, R⁴=t-butyl, [symbol] =dble bond) as a colorless oil (29 mg, 72%): [α]²⁵_{D}+14.8 (c 2.4, CHCl₃; ¹H NMR (400 MHz/CDCl₃) δ 5.29 (s, 1H), 2.83 (dd, J=10.4, 6.4, 1H), 2.52-2.32 (m, 6H), 2.12-1.58 (m, 12H), 1.28 (s 9H), 1.05 (d, J=6.8 Hz, 3H), 0.81 (s, 3H); ¹³C NMR (100 MHz/CDCl₃) δ 210.95, 157.33, 120.53, 63.05, 58.82, 41.86, 40.48, 36.11, 34.28, 32.04, 30.94, 27.04, 26.13, 23.98, 21.59, 17.19; IR (neat, cm⁻¹) 2959, 1720, 1458, 1363; HRMS m/z (M⁺) calcd 294.2017 for C₁₈H₃₀OS, found 294.2018.

(b) 16-Ene-24-Sulfide Calcitriol Analogs I(gg) and I(hh). To a solution of phosphine oxide (±)-IV (50 mg, 0.086 mmol) in 1 mL of anhydrous THF was treated dropwise with phenyl lithium (1.59 M in cyclohexane-ether, 0.054 mL, 0.086 mmol) at −78° C. The resulting reddish orange solution was stirred at −78° C. for 30 min and then a solution of ketone (+)-V (x=0, R⁴=t-butyl, [symbol] =dble bond) (23 mg, 0.080 mmol) in 1 mL of anhydrous THF was added dropwise. The reaction mixture was stirred until reddish color turned to pale yellow, and then quenched with 3 mL of a 1/1 mixture of 2 N sodium potassium tartrate solution and 2 N K₂CO₃ solution. The aqueous layer was extracted with ethyl acetate (50 mL×3). The combined organic extract was with brine (50 mL), dried over MgSO₄, and concentrated. The residue was purified by preparative TLC (ethyl acetate) to give coupled products, unreacted CD-ring ketone (+)-V (x=0, R⁴=t-butyl, [symbol] =dble bond) (9 mg, 39%) and A-ring phosphine oxide IV (21 mg, 41%).

To a solution of the above coupled products in 10 mL of THF was tetrabutylammonium floride (1 M in THF, 0.15 mL, 0.15 mmol). The solution was stirred at rt for 25 h in dark. The reaction mixture was concentrated in reduced pressure. The residue was purified by preparative TLC (ethyl acetate) to give diastereomeric diols I(gg) and I(hh) as colorless oil (16 mg, 47% from (+)-V). The diastereomers were separated by reverse phase HPLC (C-18 semi preparative column, 73% MeCN/27% H₂O, 3 mL/min) to give (−)-I(gg) as a colorless oil (6 mg, 17% from (+)-V, t_{R} 51.5 min) and (−)-I(hh) as a colorless oil (3 mg, 9% from (+)-V, t_{R} 49.4 min). (−)-I(gg):: [α]²⁵_{D}−8.4 (c 0.65, CHCl₃); ¹H NMR (400 MHz/CDCl₃) δ 6.38 (d, J=11.2 Hz, 1H), 6.11 (d, J=11.2 Hz, 1H), 5.34 (s, 2H), 5.02 (s, 1H), 4.45 (m, 1H), 4.24 (m, 1H), 2.83 (d, J=12.4 Hz, 1H), 2.61 (d, J=12.8, 1H), 2.51-2.19 (m, 7H), 2.03-1.49 (m, 16H), 1.30 (s, 9H), 1.05 (d, J=6.8 Hz, 3H), 0.70 (s, 3H); ¹³C NMR (100 MHz, CDCl₃) δ 159.16, 147.58, 142.53, 132.95, 124.92, 120.59, 116.83, 111.72, 70.74, 66.84, 58.35, 50.06, 45.19, 42.83, 41.87, 36.26, 35.21, 32.34, 31.00, 29.68, 29.38, 28.76, 26.29, 23.58, 21.53, 16.86; IR (CHCl₃, cm⁻¹) 3352, 2925, 1458, 1364, 1216, 1055; UV (EtOH) λₘₐₓ 262 nm (ε 17,253); HRMS m/z (M⁺) calcd 430.2906 for C₂₇H₄₂O₂S, found 430.2901. (−)-I(hh): [α]²⁵_{D}−23.0 (c 0.37, CHCl₃); ¹H NMR (400 MHz/CDCl₃) δ 6.39 (d, J=11.2 Hz, 1H), 6.10 (d, J=11.2 Hz, 1H), 5.32 (s, 2H), 5.02 (s, 1H), 4.45 (m, 1H), 4.22 (m, 1H), 2.83 (d, J=12.4 Hz, 1H), 2.63 (dd, J=13.6, 3.6, 11H), 2.52-2.17 (m, 9H), 2.03-1.51 (m, 12H), 1.30 (s, 9H), 1.05 (d, J=7.2 Hz, 3H), 0.70 (s, 3H); ¹³C NMR (100 MHz/CDCl₃) δ 159.17, 147.14, 142.60, 132.79, 124.95, 120.61, 116.83, 112.71, 71.43, 66.77, 58.35, 50.08, 45.49, 42.78, 41.86, 36.29, 35.19, 32.34, 31.01, 29.44, 28.75, 26.29, 23.57, 21.53, 16.88; IR (CHCl₃, cm⁻¹) 3608, 2928, 1459, 1366, 1046; UV (EtOH) λₘₐₓ 263 nm (ε 15,240); HRMS m/z (M⁺) calcd 430.2906 for C₂₇H₄₂O₂S, found 430.2897.

### Example 19

### CYP24 Enzyme Assay (Induced KPK1A-ras Cells)

(i) Material and Reagents:

1,25(OH)₂D₃ 10⁻⁵ M

[3H]-1,25(OH)₂D₃ 25,000 CPM/μL

HPK1A-ras cells

48-well plate

Methanol

Dichlorimethane

Saturated KCl: KCl 30 g, H₂O400 ml

(ii) Procedure:

1. Induction of HPK1A-ras cells (The day before assay)

When the HPK1A-ras cells are 80-90% confluent, add 1 μL 10⁻⁵ M 1,25(OH)₂D₃ to 1 mL medium in the plate (final concentration is 10⁻⁸ M).

2. Preparation of cell suspension

After 18 to 20 hours induction, remove the medium and wash the cell twice with PBS. Then tripsinize the cells from plate, centrifuge (2,000 rpm, 5 min) and suspend cells pellet in DMEM medium+1%BSA.

Count the cells and adjust cells density to 250,000/150 μL, add 150 μL cell suspension to each well in 48-well plate. (including 3 well as no cell control, and 3 well cells without drug or inhibitor as control).

3. Add 25 μL ketoconazole (final concentration 10⁻⁵ M, 10⁻⁶ M, 10⁻⁷ M, 10⁻¹ M) or drugs into each designated well. Keep the plate in 37° C. for 10 min.

4. Preparation of substrate

Take certain amount of DMEM+1%BSA medium (25*Well number+200) μL to a tube, add certain amount of 3H-1,25(OH)₂D₃ (well number+2) μL and certain amount of 100 mM DPPD (well number/5) μL and mix them by vortex.

5. Incubation

Add 25 mL substrate to each well, incubate the plate at 37° C. for 3 hour.

Add 25 mL substrate to counting plate (2 well) as a total count.

6. Lipid extraction and counting

Add 500 μL methanol to each well to stop the reaction, transfer them to tube

Add 250 μL dichloromethane and vortex.

Add 250 μL dichloromethane and 250 μL saturated KCl, and vortex.

Centrifuge at 4000 rpm for 5 min.

Transfer 100 μL of aqueous phase (upper phase) to counting plastic counting plate. Add 600 μL of scintillation fluid to each well. Count the plate in scintillation counter.

7. Calculation enzyme activity

CPM of cell control after subtraction of CPM of NCC is as 100% enzyme activity.

Enzyme activity=(CPM in test compounds well —CPM in NCC well)/(CPM in Cell control−CPM in NCC well)*100%

Dilution of Ketoconazole
Stock 10⁻² M

| Concentration | From | DMEM + | Concentration |
|---|---|---|---|
| (final) | previous step | 1% BSA | (actual) |
| 10⁻⁵ M | 4 | 496 | 8*10⁻⁵ M |
| 10⁻⁶ M | 12.5 | 112.5 | 8*10⁻⁶ M |
| 10⁻⁷ M | 12.5 | 112.5 | 8*10⁻⁷ M |
| 10⁻⁸ M | 12.5 | 112.5 | 8*10⁻⁸ M |

Dilution of test compounds
Stock 10⁻³ M

| | From | DMEM + | |
|---|---|---|---|
| Concentration | previous step | 1% BSA | Concentration |
| (final) | (μL) | (μL) | (actual) |
| 10⁻⁵ M | 10 | 115 | 8*10⁻⁵ M |
| 10⁻⁶ M | 12.5 | 112.5 | 8*10⁻⁶ M |
| 10⁻⁷ M | 12.5 | 112.5 | 8*10⁻⁷ M |
| 10⁻⁸ M | 12.5 | 112.5 | 8*10⁻⁸ M |

(iii) Results:

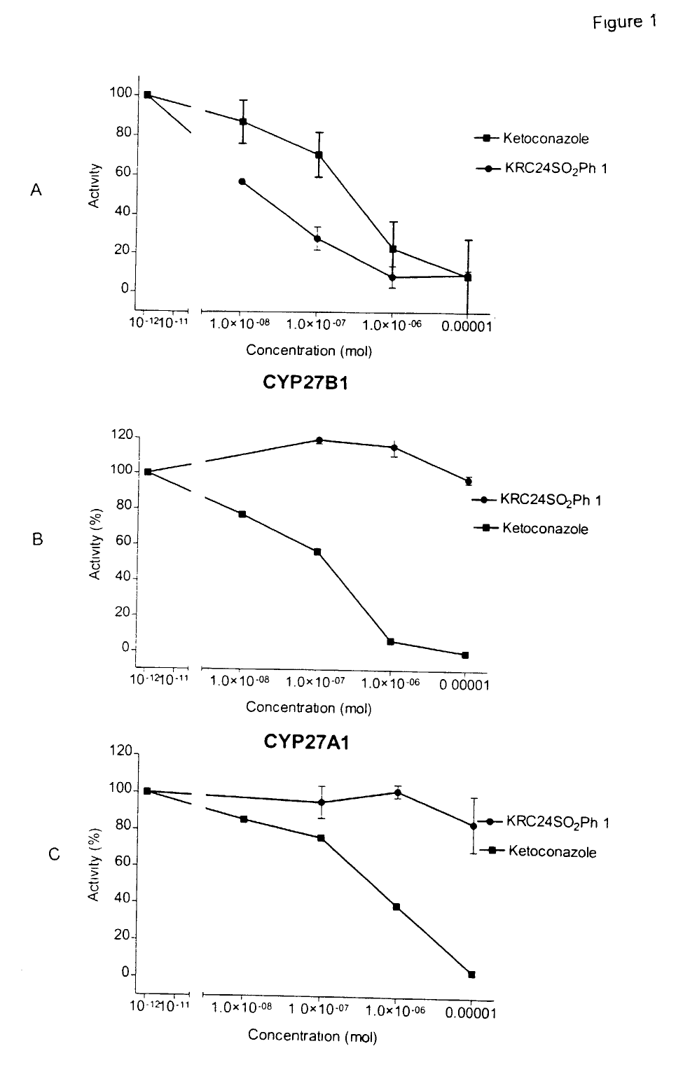

See FIG. 1A for Compound I(a) and see Table 1.

(iv) References:

Ray S, Ray R, Holick M. Metabolism of ³H-I alpha, 25-dihydroxyvitamin D₃ in the cultured human keratinocytes (1995) 59:117-122

Dilworth F J, Scott I, Green A, Strugnell S, Guo Y D, Roberts E A, Kremer R, Calverley, M J, Makin H L J, Jones G. Different mechanisms of hydroxylation site selection by liver and kidney cytochrome P450 species (CYP27 and CYP24) involved in Vitamin D metabolism. (1995) J Biochem 270(28):16766-16774

### Example 20

### Assay of CYP1-Alpha Hydroxylase (Using Transfected COS-1 Cells)

(A) Transit Transfection

(i) Reagent and MATERIAL

1. COS-1 cells (50-80% confluent)

2. FuGene 6 Transfection Reagent

3. PcDNA vector containing CYP-1alpha hydroxylase cDNA (1 μg/μl)

4. DMEM Medium+10% FCS

5. DMEM Medium (serum-free)

6. 6-well plate

(ii) Transfection Cocktail Preparation (The Amount Depends on How Many Wells Transfected)

1. To a sterile tube, add serum-free medium (100 μl per well), Then add FuGene 6 Reagent (3 μl per well). Tap gently to mix. Pay attention to the order. Add FuGene 6 Reagent directly to medium, do not allow undiluted Fugene 6 Reagent to come in contact with plastic surfaces other than the pipette tip.

2. Add DNA solution (1 μg per well) to the prediluted FuGene 6 Reagent from step 2

3. Gently tap the tube to mix the contents. Do not vortex. Incubate for 15 min at room temperature (no more than 45 min).

(iii) Cells Preparation

1. Trypsinize Cos-1 cells, centrifuge cell suspension, suspend cells pellet in DMEM medium+10% FCS.

2. Dilute the cells suspension to 750,000 cell/ml (75 cell/square),

(iv) Transfection

1. Add 1.7 ml DMEM medium+10 O %FCS to each well of 6 well plate.

2. Transfer the correct volume of the cell suspension (200 μl/well) to the transfection cocktail. Mix them gently

3. Add 0.3 ml of the mixture to each well. Make sure that the same amount cells are added to each well. Swirl the wells to ensure even dispersal.

4. Incubate the cells for 24 hours at 37° C., 5% CO₂ until enzyme activity assay.

(B) Enzyme Activity Assay

(i) Reagent and Material

DMEM medium+1% BSA

PBS

[³H-26,27]-25(OH)D₃

DPPD 100 mM

(ii) Procedure

1. Wash cells once with PBS. Be careful, don't disturb the attached cells

2. Add 0.55 ml medium (DMEM+1%BSA) each well.

3. Add 0.025 ml medium containing test compounds

4. Incubate the cells for 10 minutes

5. Add 0.025 ml medium containing [³H-26,27]-25(OH)D₃ (50,000 CPM) and DPPD (0.6 μl stock)

6. Incubate the cells for 2 hour.

7. Add 1.5 ml Methanol to stop reaction

8. Add internal standard.

9. Transfer the medium to labeled tube.

10. Add 0.75 ml dichloromethane, vortex and keep in room temperature for 15 minutes.

11. Add 0.75 ml dichloromethane and 0.75 ml saturated KCl

12. Vortex and centrifuge

13. Remove upper phase and dry the lower phase in Speed-Vac

14. Add 110 μl mobile phase, vortex and centrifuge for 5 min.

15. Transfer 105 μl to the insert in HPLC vial.

16. HPLC analysis conditions:

Solvent: Hexane/isopropanol/methanol (91/7/2)

Column: SIL 3 μm column

Flow rate: 2 ml/min

Detector: UV detector and radioactive detector.

(C) Results

See FIG. 1B for Compound I(a). and see Table 1.

(D) References

Shink T, Shimada H, Wakino S, Anazawa H, Hayashi M, Saruta T, Deluca H, Suda T. Cloning and expression of rat 25-hydroxyvitamin D₃-1-alpha-hydroxylase cDNA. (1997) Pro. Natl Acad Sci 94:12920-12925

Muralidharan K R, Rowland-goldsmith M, Lee S A, Park G, Norman A W, Henry H L, Okamura W H. Inhibitors of 25-hydroxyvitamin D₃-I alpha-hydroxylase: Thiavitamin D analogues and biological evaluation. (1997) J Steroid Biochem. Molec. Biol. 62(1):73-78.

### Example 21

### CYP27A1 Enzyme Assay

(A) Procedure:

As described in:

Dilworth F J, Black S M, Guo Y D, Miller W L, Jones G. Construction of a P450c27 fusion enzyme: a useful tool for analysis of vitamin D₃ 25-hydroxylase (1996) Biochem J 320:267-271

Sawada N, Sakaki T, Ohta M, Inouye K. Metabolism of vitamin D (3) by human CYP27A1 (2000) Biochem Biophys Res Commun 273(3):977-84

(B) Results:

See FIG. 1C for compound I(a) and Table 1.

### Example 22

### VDR Binding Assay

(A) Reagent and Material

1. VDR 9.3 pmol/μl (human, recombinant, Biomol).

2. [³H]-1,25(OH)₂D₃ in ethanol

3. 1,25(OH)₂D₃ in ethanol

4. TEK₃₀₀

Tris-HCl 50 mM

EDTA 1.5 mM

KCl 300 mM

Adjust pH to 7.4 (25° C.),

5. TEDK₃₀₀

TEK₃₀₀

DTT (dithiothreitol) 10 mM (MW 154.24)

6. Tris buffer

22.50 g Tris-HCl

500 ml H₂O

13.25 g Tris-base

500 ml H₂O

Kept in 4° C.

7. Dextran-T70 (Mol 70,000) Pharmacia

8. Charcoal (carbon decolorizing neutral, norit) Fishery

9. Gelatin (G-2625 Sigma)

(B) Reagent Preparation

1. Charcoal dextran solution

(1) Tris buffer

Mix equal amount of Tris-HCl and Tris-base.

(2) Norit decolorizing neutral charcoal 2.0 g

Tris buffer 150 mL

Stirring

(3) Dextran T—70 0.2 g

Tris buffer 50 ml.

(4) Slowly drip the suspended dextran into charcoal solution with stirring.

Keep in refrigerate overnight.

Thirty minute before use, store on ice with continuous mixing

2. TEK₃₀₀/Gelatin solution

50 mg swine gelatin

5 ml TEDK₃₀₀ solution

heating, stirring then cooling to 4° C.

5 ml TEDK₃₀₀ solution

3. Preparation of 1,25(OH)₂D₃ and test compounds in ethanol

1,25(OH)₂D₃: 125, 250, 500, 1000, 2000, 4000 pg/25 μl. (stock 10-5 M/25 μL 100,000 pg/25 μL)

Test compounds: 12,500, 25,000, 50,000, 100,000, 200,000 and 400,000 pg/25 μL. (4*10-5M/25 μL=400,000 pg/25 μL)

| Label | Concentration (ng/mL) | Amount (pg/50 μL) |
|---|---|---|
| | 5.0 | 125 |
| Std F | 10.0 | 250 |
| Std G | 20.0 | 500 |
| Std H | 40.0 | 1000 |
| | 80.0 | 2000 |
| Std I | 160.0 | 4000 |

4. Dilution of VDR:

1 μl stock VDR in 2.5 ml TEDK₃₀₀/Gelatin solution (500 μl/tube), (keep on ice)

(C) Assay:

| | Stan- | NSB | | 1 h | ³H- | 1 h | Reagent C- | On ice | Spin |
|---|---|---|---|---|---|---|---|---|---|
| label | dards | buffer | VDR | RT | 1,25(OH)₂D₃ | RT | charcoal | 30 min | at 4° C. |
| TC | 25 μL | 100 μL | 500 μL | | 50 μL | | 100 μL | | 2000 rpm, |
| (Total) | reagent | reagent L | reagent A | | reagent B | | reagent C | | 10 min |
| NSB | D | 500 μL | 500 μL | | mix all | | mix all | | Add 100 μl |
| (non- | | reagent L | reagent A | | tubes | | tubes | | to counting |
| specific b) | | | mix all | | | | | | rack |
| Max b₀ | | | tubes | | | | | | Count 5-10 |
| binding | | | | | | | | | min |
| Standard | 25 μL | | | | | | | | |
| | of each | | | | | | | | |
| | standard | | | | | | | | |
| Test | 25 μL | | | | | | | | |
| | of each | | | | | | | | |
| | concen- | | | | | | | | |
| | tration | | | | | | | | |
| | of | | | | | | | | |
| | sample | | | | | | | | |

(D) Calculations:

The amount of 1,25(OH)₂D₃ to displace 50 percent [³H]-1,25(OH)₂D₃ from VDR is calculated as B₅₀ for 1,25(OH)₂D₃. The VDR binding of other compounds is calculated as B₅₀ relative to a value of 1 for 1,25(OH)₂D₃.

Dilution of 1,25(OH)D₃

| | Final | | |
|---|---|---|---|
| Concentration | concentration | | |
| (pg/25 ul) | M | 10⁻⁵ M | Ethanol (ul) |
| 4,000 | 2*10⁻⁸ | 6 | 144 |
| 2,000 | 10⁻⁸ | 70 | 70 |
| 1,000 | 5*10⁻⁹ | 70 | 70 |
| 500 | 2.5*10⁻⁹ | 70 | 70 |
| 250 | 1.25*10⁻⁹ | 70 | 70 |
| 125 | 6.25*10⁻¹⁰ | 70 | 70 |

Dilution of test compounds

| Concentration (pg/50 ul) | Final concentration M | 10⁻³ M | Ethanol |
|---|---|---|---|
| 400,000 | 2*10⁻⁶ | 6 | 144 |
| 200,000 | 10⁻⁶ | 70 | 70 |
| 10,000 | 5*10⁻⁷ | 70 | 70 |
| 5,000 | 2.5*10⁻⁷ | 70 | 70 |
| 25,000 | 1.25*10⁻⁷ | 70 | 70 |
| 12,500 | 6.25*10⁻⁸ | 70 | 70 |

(E) Results:

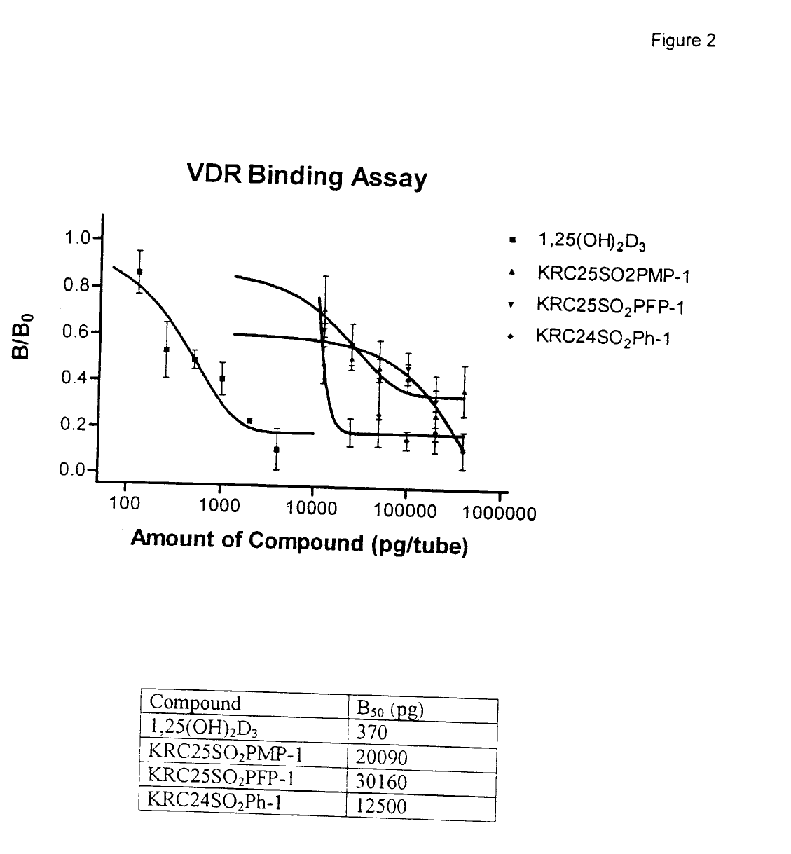

See FIG. 2 and Table 1.

(F) References:

1. Ross T K, Prahl J M, DeLuka H. Overproduction of rat 1,25-dihydroxyvitamin D₃ receptor in insect cells using the baculovirus expression system. (1991) Proc Natl Acd Sci USA 88:6555-6559

2. Wecksler W R, Norman A W. An hydroxylapatite batch assay for the quantitation of 1alpha, 25-dihydroxyvitamin D₃-receptor complexes (1979) Anal Biochem 92:314-323

### Example 23

### Transcriptional Activity Assay

(A) Reagent and Material:

pSG5-hVDR1/3 from DRs. Mark Haussler and Kerr Whitfield, (University of Arizona, Tucson, Ariz.); hVDR1/3 DNA inserted into the EcoRI site of pSG5vector (CT4)⁴TKGH from DRs. Mark Haussler and Kerr Whitfield, (University of Arizona, Tucson, Ariz.); Four copies of the CT4 synthetic rat osteocalcin VDRE ligated and annealed into pTKGH vector which has a thymidine promoter linked to the human GH gene.

hGH ELISA kit. Boehringer Mannheim

Fugene 6 transfection reagent

COS-1 cells

DMEM medium and DMEM medium+10%FCS

1,25(OH)₂D₃ and test compounds

(B) Transfection:

1. Subculture COS cells into 24-well plate (5,000 celll/well) one day before transfection.

2. Cocktail preparation (the amount depends on how may wells transfected).

(1) To a sterile tube, add serum-free medium (100 μl per well), Then add FuGene 6 Reagent (0.6 μl per well). Tap gently to mix. Pay attention to the order. Add FuGene 6 Reagent directly to medium, do not allow undiluted Fugene 6 Reagent to come in contact with plastic surfaces other than the pipette tip.

(2) Add DNA solution (pSG5-hVDR1/3 and (CT4)⁴TKGH vectors) (0.1 μg each per well) to the prediluted FuGene 6 Reagent from step 2

(3) Gently tap the tube to mix the contents. Do not vortex. Incubate for 15 min at room temperature (no more than 45 min).

3. Remove the medium and replaced by 0.4 ml fresh medium

4. Add the 100 μl cocktail to each well in drop-wise manner.

(C) Treatment of Transfected Cells with Different Concentrations of 1,25(OH)₂D₃ and Test Compounds:

30 min to 1 hour after transfection, 1,25(OH)₂D₃ (as control) and test compounds are added to the medium in 20 μl medium. The concentration range for 1,25(OH)₂D₃ is 10⁻¹⁰ to 10⁻⁸ M (10⁻¹⁰, 3*10⁻⁹, 10⁻⁹, 3*10⁻⁸, 10⁻⁸ M) and for test compounds is from 3*10⁻⁹M to 10⁻⁷M (3*10⁻⁹, 10⁻⁹, 3*10⁻⁸, 10⁻⁸, 3*10⁻⁸, 10⁻⁷ M). Incubation continues for 24 hours.

(D) Measurement of GH Content in Medium:

After 24 hour incubation, 200 gL diluted aliquots of medium (dilution of 20-50 times) are used for human GH determination. Sample is assayed according to instruction of hGH ELISA kit.

(E) Results:

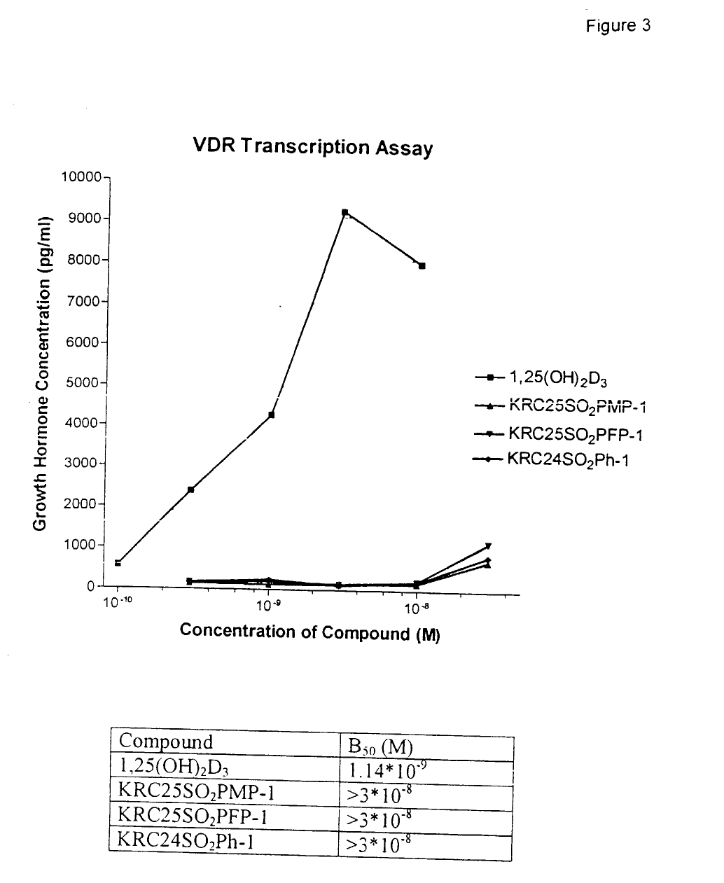

See FIG. 3 and Table 1.

(F) References

Hashimoto Y, Ikeda I, Ikeda M, Takahashi Y, Hosaka M, Uchida H, Kono N, Fukui H, Makino T, Honjo M. Construction of a specific and sensitive sandwich enzyme immunoassay for 20 KD human growth hormone (1998) J Immunol Methods 221:77-85

Jone G, Byford V, Makin H L J, Kremer R, Rice R H, deGraffenried L A, Knutson J C, Bishop C W. Anti-proliferative activity and target cell catabolism of the vitamin D analogue 1alpha, 24(OH)₂D2 in normal and immortalized human epidermal cells (1996) Biochem Pharmacol 52:133-140

### Example 24

### DBP Binding Assay (Human Plasma)

(A) Reagents:

1. Tris buffer:

22.50 g Tris-HCl

500 ml H₂O

2. 13.25 g Tris-base

500 ml H₂O

Kept in 4° C.

3. Dextran-T70 (Mol 70,000) Pharmacia

4. Charcoal (carbon decolorizing neutral, norit) Fishery

5. DBP (vitamin D binding protein) (human plasma)

6. [³H] 25(OH)D₃

7. Gelatin (G-2625 Sigma)

(B) Reagent Preparation:

1. Tris buffer

Mix equal volume of two Tris buffer.

2. Dextran coated charcoal solution

(1) preparation of charcoal solution

Norit decolorizing neutral charcoal 2.0 g

Tris buffer 150 mL

Stirring

(2) preparation of dextran solution

Dextran T—70 0.2 g

Tris buffer 50 ml

(3) preparation of dextran coated charcoal solution

Slowly drip the dextran solution into charcoal solution with stirring.

Keep in refrigerate overnight.

Thirty minute before use, keep it on ice with continuous mixing.

This solution can be kept in 4° C. for 2 month.

3. Tris buffer/Gelatin solution

250 mg swine gelatin

50 ml Tris buffer

heating, stirring and cooling on ice.

Prepared just before use.

4. DBP solution

Human plasma is diluted to 1:5000 with Tris buffer/gelatin solution

5. Dilution of Standard 25(OH)D₃

Stock 10,000 pg/50 μl

Diluted to 0, 62.5, 125, 250, 500, 750, 1000, 10,000 pg/50 μl with ethanol

6. Dilution of Standard 1,25(OH)₂D₃

Stock 200,000 pg/50 μI (10⁻⁵ M/50 ul)

Diluted to 6,250, 12,500, 25,000, 50,000, 100,000, 200,000 pg/50 μl with ethanol

7. Dilution of test compounds

Stock 200,000 pg/50 μl (10⁻³ M)

Diluted to 12,500, 25,000, 50,000, 100,000, 200,000 and 400,000 pg/50 μl with ethanol

8. [³H-26,27]-25(OH)₂D₃ solution

The stock solution is diluted in Tris buffer, 20,000 CPM/50 μl.

(C) Assay

| | | Test | 3H- | | Sup | | Charcoal | | | |
|---|---|---|---|---|---|---|---|---|---|---|
| | 25(OH) | compound | 25(OH) | DBP | erm | Incubation | dextran | On | | |
| Label | D₃ | s (μl) | D₃ (μl) | (μl) | ix | (Rm T) | (μl) | ice | Centrifuge | Counting |
| 1-3 | — | — | 50 | — | 600 | — | — | | — | — |
| (total) | | | | | 600 | | | | | |
| 4-8 | — | — | 50 | 500 | | — | — | | — | — |
| STD 5- | 50 | — | 50 | | — | 4 h | 200 | 1 h | 2000 rpm | 200 μl |
| 35 | | | | | | | | | 15 min, | Super + |
| Test | — | 50 | 50 | | — | | | | 4° C. | 600 μl |
| 36- | | | | | | | | | | Supermix |

(D) Calculation:

The amount of 25(OH)D₃ to displace 50 percent [³H]-25(OH)D₃ is calculated as B₅₀ for 25(OH)D₃ DBP binding. The DBP binding of other compounds is calculated as B₅₀ relative to a value of 1 for 25(OH)D₃.

(E) Dilution of 25(OH)D₃:

| | From previous | |
|---|---|---|
| Amount (mol/50 ul) | steps (μl) | Ethanol (μl) |
| 2.5*10⁻¹¹ (5*10⁻⁷ M) | 5*10⁻⁷ M | |
| 2.5*10⁻¹² | 40 | 360 |
| 1.875*10⁻¹² | 90 | 30 |
| 1.25*10⁻¹² | 130 | 130 |
| 6.25*10⁻¹³ | 130 | 130 |
| 3.125*10⁻¹³ | 130 | 130 |
| 1.5625*10⁻¹³ | 130 | 130 |

(F) Dilution of 1, 25(OH)D₃

| | From previous | |
|---|---|---|
| Amount (mol in 50 μl) | steps (μl) | Ethanol (μl) |
| 5*10⁻¹⁰ (10⁻⁵ M) | | |
| 2.5*10⁻¹⁰ | 130 | 130 |
| 1.25*10⁻¹⁰ | 130 | 130 |
| 6.25*10⁻¹¹ | 130 | 130 |
| 3.215*10⁻¹¹ | 130 | 130 |
| 1.625*10⁻¹¹ | 130 | 130 |

(G) Dilution of Test Compounds:

| | From previous | |
|---|---|---|
| Amount (mol in 50 μl) | steps (μl) | Ethanol (μl) |
| Stock (10⁻³ M) | | |
| 1.0*10⁻⁹ | 5 | 245 |
| 5.0*10⁻¹⁰ | 130 | 130 |
| 2.5*10⁻¹⁰ | 130 | 130 |
| 1.25*10⁻¹⁰ | 130 | 130 |
| 6.25*10⁻¹¹ | 130 | 130 |
| 3.125*10⁻¹¹ | 130 | 130 |

(H) Results:

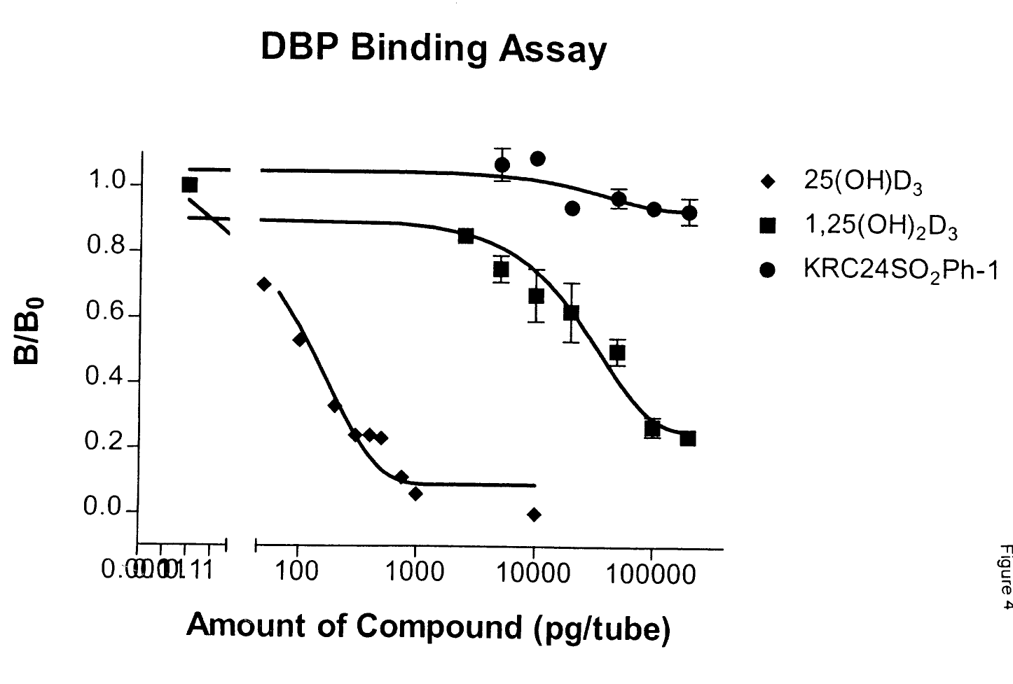

See FIG. 4.

(I) References:

Bouillon R, van Baelen H, Moor P D. Comparative study of the affinity of the serum vitamin D-binding protein. (1980) J Steroid Biochem 13:1029-44.

Jones L, Byrnes B, Palma F, Segev D, Mazur E. Displacement potency of vitamin D₂ analogue in competitive protein-binding assay for 25-hydroxyvitamin D₃, 24,25-dihydroxyvitamin D₃ and 1,25-dihydroxyvitamin D₃ (1980) J Clin Endocrinol Metab 50:773-775

### Example 25

### Calcium Excretion

Compound I(a) was tested for its effect on calcium excretion and weight gain in rats using a protocol described in Posner et al. J. Med. Chem. 41, 3008-3014, 1998. At a concentration that was 20 fold greater than the concentration of calcitriol (1α,25-dihydroxy vitamin D₃), compound I(a) did not show an increase in urinary calcium levels. Compound I(u) was also tested and found to be strongly non-calcemic.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Summary of Biological Activity for Compounds of the Invention

| | | CYP24 | CYP27B1 | CYP27A1 | Cell Anti- |
|---|---|---|---|---|---|
| | | IC₅₀ | IC₅₀ | IC₅₀ | Proliferative |
| Cpd # | Structure | (nM) | (nM) | (nM) | Activity* |
| I(a) | 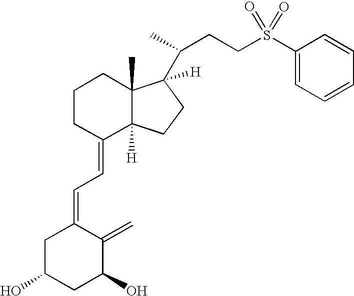 | 28 | >10,000 | >10,000 | |
| I(e) | 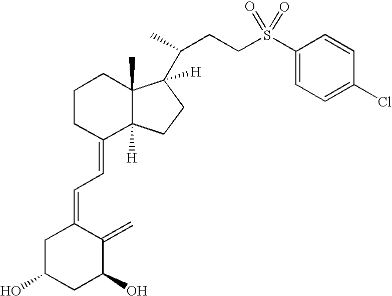 | 94 | >1000 | | |
| I(g) | 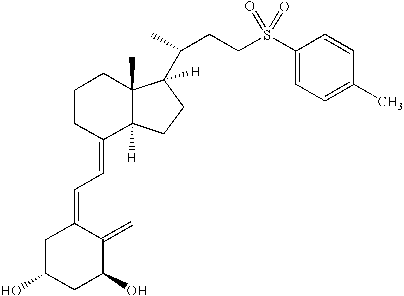 | 212 | >1000 | | |
| I(i) | 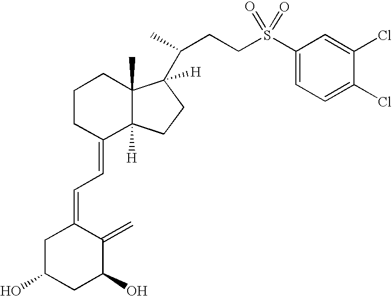 | 92 | >1000 | | |
| I(v) | 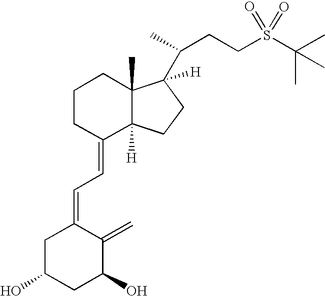 | 219 | >10,000 | | |
| I(w) | 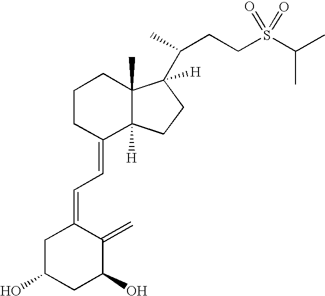 | 90 | 9200 | | |
| I(u) | 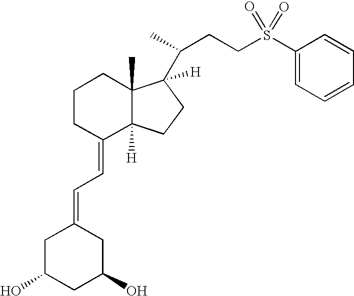 | 160 | | | |
| (−)-I(y) | 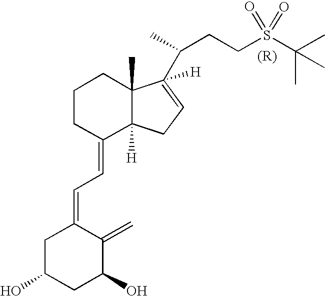 | 146 | >10,000 | | Strong |
| I(cc) | 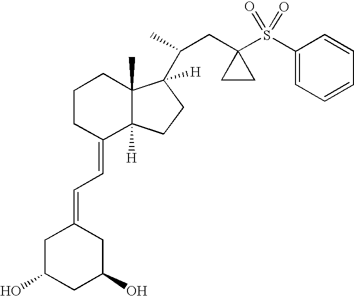 | 27 | | | |
| I(gg) | 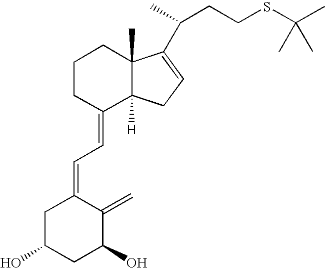 | 467 | 8460 | | |

*compared to calcitrol

We claim:

1. A compound of Formula I, and pharmaceutically acceptable salts, hydrates, solvates and prodrugs thereof: 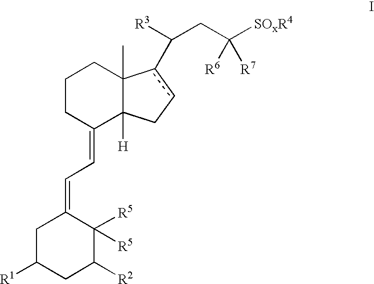
wherein
R¹ and R² are independently selected from the group consisting of OH, OC₁₋₄alkyl, and halo;
R³ is C₁₋₄alkyl;
R⁴ is selected from the group consisting of C₁₋₆alkyl, aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, CH═N—OC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;
R⁵ are either both H or together form ═CH₂;
R⁶ and R⁷ are independently H, C₁₋₄alkyl or are taken together to form a C₃₋₆cyloalkyl ring;
x is 0-2; and [symbol]
represents a single or a double bond.

2. The compound according to claim 1, wherein R¹ and R² are independently selected from the group consisting OH, OCH₃, and fluoro.

3. The compound according to claim 2, wherein R¹ and R² are both OH.

4. The compound according to claim 1, wherein R³ is CH₃.

5. The compound according to claim 1, wherein R⁴ is selected from the group consisting of C₁₋₆alkyl, unsubstituted and substituted phenyl, pyridyl, thienyl, furanyl and pyrrolo.

6. The compound according to claim 5, wherein R⁴ is selected from C₁₋₄alkyl, unsubstituted or substituted phenyl.

7. The compound according to claim 1, wherein both aryl and heteroaryl are either unsubstituted or substituted with 1-3 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, CH═N—OC₁₋₄alkyl, C(O)NHC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂.

8. The compound according to claim 7, wherein both aryl and heteroaryl are either unsubstituted or substituted with 1-2 groups independently selected from methyl, 3-hydroxy-3-pentyl, methoxy, OH, CF₃, OCF₃, halo, NH₂, NMe₂ and CH═N-OMe.

9. The compound according to claim 8, wherein both aryl and heteroaryl are either unsubstituted or substituted with 1-2 groups independently selected from methyl, 3-hydroxy-3-pentyl, Cl, F and CH═N—OMe.

10. The compound according to claim 6, wherein R⁴ is selected from the group consisting of methyl, ethyl, n-propyl, t-butyl, isopropyl, isobutyl, phenyl, 4-chlorophenyl, 3,4-dichloropheny, 4-fluorophenyl, 4-methylphenyl, 3,4-difluorophenyl, 4-(3-hydroxy-3-pentyl)phenyl, 4-(CH═N—OMe)phenyl, 4-methoxyphenyl, 4-trifluormethylpheny and 4-ntirophenyl.

11. The compound according to claim 10, wherein R⁴ is selected from the group consisting of t-butyl, isopropyl, phenyl, 4-chlorophenyl, 3,4-dichloropheny, 4-(3-hydroxy-3-pentyl)phenyl, 4-fluorophenyl and 4-methylphenyl.

12. The compound according to claim 1, wherein R⁶ and R⁷ are independently H, methyl or are taken together to form a C₃₋₄cyloalkyl ring.

13. The compound according to claim 12, wherein R⁶ and R⁷ are both H or are taken together to form a C₃₋₄cyloalkyl ring.

14. The compound according to claim 1, wherein x is 2.

15. The compound according to claim 1, wherein if [symbol] represents a double bond, R⁴ is C₁₋₆alkyl.

16. A compound of Formula I, and pharmaceutically acceptable salts, hydrates, solvates and prodrugs thereof: 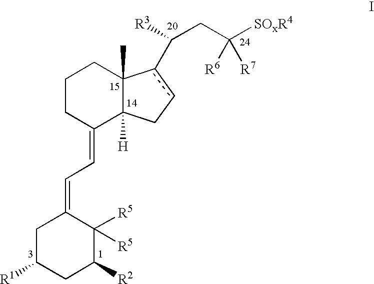
wherein
R¹ and R² are independently selected from the group consisting of OH, OC₁₋₄alkyl, and halo;
R³ is C₁₋₄alkyl;
R⁴ is selected from the group consisting of aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl; OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;
R⁵ are either both H or together form ═CH₂;
R⁶ and R⁷ are independently H. C₄alkyl or are taken together to form a C₃₋₆cyloalkyl ring;
x is 0-2; and [symbol]
represents a single or a double bond.

17. A compound of Formula I, and pharmaceutically acceptable salts, hydrates, solvates and prodrugs thereof: 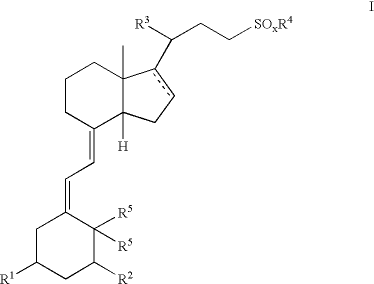
wherein
R¹ and R² are independently selected from the group consisting of OH, OC₁₋₄alkyl, and halo;
R³ is C₁₋₄alkyl;
R⁴ is selected from the group consisting of C₁₋₆alkyl, aryl and heteroaryl with both aryl and heteroaryl being unsubstituted or substituted with 1-5 groups independently selected from C₁₋₄alkyl, hydroxy-substituted C₁₋₆alkyl, OC₁₋₄alkyl, OH, CF₃, OCF₃, halo, SH, SC₁₋₄alkyl, NH₂, NHC₁₋₄alkyl, N(C₁₋₄alkyl)(C₁₋₄alkyl), CN, C(O)OH, C(O)OC₁₋₄alkyl, C(O)NHC₁₋₄alkyl, CH═N—OC₁₋₄alkyl, NHC(O)C₁₋₄alkyl, OC(O)C₁₋₄alkyl, SOC₁₋₄alkyl, SO₂C₁₋₄alkyl, SO₂NHC₁₋₄alkyl and SO₂NH₂;
R⁵ are either both H or together form ═CH₂;
x is 0-2; and [symbol]
represents a single or a double bond.

18. The compound according to claim 1 that is selected from: 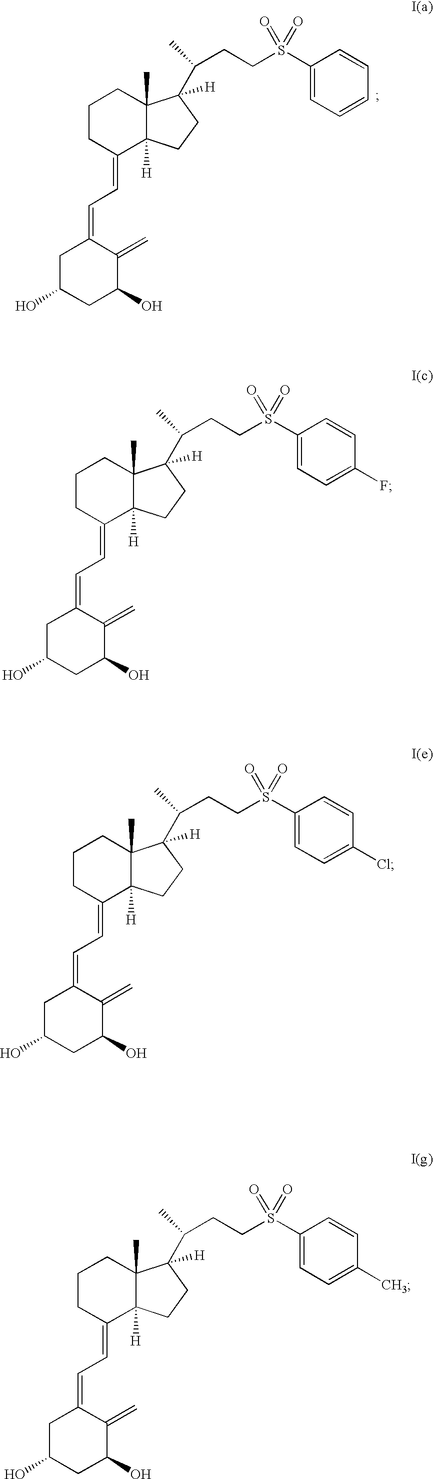 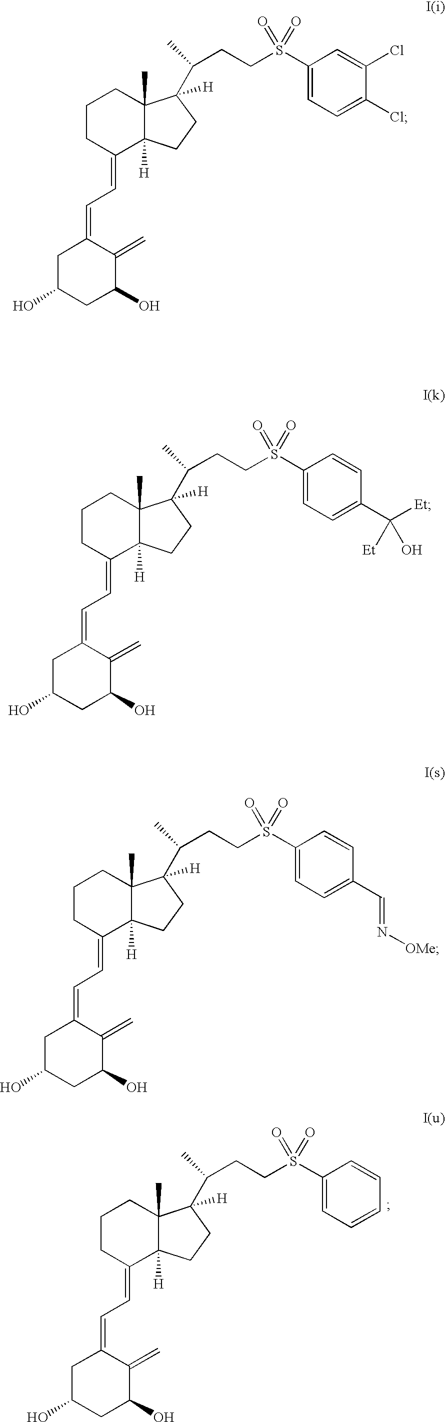 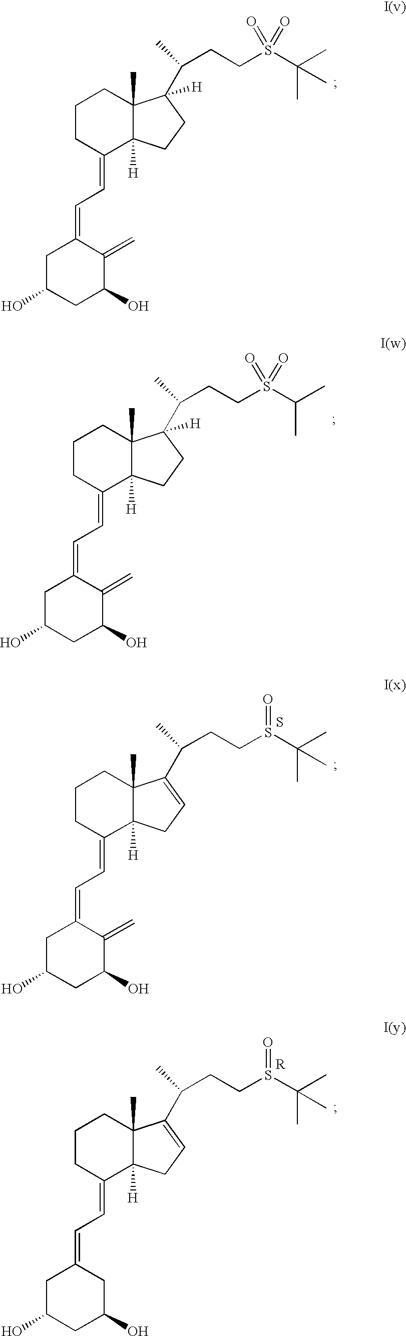 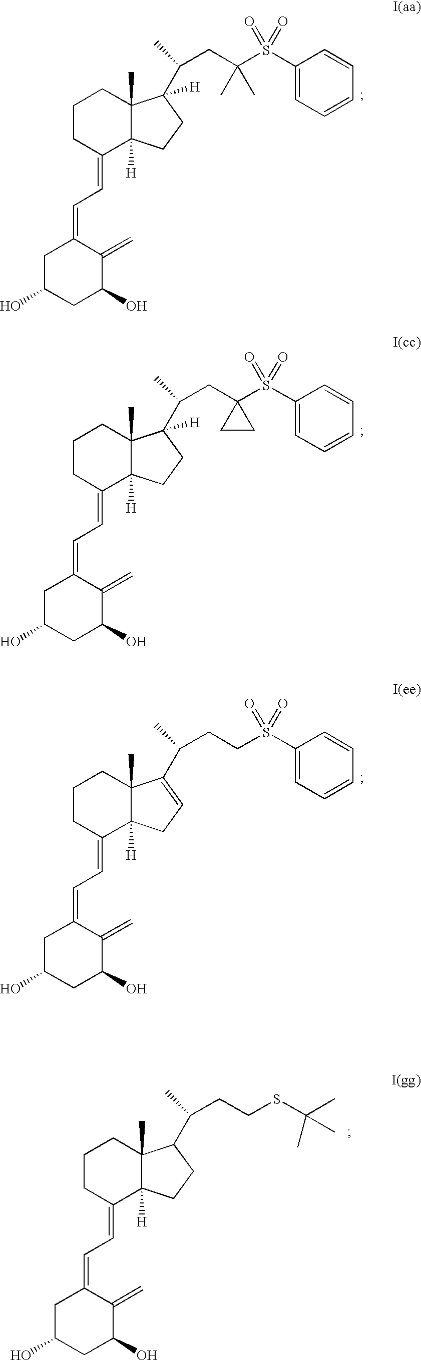
and pharmaceutically acceptable salts, hydrates, solvates and prodrugs thereof.

19. A pharmaceutical composition comprising a compound according to claim 1 and a pharmaceutically acceptable carrier.

20. A method for treating diseases which benefit from a modulation of the levels of 1α,25-dihydroxy vitamin D₃ comprising administering an effective amount of a compound according to claim 1 to a cell or animal in need thereof.

21. A method for treating diseases which benefit from an inhibition of the catabolism of 1α,25-dihydroxy vitamin D₃ comprising administering an effective amount of a compound according to claim 1 to a cell or animal in need thereof.

22. The method according to claim 21, wherein the disease is selected from the group consisting of cancer, dermatological disorders, thyroid disorders, wound healing and bone disorders.

23. The method according to claim 22, wherein the disease is selected from the group consisting of cancer, psoriasis, thyroid disorders and osteoporosis.

24. A method for inhibiting cell proliferation comprising administering an effective amount of a compound according to claim 1 to a cell or animal in need thereof.

25. The method according to claim 24, wherein the cell is a cancer cell.

26. The method according to claim 25, wherein the cancer is selected from breast cancer, lung cancer and prostate cancer.

27. A method of inhibiting CYP24 activity in a cell by administering an effective amount of a compound according to claim 1 to a cell in need thereof.

28. A use of a compound according to claim 1 to modulate the levels of 1α,25-dihydroxy vitamin D₃.

29. A use of a compound according to claim 1 to inhibit the catabolism of 1α,25-dihydroxy vitamin D₃.

30. A use of a compound according to claim 1 to prepare a medicament to modulate the levels of 1α,25-dihydroxy vitamin D₃,

31. A use of a compound according to claim 1 to prepare a medicament to inhibit the catabolism of 1α,25-dihydroxy vitamin D₃.

32. A use of a compound according to claim 1 to inhibit cell proliferation.

33. A use of a compound according to claim 1 to prepare a medicament to inhibit cell proliferation.

34. A use of a compound according to claim 1 to inhibit CYP24 activity.

35. A use of a compound according to claim 1 to prepare a medicament to inhibit CYP24 activity.

36. A method for increasing the efficacy of a vitamin D receptor agonist comprising co-administering an effective amount of a compound according to claim 1 and an effective amount of the vitamin D receptor agonist.

37. The method according to claim 36, wherein the vitamin D receptor agonist is 1α,25-dihydroxy vitamin D₃ (calcitriol).

38. A use of a compound according to claim 1 to increase the efficacy of a vitamin D receptor agonist.

39. A use of a compound according to claim 1 to prepare a medicament to increase the efficacy of a vitamin D receptor agonist.

40. The use according to claim 38, wherein the vitamin D receptor agonist is 1α,25-dihydroxy vitamin D₃ (calcitriol).

41. The use according to claim 39, wherein the vitamin D receptor agonist is 1α,25-dihydroxy vitamin D₃ (calcitriol).
